(19) United States
(12) Patent Application Publication
(10) Pub. No.: US 2023/0257374 A1
(43) Pub. Date: Aug. 17, 2023

(54) NOVEL KRAS G12C PROTEIN INHIBITOR, PREPARATION METHOD THEREFOR, AND USE THEREOF

(71) Applicant: INNOVENT BIOLOGICS (SUZHOU) CO., LTD., Jiangsu (CN)

(72) Inventors: Long ZHANG, Jiangsu (CN); Guowei SONG, Jiangsu (CN); Zhiliang YANG, Jiangsu (CN)

(73) Assignee: INNOVENT BIOLOGICS (SUZHOU) CO., LTD., Jiangsu (CN)

(21) Appl. No.: 17/767,629

(22) PCT Filed: Oct. 9, 2020
(86) PCT No.: PCT/CN2020/120028

§ 371 Date: Apr. 8, 2022

(30) Foreign Application Priority Data
Oct. 10, 2019 (CN) 201910959491.5
Nov. 15, 2019 (CN) 201911120362.3

(51) Int. Cl. C07D 471/04; A61P 35/00

(52) U.S. Cl. CPC C07D 471/04; A61P 35/00

(57) ABSTRACT

The present invention belongs to the field of medicinal chemistry, and relates to a novel KRAS G12C protein inhibitor, a preparation method and use thereof. In particular, the present invention provides a compound of formula I, which can be used as a high-efficiency KRAS G12C protein inhibitor and has various pharmacological activities against tumors, proliferative diseases, inflammation, autoimmune diseases, etc.

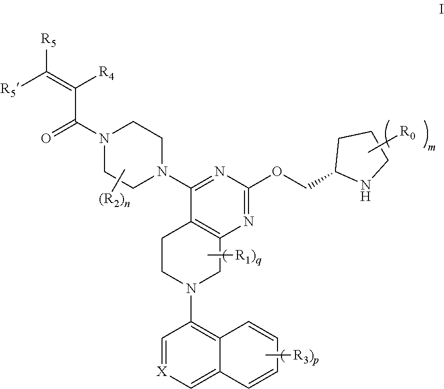

### CROSS-REFERENCE TO RELATED APPLICATIONS

The present invention claims priority to Chinese Patent Application No. 201910959491.5 filed on Oct. 10, 2019 and entitled “NOVEL KRAS G12C PROTEIN INHIBITOR AND PREPARATION METHOD AND USE THEREOF” and Chinese Patent Application No. 201911120362.3 filed on Nov. 15, 2019 and entitled “NOVEL KRAS G12C PROTEIN INHIBITOR AND PREPARATION METHOD AND USE THEREOF”, the contents of which are incorporated herein by reference in their entirety.

### TECHNICAL FIELD

The present invention belongs to the field of medicinal chemistry, and relates to a novel KRAS G12C protein inhibitor, a preparation method thereof, a pharmaceutical composition comprising the same, and pharmaceutical use thereof, particularly use in preparing a medicament for preventing and/or treating a disease at least partially mediated by KRAS G12C protein and/or a contrast agent and/or a tracer for diagnosing the disease.

### BACKGROUND

RAS represents a group of monomeric globular proteins of 189 amino acids (21 kDa molecular weight) that are closely related to each other. It is associated with the plasma membrane, and bind to GDP or GTP. RAS functions as a molecular switch. When RAS comprises bound GDP, it is in a static or off position and is “inactive”. In response to cells exposed to certain growth-promoting stimuli, RAS is induced to exchange its bound GDP for GTP. Where GTP has been bound, RAS is turned “on” and enabled to interact with other proteins (its “downstream targets”) and to be activated. The RAS protein itself has very limited intrinsic capacity to hydrolyze GTP back to GDP and thus to turn itself into the off state. Turning RAS off requires an exogenous protein called GTPase-activating protein (GAP), which interacts with RAS and greatly accelerates the conversion of GTP to GDP. Any mutation that affects the interaction of RAS with GAP or the conversion of GTP back to GDP by RAS will result in long-term activation of proteins and thus long-term signals conducted to a cell that command the cell to constantly grow and divide. Hyperactive RAS signaling may eventually lead to cancer due to the cell growth and division caused by these signals.

Studies on RAS protein inhibitors have been very challenging mainly in that: the affinity of RAS for GDP and GTP is high and can reach a picomolar level, and intracellular GTP concentration is high, such that competitive inhibitors have difficulty in impairing the binding of RAS proteins to GTP; besides, RAS proteins have smooth surfaces and lack effective small molecule binding sites. RAS proteins have been considered as “non-druggable” targets for many years. The ongoing advent of new technologies facilitate the advent of new therapeutic approaches to RAS targets. At present, the research on RAS target signaling pathway inhibitors mainly focuses on the following aspects: direct action on RAS proteins, prevention of RAS binding to GTP, action on upstream and downstream signals, inhibition of RAS interaction with effector proteins, reduction of RAS localization, inhibition of GTPase activity, synthetic lethality, etc.

HRAS, KRAS and NRAS are the most well-known members in the RAS subfamily primarily for their association with many types of cancer. Mutation of any of the three major isoforms of RAS genes (HRAS, NRAS or KRAS) is the most common event in human tumorigenesis. It has been found that approximately 30% of human tumors carry some mutations in RAS genes. Strikingly, KRAS mutations are detected in 25-30% of tumors. In contrast, the rates of oncogenic mutations occurring in NRAS and HRAS family members are much lower (8% and 3%, respectively). In addition, mutations in KRAS are most common in colorectal (45%), lung (35%) and pancreatic (95%) cancers. The most common KRAS mutations occur at residues G12 and G13, as well as residue Q61, in the P-loop. Studies have shown that mutations in RAS genes are associated with many cancers, and that 99% of mutations occur at glycines of positions 12 and 13 as well as glutamate of position 61 (see Y. Pylayova-Gupta, et al., RAS oncogenes: weaving a tumorigenic web [J], Nature reviews cancer, 2011, 11:761-774).

A G12C protein is a protein produced after G12C mutation in KRAS genes (KRAS G12C for short), specifically, mutation of glycine (G) at position 12 into cysteine (C). KRAS G12C is the most frequent form of mutation in KRAS genes, and has been found in about 13% of cancer cases, specifically in about 43% of lung cancer cases and almost 100% of MYH-associated polyposis (familial colon cancer syndrome) cases. In recent years, a series of inhibitors have been developed for the G12C protein. For example, Nature reports an inhibitor with electrophilic groups (e.g., vinylsulfonyl and acryloyl groups), and the co-crystal results suggest an allosteric binding pocket that has never been previously found, which can lead to a change in the structure of Switch I and Switch II in RAS and can impair the binding of KRAS (G12C) protein to GTP (see J. M. Ostrem, et al., K-Ras (G12C) inhibitors allosterically control GTP affinity and effector interactions [J], Nature, 2013, 503:548-551). Cell reports a class of compounds with better inhibitory effects on the G12C protein, which also achieved better results in in vivo experiments in mice (see M. R. Janes, et al., Targeting KRAS Mutant Cancers with a Covalent G12C-Specific Inhibitor [J], Cell, 2018, 172(3):578-589). Some candidate compounds such as MRTX849 from Mirati and AMG-510 from Amgen have also been in clinical studies and have achieved preliminary clinical therapeutic effects.

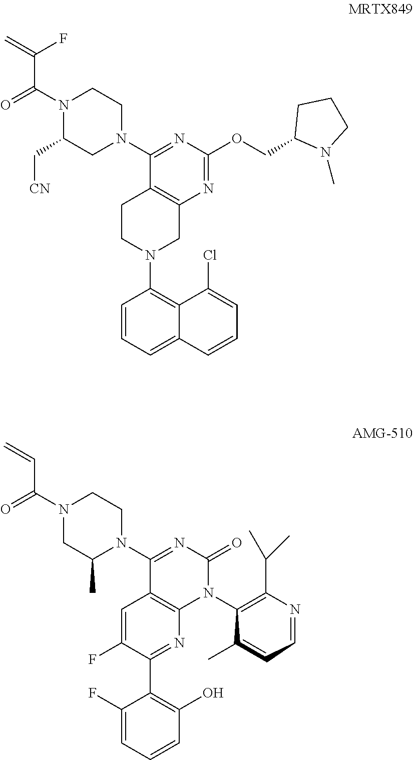

Although some candidate compounds for the KRAS G12C target have entered the clinical research stage, the early candidate compounds generally have some problems that restrict their druggability, such as low activity, overly high dosages, overly fast metabolism, and presence of strong hepatic first pass effect. Therefore, there is still a need for the development of novel compounds with higher activity, better pharmacokinetic properties or capability of passing through the blood-brain barrier so as to further improve the therapeutic effect, better meet the clinical needs, cope with the large number of patients with brain metastases (about 40% of patients with non-small cell lung cancer will develop brain metastases, but the existing clinical candidate compounds including MRTX849 and AMG-510 are not for these patients), and benefit more cancer patients.

### SUMMARY

### Problems to be Solved Herein

The present invention aims to provide a novel compound with inhibitory effect on KRAS G12C protein, a preparation method thereof, a pharmaceutical composition comprising the same, and pharmaceutical use thereof.

### Solutions for Solving the Problems

In a first aspect, the present invention provides a compound of formula I:

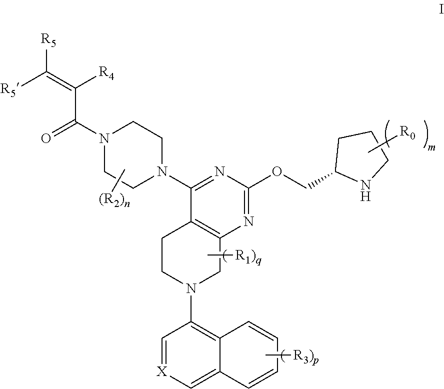

or a pharmaceutically acceptable salt, a solvate, a hydrate, a stereoisomer, a tautomer, an isotopically labeled compound or a prodrug thereof or a mixture thereof in any proportion, wherein,

### X is —CR₆═ or —N═;

each R₀ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, heteroalkyl, alkenyl, alkynyl, cycloalkyl, heterocycloalkyl, cyano, amino, haloalkyl or haloalkoxy, and the hydrogen in the R₁ structure is optionally substituted with 0 or more R₇;
each R₂ is independently hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl, or haloalkoxy;
each R₃ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, and the hydrogen in the R₃ structure is optionally substituted with 0 or more R₇;
R₄, R₅ and R_{5′} are each independently hydrogen, deuterium, halogen, alkyl, alkenyl, alkynyl, cycloalkyl, heterocycloalkyl, alkylaminoalkyl, cycloalkylaminoalkyl, amino, hydroxy, or haloalkyl, and the hydrogen in the R₄, R₅ and R_{5′} structures is optionally substituted with 1 or more substituents, wherein each of the substituents is independently deuterium, halogen, amino, hydroxy, alkoxy, alkanoyl, alkanoyloxy, alkoxycarbonyl, alkylsulfonamido, or cyano;
R₆ is hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl, or haloalkoxy;
each R₇ is independently hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl, or haloalkoxy;
m, n, p and q are each independently 0, 1 or 2.

In a second aspect, the present invention provides the compound of formula I described above selected from:
(1) 2-((S)-1-acryloyl-4-(7-(8-(methyl-d₃)naphthalen-1-yl)-2-(((S)-1-methylpyrrolidin-2-yl)methoxy)-5,6,7,8-tetrahydropyrido [3,4-d]pyrimidin-4-yl)piperazin-2-yl)acetonitrile;
(2) 2-((S)-1-(2-fluoroacryloyl)-4-(7-(8-(methyl-d₃)naphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile;
(3) (E)-4-(cyclopropylamino)-1-((S)-2-methyl-4-(7-(8-(methyl-d₃)naphthalen-1-yl)-2-(((S)-1-methylpyrrolidin-2-yl)methoxy)-5,6,7,8-tetrahydropyrido[3,4-d]pyrimidin-4-yl)piperazin-1-yl)-2-buten-1-one;
(4) 2-((S)-1-acryloyl-4-(7-(5-(methyl-d₃)isoquinolin-4-yl)-2-(((S)-1-methylpyrrolidin-2-yl)methoxy)-5,6,7,8-tetrahydropyrido [3,4-c]pyrimidin-4-yl)piperazin-2-yl)acetonitrile;
(5) 2-fluoro-1-((S)-2-methyl-4-(7-(8-(methyl-d₃)naphthalen-1-yl)-2-(((S)-1-methylpyrrolidin-2-yl)methoxy)-5,6,7,8-tetrahydropyrido[3,4-c]pyrimidin-4-yl)piperazin-1-yl)-2-propen-1-one;
(6) 2-((S)-1-acryloyl-4-(7-(8-(methyl-¹¹C)naphthalen-1-yl)-2-(((S)-1-methylpyrrolidin-2-yl)methoxy)-5,6,7,8-tetrahydropyrido [3,4-c]pyrimidin-4-yl)piperazin-2-yl)acetonitrile;
(7) 2-fluoro-1-((S)-3-methyl-4-(7-(8-(methyl-d₃)naphthalen-1-yl)-2-(((S)-1-methylpyrrolidin-2-yl)methoxy)-5,6,7,8-tetrahydropyrido[3,4-c]pyrimidin-4-yl)piperazin-1-yl)-2-propen-1-one;
(8) 1-((S)-3-methyl-4-(7-(8-(methyl-d₃)naphthalen-1-yl)-2-(((S)-1-methylpyrrolidin-2-yl)methoxy)-5,6,7,8-tetrahydropyrido [3,4-c]pyrimidin-4-yl)piperazin-1-yl)-2-propen-1-one;
(9) 2-((S)-1-(2-fluoroacryloyl)-4-(7-(8-(methyl-¹¹C)naphthalen-1-yl)-2-(((S)-1-methylpyrrolidin-2-yl)methoxy)-5,6,7,8-tetrahydropyrido[3,4-c]pyrimidin-4-yl)piperazin-2-yl)acetonitrile;
(10) 2-((S)-1-acryloyl-4-(7-(8-(methyl-¹³C)naphthalen-1-yl)-2-(((S)-1-methylpyrrolidin-2-yl)methoxy)-5,6,7,8-tetrahydropyrido [3,4-c]pyrimidin-4-yl)piperazin-2-yl)acetonitrile;
(11) 2-((S)-1-(2-fluoroacryloyl)-4-(7-(8-(methyl-¹³C)naphthalen-1-yl)-2-(((S)-1-methylpyrrolidin-2-yl)methoxy)-5,6,7,8-tetrahydropyrido[3,4-c]pyrimidin-4-yl)piperazin-2-yl)acetonitrile;
(12) 2-((S)-1-acryloyl-4-(7-(8-(methyl-¹⁴C)naphthalen-1-yl)-2-(((S)-1-methylpyrrolidin-2-yl)methoxy)-5,6,7,8-tetrahydropyrido [3,4-c]pyrimidin-4-yl)piperazin-2-yl)acetonitrile;
(13) 2-((S)-1-(2-fluoroacryloyl)-4-(7-(8-(methyl-¹⁴C)naphthalen-1-yl)-2-(((S)-1-methylpyrrolidin-2-yl)methoxy)-5,6,7,8-tetrahydropyrido[3,4-c]pyrimidin-4-yl)piperazin-2-yl)acetonitrile;
(14) 2-((S)-1-(2-fluoroacryloyl)-4-(7-(5-(methyl-¹⁴C)isoquinolin-4-yl)-2-(((S)-1-methylpyrrolidin-2-yl)methoxy)-5,6,7,8-tetrahydropyrido[3,4-d]pyrimidin-4-yl)piperazin-2-yl)acetonitrile;
(15) 2-((S)-1-acryloyl-4-(7-(5-(methyl-¹³C)isoquinolin-4-yl)-2-(((S)-1-methylpyrrolidin-2-yl)methoxy)-5,6,7,8-tetrahydropyrido [3,4-c]pyrimidin-4-yl)piperazin-2-yl)acetonitrile;
(16) 2-((S)-1-(2-fluoroacryloyl)-4-(7-(5-(methyl-¹³C)isoquinolin-4-yl)-2-(((S)-1-methylpyrrolidin-2-yl)methoxy)-5,6,7,8-tetrahydropyrido[3,4-d]pyrimidin-4-yl)piperazin-2-yl)acetonitrile;
(17) 2-((S)-1-acryloyl-4-(7-(8-(methyl-d₃)naphthalen-1-yl)-2-(((S)-1-(methyl-d₃)pyrrolidin-2-yl)methoxy)-5,6,7,8-tetrahydropyrido[3,4-d]pyrimidin-4-yl)piperazin-2-yl)acetonitrile;
(18) 2-((S)-4-(7-(8-chloronaphthalen-1-yl)-2-(((S)-1-(methyl-d₃)pyrrolidin-2-yl)methoxy)-5,6,7,8-tetrahydropyrido[3,4-d] pyrimidin-4-yl)-1-(2-fluoroacryl-oyl)piperazin-2-yl)acetonitrile;
(19) 2-((S)-1-((E)-4-(cyclopropylamino)-2-butenoyl)-4-(2-(((S)-1-(methyl-d₃)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile;
(20) 2-((S)-1-acryloyl-4-(7-(5-chloroisoquinolin-4-yl)-2-(((S)-1-(methyl-d₃)pyrrolidin-2-yl)methoxy)-5,6,7,8-tetrahydropyrido [3,4-d]pyrimidin-4-yl)piperazin-2-yl)acetonitrile;
(21) 1-((S)-2-methyl-4-(7-(8-(methyl-d₃)naphthalen-1-yl)-2-(((S)-1-(methyl-d₃)pyrrolidin-2-yl) methoxy)-5,6,7,8-tetrahydropyrido[3,4-d]pyrimidin-4-yl)piperazin-1-yl)-2-propen-1-one;
(22) 2-((S)-1-acryloyl-4-(2-(((S)-1-(methyl-d₃)pyrrolidin-2-yl)methoxy)-7-(8-(methyl-¹¹C)naphthalen-1-yl)-5,6,7,8-tetrahydropyrido[3,4-d]pyrimidin-4-yl)piperazin-2-yl)acetonitrile;
(23) 2-fluoro-1-((S)-3-methyl-4-(7-(8-(methyl-d₃)naphthalen-1-yl)-2-(((S)-1-(methyl-d₃)pyrrolidin-2-yl)methoxy)-5,6,7,8-tetrahydropyrido[3,4-d]pyrimidin-4-yl)piperazin-1-yl)-2-propen-1-one;
(24) 1-((S)-3-methyl-4-(7-(8-(methyl-d₃)naphthalen-1-yl)-2-(((S)-1-(methyl-d₃)pyrrolidin-2-yl) methoxy)-5,6,7,8-tetrahydropyrido[3,4-d]pyrimidin-4-yl)piperazin-1-yl)-2-propen-1-one;
(25) 2-((S)-1-acryloyl-4-(2-(((S)-1-(methyl-¹¹C)pyrrolidin-2-yl)methoxy)-7-(8-methylnaphthalen-1-yl)-5,6,7,8-tetrahydropyrido [3,4-d]pyrimidin-4-yl)piperazin-2-yl)acetonitrile;
(26) 2-((S)-1-acryloyl-4-(2-(((S)-1-(methyl-¹³C)pyrrolidin-2-yl)methoxy)-7-(8-methylnaphthalen-1-yl)-5,6,7,8-tetrahydropyrido [3,4-d]pyrimidin-4-yl)piperazin-2-yl)acetonitrile;
(27) 2-(S)-1-acryloyl-4-(7-(8-chloronaphthalen-1-yl)-2-(((S)-1-(methyl-¹¹C)pyrrolidin-2-yl)methoxy)-5,6,7,8-tetrahydropyrido [3,4-d]pyrimidin-4-yl)piperazin-2-yl)acetonitrile;
(28) 2-((S)-1-acryloyl-4-(7-(8-chloronaphthalen-1-yl)-2-(((S)-1-(methyl-¹³C)pyrrolidin-2-yl)methoxy)-5,6,7,8-tetrahydropyrido [3,4-d]pyrimidin-4-yl)piperazin-2-yl)acetonitrile;
(29) 2-(S)-1-(2-fluoroacryloyl)-4-(2-(((S)-1-(methyl-d₃)pyrrolidin-2-yl)methoxy)-7-(8-(methyl-¹⁴C)naphthalen-1-yl)-5,6,7,8-tetrahydropyrido[3,4-d]pyrimidin-4-yl)piperazin-2-yl)acetonitrile;
(30) 2-((S)-1-acryloyl-4-(2-(((S)-1-(methyl-d₃)pyrrolidin-2-yl)methoxy)-7-(5-methylisoquinolin-4-yl)-5,6,7,8-tetrahydropyrido [3,4-d]pyrimidin-4-yl)piperazin-2-yl)acetonitrile;
(31) 2-((S)-1-acryloyl-4-(2-(((S)-1-(methyl-d₃)pyrrolidin-2-yl)methoxy)-7-(5-(methyl-¹³C)isoquinolin-4-yl)-5,6,7,8-tetrahydropyrido[3,4-d]pyrimidin-4-yl)piperazin-2-yl)acetonitrile;
(32) 2-((S)-1-acryloyl-4-(2-(((S)-1-(methyl-d₃)pyrrolidin-2-yl)methoxy)-7-(5-(methyl-¹⁴C)isoquinolin-4-yl)-5,6,7,8-tetrahydropyrido[3,4-d]pyrimidin-4-yl)piperazin-2-yl)acetonitrile;
(33) 2-((S)-1-acryloyl-4-(2-(((S)-1-(ethyl-2,2,2-d₃)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile;
(34) 2-((S)-1-acryloyl-4-(7-(8-chloronaphthalen-1-yl)-2-(((S)-1-(ethyl-2,2,2-d₃)pyrrolidin-2-yl)methoxy)-5,6,7,8-tetrahydropyrido[3,4-d]pyrimidin-4-yl)piperazin-2-yl)acetonitrile;
(35) 2-((S)-1-acryloyl-4-(2-(((S)-1-(ethyl-d₅)pyrrolidin-2-yl)methoxy)-7-(8-methylnaphthalen-1-yl)-5,6,7,8-tetrahydropyrido [3,4-d]pyrimidin-4-yl)piperazin-2-yl)acetonitrile;
(36) 2-(S)-1-acryloyl-4-(7-(8-chloronaphthalen-1-yl)-2-(((S)-1-(ethyl-d₅)pyrrolidin-2-yl)methoxy)-5,6,7,8-tetrahydropyrido [3,4-d]pyrimidin-4-yl)piperazin-2-yl)acetonitrile;
(37) 2-((S)-1-acryloyl-4-(7-(8-methylnaphthalen-1-yl)-2-(((S)-1-(isopropyl-d₇)pyrrolidin-2-yl)methoxy)-5,6,7,8-tetrahydropyrido [3,4-d]pyrimidin-4-yl)piperazin-2-yl)acetonitrile;
(38) 2-(S)-1-acryloyl-4-(7-(8-chloronaphthalen-1-yl)-2-(((S)-1-(isopropyl-d₇)pyrrolidin-2-yl)methoxy)-5,6,7,8-tetrahydropyrido [3,4-d]pyrimidin-4-yl)piperazin-2-yl)acetonitrile;
(39) 2-(S)-1-acryloyl-4-(2-(((S)-1-(methyl-¹⁴C)pyrrolidin-2-yl)methoxy)-7-(8-methylnaphthalen-1-yl)-5,6,7,8-tetrahydropyrido [3,4-d]pyrimidin-4-yl)piperazin-2-yl)acetonitrile;
(40) 2-(S)-1-acryloyl-4-(7-(8-chloronaphthalen-1-yl)-2-(((S)-1-(methyl-¹⁴C)pyrrolidin-2-yl)methoxy)-5,6,7,8-tetrahydropyrido [3,4-d]pyrimidin-4-yl)piperazin-2-yl)acetonitrile;
(41) 2-((S)-1-acryloyl-4-(7-(8-chloronaphthalen-1-yl)-2-(((S)-1-(methyl-d₃)pyrrolidin-2-yl)methoxy)-5,6,7,8-tetrahydropyrido [3,4-d]pyrimidin-4-yl)piperazin-2-yl)acetonitrile; and
(42) 2-((S)-1-acryloyl-4-(7-(8-methylnaphthalen-1-yl)-2-(((S)-1-(methyl-d₃)pyrrolidin-2-yl)methoxy)-5,6,7,8-tetrahydropyrido
[3,4-d]pyrimidin-4-yl)piperazin-2-yl)acetonitrile.

In a third aspect, the present invention provides a method for preparing the compound of formula I described above comprising:

1) reacting compound I-1 with compound I-2 to obtain compound I-3;

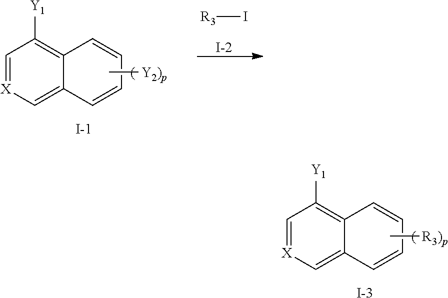

2) reacting compound I-3 with compound I-4 to obtain compound I-5;

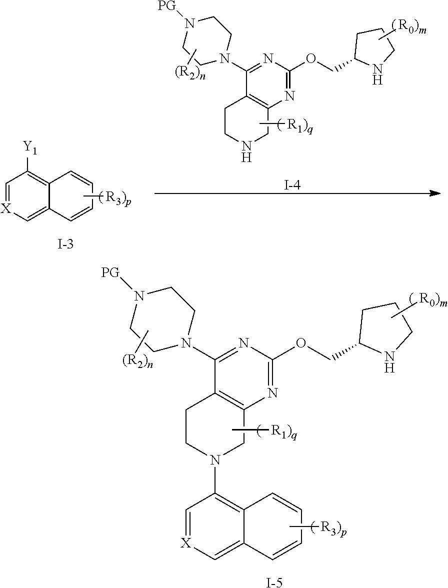

3) subjecting compound I-5 to deprotection reaction to obtain compound I-6; and

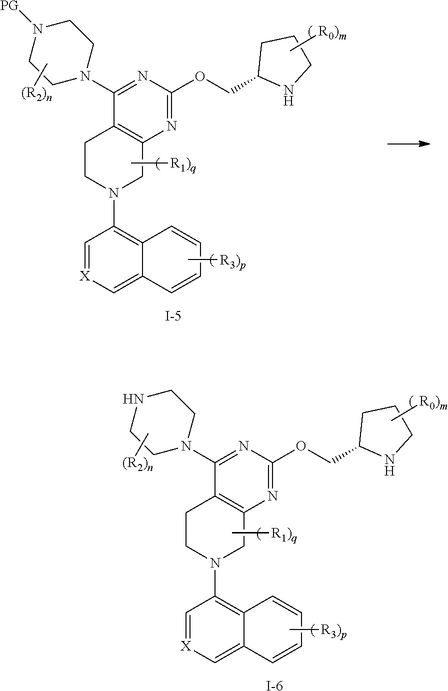

4) reacting compound I-6 with compound I-7 to obtain the compound of formula I;

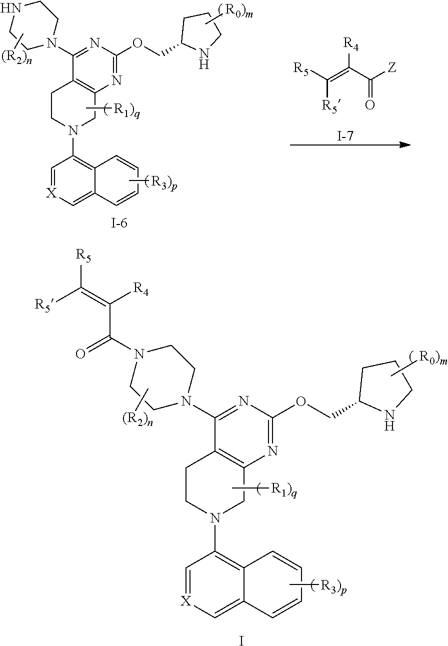

wherein Y₁ and Y₂ are each independently chlorine, bromine, iodine, methanesulfonyloxy, trifluoromethanesulfonyloxy, p-toluenesulfonyloxy, a borate ester, a zinc halide group, a magnesium halide group, or a tin halide group; z is hydroxy, bromine or chlorine; PG represents a protecting group; x, R₀, R₁, R₂, R₃, R₄, R₅, R_{5′}, m, n, p and q are as defined in formula I.

In a fourth aspect, the present invention provides a pharmaceutical composition comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, and a pharmaceutically acceptable carrier.

In a fifth aspect, the present invention provides a contrast agent composition comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, and a pharmaceutically acceptable carrier.

In a sixth aspect, the present invention provides a tracer composition comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, and a pharmaceutically acceptable carrier.

In a seventh aspect, the present invention provides the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition, the contrast agent or the tracer composition described above, for use as a KRAS G12C protein inhibitor.

In an eighth aspect, the present application provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition described above, in preparing a medicament for preventing and/or treating a disease at least partially mediated by KRAS G12C protein.

In a ninth aspect, the present invention provides a method for preventing and/or treating a disease at least partially mediated by KRAS G12C protein comprising: administering to an individual in need thereof a therapeutically effective amount of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion, or the pharmaceutical composition described above.

In a tenth aspect, the present invention provides a pharmaceutical combination form comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition described above, and at least one additional therapeutic agent for cancer.

In an eleventh aspect, the present invention provides a method for preventing and/or treating cancer comprising: administering to an individual in need thereof a therapeutically effective amount of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition described above, or the pharmaceutical combination form described above.

In a twelfth aspect, the present application provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the contrast agent composition described above, in preparing a contrast agent kit for diagnosing a disease at least partially mediated by KRAS G12C protein.

In a thirteenth aspect, the present application provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the tracer composition described above, in preparing a tracer kit for diagnosing a disease at least partially mediated by KRAS G12C protein.

### Beneficial Effects

The present invention provides a compound of formula I with a novel structure, which can be used as a high-efficiency KRAS G12C protein inhibitor and has various pharmacological activities against tumors, proliferative diseases, inflammation, autoimmune diseases, etc.

### BRIEF DESCRIPTION OF THE DRAWING

FIG. 1 shows tumor growth inhibition curves of test compounds against nude mouse NCI-H358.

### DETAILED DESCRIPTION

Before the present invention is further described, it is to be understood that the present invention is not limited to the particular embodiments described herein; it is also to be understood that the terminology used herein is for the purpose of describing the particular embodiments only and is not intended to be limiting.

### Definitions of Terms

Unless otherwise stated, the following terms have the following meanings.

The “pharmaceutically acceptable salt” refers to a salt of the compound of formula I that is substantially non-toxic to organisms. Pharmaceutically acceptable salts generally include, but are not limited to, salts formed by reacting the compounds of the present invention with a pharmaceutically acceptable inorganic/organic acid or inorganic/organic base. Such salts are also referred to as acid addition salts or base addition salts. Common inorganic acids include, but are not limited to, hydrochloric acid, hydrobromic acid, sulfuric acid, phosphoric acid, etc. Common organic acids include, but are not limited to, trifluoroacetic acid, citric acid, maleic acid, fumaric acid, succinic acid, tartaric acid, lactic acid, pyruvic acid, oxalic acid, formic acid, acetic acid, benzoic acid, methanesulfonic acid, benzenesulfonic acid, p-toluenesulfonic acid, etc. Common inorganic bases include, but are not limited to, sodium hydroxide, potassium hydroxide, calcium hydroxide, barium hydroxide, etc. Common organic bases include, but are not limited to, diethylamine, triethylamine, ethambutol, etc.

The term “solvate” refers to a substance formed by the binding of the compounds of the present invention or the pharmaceutically acceptable salt thereof to at least one solvent molecule through non-covalent intermolecular forces. The term “solvate” includes “hydrate”. Common solvates include, but are not limited to, hydrates, ethanolates, acetonates, etc.

The term “hydrate” refers to a substance formed by the binding of the compounds of the present invention or the pharmaceutically acceptable salt thereof to water through non-covalent intermolecular forces. Common hydrates include, but are not limited to, hemihydrate, monohydrate, dihydrate, trihydrate, etc.

The term “isomer” refers to a compound having the same number and type of atoms, and thus the same molecular weight, but differing in the spatial arrangement or configuration of the atoms.

The term “stereoisomer” refers to an isomer resulting from different spatial arrangements of the atoms in a molecule, and includes both “configurational isomers” and “conformational isomers”. The term “configurational isomers” refers to isomers resulting from different spatial arrangements of the atoms in a molecule, and includes both “cis-trans isomers” and “optical isomers”. The term “cis-trans isomers” refers to isomers resulting from different positions of the atoms on both sides of a double bond or a ring system relative to a reference plane. In a cis isomer, the atoms (or groups) are on the same side of a double bond or a ring system, while in a trans isomer, the atoms (or groups) are on opposing sides of a double bond or a ring system, wherein the “double bond” refers generally to a carbon-carbon double bond, and also includes a carbon-nitrogen double bond and a nitrogen-nitrogen double bond. The term “optical isomers” refers to stable isomers that have perpendicular asymmetric planes due to having at least one chiral factor (including chiral center, chiral axis, chiral plane, etc.) so that they are able to rotate plane-polarized light. Due to the presence of asymmetric centers and other chemical structures in the compounds of the present invention that may lead to stereoisomers, these stereoisomers and mixtures thereof are also encompassed by the present invention. Since the compounds of the present invention and the salts thereof contain asymmetric carbon atoms, they can exist in the forms of single stereoisomers, racemates, and mixtures of enantiomers and diastereomers. Generally, these compounds can be prepared in the form of a racemic mixture. However, if desired, such compounds may be prepared or isolated to obtain pure stereoisomers, i.e., single enantiomers or diastereomers, or mixtures enriched in single stereoisomers (purity ≥98%, ≥95%, ≥93%, ≥90%, ≥88%, ≥85% or ≥80%). As described hereinafter, single stereoisomers of a compound are prepared synthetically from optically active starting materials containing the desired chiral center, or obtained by preparation of a mixture of enantiomeric products followed by isolation or resolution, e.g., conversion to a mixture of diastereomers followed by isolation or recrystallization, chromatography, use of chiral resolving reagents, or direct isolation of the enantiomers on a chiral chromatography column. Starting compounds of a particular stereochemistry are either commercially available or may be obtained by preparation according to the methods described hereinafter followed by resolution using methods well known in the art. The term “enantiomer” refers to a pair of stereoisomers that have non-overlapping mirror images of each other.

The term “diastereomer” or “diastereoisomer” refers to optical isomers that do not form mirror images of each another. The term “racemic mixture” or “racemate” refers to a mixture containing equal parts of single enantiomers (i.e., an equimolar mixture of both of R and S enantiomers). The term “non-racemic mixture” refers to a mixture containing unequal parts of single enantiomers. Unless otherwise indicated, all stereoisomeric forms of the compounds of the present invention fall within the scope of the present invention.

The term “tautomer” (or “tautomeric form”) refers to structural isomers having different energies that can interconvert via a low energy barrier. If tautomers are possible (e.g., in solution), the chemical equilibrium of the tautomers can be achieved. For example, proton tautomers (or prototropic tautomers) interconverts in a manner including, but not limited to, proton migration, such as keto-enol isomerization, imine-enamine isomerization, and amide-iminoalcohol isomerization. Unless otherwise indicated, all tautomeric forms of the compounds of the present invention fall within the scope of the present invention.

The term “isotopically labeled compound” refers to a compound formed by replacing a particular atom in a structure with its isotopic atom. Unless otherwise indicated, the compounds of the present invention contain various isotopes of H, C, N, O, F, P, S and Cl, such as ²H(D), ³H(T), ¹¹C, ¹³C, ¹⁴C, ¹⁵N, ¹⁷O, ¹⁸O, ¹⁸F, ³¹P, ³²P, ³⁵S, ³⁶S and ³⁷Cl.

The term “prodrug” refers to a derivative compound that, when administered to an individual, is capable of providing, directly or indirectly, the compound of the present invention. Particularly preferred derivative compounds or prodrugs are compounds that, when administered to an individual, can increase the bioavailability of the compounds of the present invention (e.g., it is more readily absorbed into the blood), or facilitate delivery of the parent compound to a site of action (e.g., the lymphatic system). Unless otherwise indicated, all prodrug forms of the compounds of the present invention fall within the scope of the present invention, and are well known in the art.

The term “each independently” means that at least two groups (or ring systems) present in the structure having the same or similar range of values may have the same or different meaning under a particular circumstance. For example, X and Y are each independently hydrogen, halogen, hydroxy, cyano, alkyl or aryl, meaning that when X is hydrogen, Y may be hydrogen or halogen, hydroxy, cyano, alkyl or aryl; similarly, when Y is hydrogen, X may be hydrogen or halogen, hydroxy, cyano, alkyl or aryl.

The term “optional” or “optionally” means that the subsequently described event or circumstance may or may not occur. The description includes instances where the event or circumstance occurs and instances where it does not. For example, ethyl is “optionally” substituted with halogen, meaning that the ethyl group may be unsubstituted (CH₂CH₃), monosubstituted (e.g., CH₂CH₂F), polysubstituted (e.g., CHFCH₂F, and CH₂CHF₂), or fully substituted (CF₂CF₃); 5-10 membered aryl or heteroaryl is “optionally” substituted with 1 to 3 R, meaning that the 5-10 membered aryl or heteroaryl may be unsubstituted, or may be substituted with 1 to 3 R. It will be understood by those skilled in the art that for any group comprising one or more substituents, no substituent or substitution pattern that is spatially impossible or cannot be synthesized will be introduced.

The term “1,8-disubstituted” means that in a fused bicyclic ring system consisting of two six membered rings (e.g., a naphthalene ring, a tetrahydronaphthalene ring, a quinoline ring, an isoquinoline ring, and a benzotetrahydropyridine ring), when two or more substituents are present, two of the substituents are linked to ring atoms at α-positions on the same side of the different rings. Accordingly, “1,4-disubstituted” corresponds to the substitution on opposing sides of the same ring, and “1,5-disubstituted” corresponds to the substitution on opposing sides of the different rings. It can be understood that “1,8-disubstituted” is used only to illustrate the substitution pattern of two substituents on the same side of different rings and is not intended to limit the linking of the two substituents to positions 1 and 8 of the ring system since the numbering order of the ring system will vary with the presence of heteroatoms. For example, when substituents are linked to 2 ring atoms on the quinoline ring at a positions on the same side of the different rings at the same time, although the two ring atoms are at positions 4 and 5, respectively, it is customary in the art to treat the two substituents linked thereto as being present in the “1,8-disubstituted” form rather than in the “4,5-disubstituted” form.

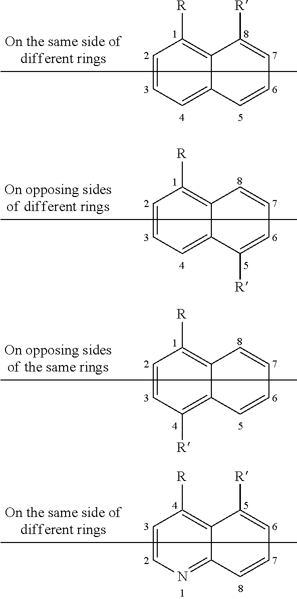

The term “halogen” refers to fluorine (F), chlorine (Cl), bromine (Br) and iodine (I) in group VIIA of the periodic table of the elements, preferably fluorine, chlorine and bromine, and more preferably fluorine and chlorine.

The term “cyano” refers to the monovalent group —CN.

The term “hydroxy” refers to the monovalent group —OH.

The term “amino” refers to the monovalent group —NH₂, wherein two hydrogen atoms are optionally substituted with substituents described in the present invention.

The term “carbonyl” refers to the divalent group —C(═O)—.

The term “alkyl” refers to a monovalent linear or branched hydrocarbyl group that consists of carbon and hydrogen atoms only, has no degree of unsaturation and links to a core or another group through a single bond, preferably C₁₋₆ alkyl, and more preferably C₁₋₄ alkyl; common alkyl groups include, but are not limited to, methyl (—CH₃), ethyl (—CH₂CH₃), n-propyl (—CH₂CH₂CH₃), isopropyl (—CH(CH₃)₂), n-butyl (—CH₂CH₂CH₂CH₃), sec-butyl (—CH(CH₃)CH₂CH₃), isobutyl (—CH₂CH(CH₃)₂), tert-butyl (—C(CH₃)₃), n-pentyl (—CH₂CH₂CH₂CH₂CH₃), neopentyl (—CH₂C(CH₃)₃), etc.

The term “heteroalkyl” refers to a monovalent linear or branched group whose branch atoms consist of carbon atoms and heteroatoms selected from nitrogen, oxygen, sulfur and phosphorus, which has no degree of unsaturation and links to a core or another group through a single bond; common heteroalkyl groups include, but are not limited to, methoxymethyl (MOM, —CH₂OCH₃), methoxyethyl (MOE, —CH₂CH₂OCH₃), methoxypropyl (MOP, —CH₂CH₂CH₂OCH₃), methylthiomethyl (MTM, —CH₂SCH₃), methylthioethyl (MTE, —CH₂CH₂SCH₃), methylaminomethyl (MAM, —CH₂NHCH₃), dimethylaminomethyl (DMAM, —CH₂N(CH₃)₂), etc. The terms “alkoxyalkyl”, “alkylthioalkyl” and “alkylaminoalkyl” are specific forms of the term “heteroalkyl”.

The term “haloalkyl” refers to a monovalent linear or branched alkyl group that contains at least one halogen atom, has no degree of unsaturation and links to a core or another group through a single bond, preferably C₁₋₆ haloalkyl, and more preferably C₁₋₄ haloalkyl; common haloalkyl groups include, but are not limited to, fluoromethyl (—CH₂F), difluoromethyl (—CHF₂), trifluoromethyl (—CF₃), 1,2-difluoroethyl (—CHFCH₂F), 2,2,2-trifluoroethyl (—CH₂CF₃), chloromethyl (—CH₂C₁), dichloromethyl (—CHCl₂), trichloromethyl (—CCl₃), etc.

The term “cyanoalkyl” refers to a monovalent linear or branched alkyl group that contains at least one cyano group, has no degree of unsaturation other than that in cyano and links to a core or another group through a single bond, preferably C₁₋₆ cyanoalkyl, and more preferably C₁₋₄ cyanoalkyl; common cyanoalkyl groups include, but are not limited to, cyanomethyl (—CH₂CN), dicyanomethyl (—CH(CN)₂), 1-cyanoethyl (—CH(CN)CH₃), 2-cyanoethyl (—CH₂CH₂CN), etc.

The term “alkenyl” refers to a monovalent linear or branched chain hydrocarbyl group that consists of carbon and hydrogen atoms only, contains at least one carbon-carbon double bond and links to a core or another group through a single bond linked to a double bond, preferably C₂₋₆ alkenyl, and more preferably C₂₋₄ alkenyl; common alkenyl groups include, but are not limited to, vinyl (—CH═CH₂), 1-propen-1-yl (—CH═CH—CH₃), 1-buten-1-yl (—CH═CH—CH₂—CH₃), 1-penten-1-yl (—CH═CH—CH₂—CH₂—CH₃), 1,3-butadien-1-yl (—CH═CH—CH═CH₂), 1,4-pentadien-1-yl (—CH═CH—CH₂—CH═CH₂), etc.

The term “alkynyl” refers to a monovalent linear or branched chain hydrocarbyl group that consists of carbon and hydrogen atoms only, contains at least one carbon-carbon triple bond and links to a core or another group through a single bond linked to a triple bond, preferably C₂₋₆ alkynyl, and more preferably C₂₋₄ alkynyl; common alkynyl groups include, but are not limited to, ethynyl (—C≡CH), 1-propyn-1-yl (i.e., propynyl) (—C≡C—CH₃) 1-butyn-1-yl (i.e., butynyl)

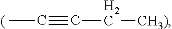

pentyn-1-yl

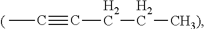

1,3-butadiyn-1-yl (—C≡C—C≡CH), 1,4-pentadiyn-1-yl

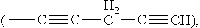

etc.

The term “alkoxy” refers to a monovalent linear or branched group that consists of carbon, hydrogen and oxygen atoms only, has no degree of unsaturation and links to a core or another group through a single bond linked to the oxygen atom, preferably C₁₋₆ alkoxy, and more preferably C₁₋₄ alkoxy; common alkoxy groups include, but are not limited to, methoxy (—OCH₃), ethoxy (—OCH₂CH₃), n-propoxy (—OCH₂CH₂CH₃), isopropoxy (—OCH(CH₃)₂), n-butoxy (—OCH₂CH₂CH₂CH₃), sec-butoxy (—OCH(CH₃)CH₂CH₃), isobutoxy (—OCH₂CH(CH₃)₂), tert-butoxy (—OC(CH₃)₃), n-pentoxy (—OCH₂CH₂CH₂CH₂CH₃), neopentoxy (—OCH₂C(CH₃)₃), etc.

The term “haloalkoxy” refers to a monovalent linear or branched alkoxy group that contains at least one halogen atom, has no degree of unsaturation and links to a core or another group through a single bond linked to the oxygen atom, preferably C₁₋₆ haloalkoxy, and more preferably C₁₋₄ haloalkoxy; common haloalkoxy groups include, but are not limited to, fluoromethoxy (—OCH₂F), difluoromethoxy (—OCHF₂), trifluoromethoxy (—OCF₃), 2,2,2-trifluoroethoxy (—OCH₂CF₃), pentafluoroethoxy (—OCF₂CF₃), etc.

The term “alkylamino” refers to a monovalent linear or branched group that consists of carbon, hydrogen and nitrogen atoms only, has no degree of unsaturation and links to a core or another group through a single bond linked to the nitrogen atom, preferably C₁₋₆ alkylamino, and more preferably C₁₋₄ alkylamino; common alkylamino groups include, but are not limited to, methylamino (—NHCH₃), dimethylamino (—N(CH₃)₂), ethylamino (—NHCH₂CH₃), diethylamino (—N(CH₂CH₃)₂), n-propylamino (—NHCH₂CH₂CH₃), isopropylamino (—NHCH(CH₃)₂), etc.

The term “alkanoyl” refers to a monovalent linear or branched group that is formed by the linking of alkyl to carbonyl, has no degree of unsaturation other than that in the carbonyl and links to a core or another group through a single bond linked to the carbonyl group; common alkanoyl groups include, but are not limited to, formyl (—C(═O)H), acetyl (—C(═O)CH₃), n-propionyl (—C(═O)CH₂CH₃), n-butyryl (—C(═O)CH₂CH₂CH₃), isobutyryl (—C(═O)CH(CH₃)₂), n-valeryl (—C(═O)CH₂CH₂CH₂CH₃), pivaloyl (—C(═O)C(CH₃)₃), etc.

The term “alkanoyloxy” refers to a monovalent linear or branched group that is formed by the linking of alkanoyl to an oxygen atom, has no degree of unsaturation other than that in the carbonyl and links to a core or another group through a single bond linked to the oxygen atom; common alkylacyloxy groups include, but are not limited to, formyloxy (—OC(═O)H), acetyloxy (—OC(═O)CH₃), n-propionyloxy (—OC(═O)CH₂CH₃), n-butyryloxy (—OC(═O)CH₂CH₂CH₃), isobutyryloxy (—OC(═O)CH(CH₃)₂), n-valeryloxy (—OC(═O)CH₂CH₂CH₂CH₃), pivaloyloxy (—OC(═O)C(CH₃)₃), etc.

The term “alkoxycarbonyl” refers to a monovalent linear or branched group that is formed by the linking of alkoxy to carbonyl, has no degree of unsaturation other than that in the carbonyl and links to a core or another group through a single bond linked to the carbonyl; common alkoxycarbonyl groups include, but are not limited to, methoxycarbonyl (—C(═O)OCH₃), ethoxycarbonyl (—C(═O)OCH₂CH₃), n-propoxycarbonyl (—C(═O)OCH₂CH₂CH₃), isopropoxycarbonyl (—C(═O)OCH(CH₃)₂), n-butoxycarbonyl (—C(═O)OCH₂CH₂CH₂CH₃), tert-butoxycarbonyl (—C(═O)OC(CH₃)₃), etc.

The term “alkylsulfonamido” refers to a monovalent linear or branched group that is formed by the linking of alkyl to a divalent group —S(═O)NH—, has no degree of unsaturation other than that in the sulfoxide group (—S(═O)—) and links to a core or another group through a single bond linked to the nitrogen atom; common alkylsulfonamido groups include, but are not limited to, methanesulfonamido (—NHS(═O)CH₃), ethanesulfonamido (—NHS(═O)CH₂CH₃), etc.

The term “cycloalkyl” (or “alicyclic ring”) refers to a monovalent monocyclic, non-aromatic ring system that consists of carbon and hydrogen atoms only and links to a core or another group through a single bond, preferably C₃₋₈ cycloalkyl, and more preferably C₃₋₆ cycloalkyl; common cycloalkyl groups include, but are not limited to, cyclopropyl, cyclobutyl, cyclopentyl, cyclohexyl, cycloheptyl, cyclooctyl, decahydronaphthyl, adamantyl, etc.

The term “heterocycloalkyl” (or “heteroalicyclic ring”) refers to a monovalent monocyclic, nonaromatic ring system whose ring atoms consists of carbon atoms and heteroatoms selected from nitrogen, oxygen, sulfur and phosphorus, which links to a core or another group through a single bond, preferably 3-8 membered heterocycloalkyl, and more preferably 3-6 membered heterocycloalkyl; common heterocycloalkyl groups include, but are not limited to, oxiranyl, oxetan-3-yl, azetidin-3-yl, tetrahydrofuran-2-yl, pyrrolidin-1-yl, pyrrolidin-2-yl, tetrahydro-2H-pyran-2-yl, tetrahydro-2H-pyran-4-yl, piperidin-2-yl, piperidin-4-yl, morpholin-1-yl, etc.

The term “methyl-d₃” (or “deuterated methyl”) refers to the monovalent group —CD₃ obtained by replacing all of the hydrogen atoms (H) in the methyl with deuterium (D). Similarly, the term “ethyl-d₅” (or “deuterated ethyl”) refers to the monovalent group —CD₂CD₃; the term “isopropyl-d₇” (or “deuterated isopropyl”) refers to the monovalent group —CD(CD₃)₂.

The term “methyl-¹⁴C”, refers to the monovalent group —¹⁴CH₃ obtained by replacing ¹²C in the methyl with ¹⁴C. Similarly, the term “methyl-¹³C” refers to the monovalent radical —¹³CH₃; the term “methyl-¹¹C” refers to the monovalent group —¹¹CH₃.

“An embodiment”, “one embodiment”, “some embodiments”, “certain embodiments”, or “some embodiments” referred to in the specification means that a specific reference element, structure, or characteristic described in connection with the embodiment is included in at least one embodiment. Thus, the phrases “in one embodiment”, “in some embodiments”, “in another embodiments”, or “in certain embodiments” in various places throughout the specification are not necessarily all referring to the same embodiment. Furthermore, the specific elements, structures, or characteristics may be combined in any suitable manner in one or more embodiments.

The terms “comprise/contain/include/have” and the English variants thereof such as “comprises/contains/includes/has” and “comprising/containing/including/having” should be interpreted in an open-ended sense, i.e., “comprising/containing/including/having, but not limited to”.

It should be understood that, unless otherwise specified, the singular forms “a”, “an” and “the” referred to in the specification and the claims also include plural referents. For example, a reaction including a “catalyst” may include one catalyst, or two or more catalysts.

### [Compound of Formula]

The present invention provides a compound of formula I:

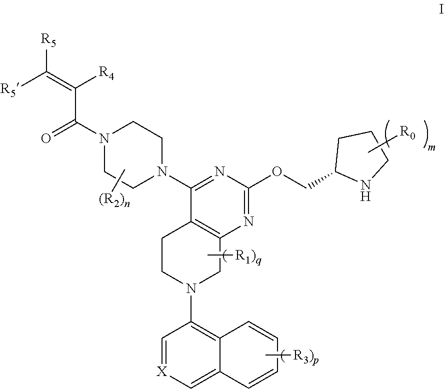

or a pharmaceutically acceptable salt, a solvate, a hydrate, a stereoisomer, a tautomer, an isotopically labeled compound or a prodrug thereof or a mixture thereof in any proportion, wherein,

### X is —CR₆═ or —N═;

each R₀ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, heteroalkyl, alkenyl, alkynyl, cycloalkyl, heterocycloalkyl, cyano, amino, haloalkyl or haloalkoxy, and the hydrogen in the R₁ structure is optionally substituted with 0 or more R₇;
each R₂ is independently hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl, or haloalkoxy;
each R₃ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, and the hydrogen in the R₃ structure is optionally substituted with 0 or more R₇;
R₄, R₅ and R_{5′} are each independently hydrogen, deuterium, halogen, alkyl, alkenyl, alkynyl, cycloalkyl, heterocycloalkyl, alkylaminoalkyl, cycloalkylaminoalkyl, amino, hydroxy, or haloalkyl, and the hydrogen in the R₄, R₅ and R_{5′} structures is optionally substituted with 1 or more substituents, wherein each of the substituents is independently deuterium, halogen, amino, hydroxy, alkoxy, alkanoyl, alkanoyloxy, alkoxycarbonyl, alkylsulfonamido, or cyano;
R₆ is hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl, or haloalkoxy;
each R₇ is independently hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl, or haloalkoxy;
m, n, p and q are each independently 0, 1 or 2.

In some preferred embodiments of the present invention, in the compound of formula I described above,

### X is —CR₆═ or —N═;

each R₀ is independently hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C, preferably alkyl, cycloalkyl, methyl-d₃, ethyl-d₅, ethyl-2,2,2-d₃, isopropyl-d₇, methyl-¹⁴C, methyl-¹³C or methyl-¹¹C, and more preferably alkyl, methyl-d₃, ethyl-d₅, ethyl-2,2,2-d₃, isopropyl-d₇, methyl-¹⁴C, methyl-¹³C or methyl-¹¹C, wherein the alkyl is preferably C₁-C₆ alkyl, more preferably methyl, ethyl or isopropyl, and most preferably methyl;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen; and the hydrogen in the R₁ structure is optionally substituted with 0 or more R₇;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl, wherein the alkyl is preferably C₁-C₆ alkyl, more preferably methyl, ethyl or isopropyl, and most preferably methyl; the cyanoalkyl is preferably C₂-C₆ cyanoalkyl, and more preferably cyanomethyl;
each R₃ is independently hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C, preferably halogen, alkyl, methyl-d₃, ethyl-d₅, ethyl-2,2,2-d₃, isopropyl-d₇, methyl-¹⁴C, methyl-¹³C, or methyl-¹¹C, and more preferably halogen, alkyl, methyl-d₃, methyl-¹⁴C, methyl-¹³C, or methyl-¹¹C, wherein the halogen is preferably fluorine, chlorine, bromine, or iodine, more preferably fluorine, chlorine, or bromine, and most preferably chlorine; the alkyl is preferably C₁-C₆ alkyl, more preferably methyl, ethyl, or isopropyl, and most preferably methyl; and the hydrogen in the R₃ structure is optionally substituted with 0 or more R₇;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl, wherein halogen is preferably fluorine, chlorine, bromine or iodine, more preferably fluorine, chlorine or bromine, and most preferably chlorine; the cycloalkylaminoalkyl is preferably (C₃-C₆ cycloalkyl)-NH—(C₁₋₆ alkylene)-, and more preferably cyclopropylaminomethyl; and the hydrogen in the R₄, R₅ and R_{5′} structures is optionally substituted with 1 or more substituents, wherein each of the substituents is independently deuterium, halogen, amino, hydroxy, alkoxy, alkanoyl, alkanoyloxy, alkoxycarbonyl, alkylsulfonamido, or cyano;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
each R₇ is each independently hydrogen, deuterium, halogen, alkyl, cycloalkyl, cyano, amino, haloalkyl or haloalkoxy, preferably hydrogen, deuterium, halogen, cyano or amino, and more preferably deuterium, halogen or cyano, wherein halogen is preferably fluorine, chlorine, bromine or iodine, more preferably fluorine, chlorine or bromine, and most preferably chlorine;
m, n, p and q are each independently 0, 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

In some embodiments of the present invention, the compound of formula I described above is a compound of formula I-A:

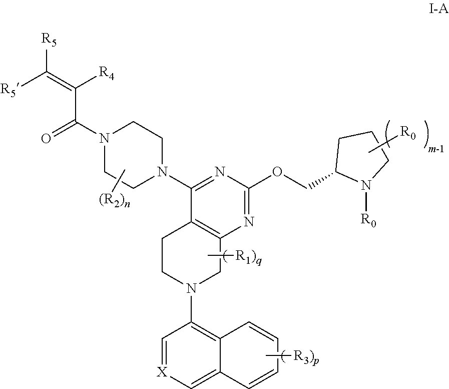

wherein X, R₀, R₁, R₂, R₃, R₄, R₅ or R_{5′} are as defined in formula I; m is 1 or 2; and n, p and q are each independently 0, 1 or 2.

In some preferred embodiments of the present invention, in the compound of formula I-A described above:

### X is —CR₆═ or —N═;

on the pyrrolidine ring, R₀ linked to the nitrogen atom is methyl-d₃, ethyl-d₅, ethyl-2,2,2-d₃, isopropyl-d₇, methyl-¹⁴C, methyl-¹³C or methyl-¹¹C; the remaining R₀ is hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₀ is hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, heteroalkyl, alkenyl, alkynyl, cycloalkyl, heterocycloalkyl, cyano, amino, haloalkyl or haloalkoxy, preferably hydrogen, deuterium, halogen, alkyl, cycloalkyl, cyano, amino, haloalkyl or haloalkoxy;
each R₂ is independently hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl or haloalkoxy, preferably hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy;
each R₃ is each independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, preferably hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C, and more preferably halogen, alkyl, methyl-d₃, methyl-¹⁴C, methyl-¹³C, or methyl-¹¹C;
R₄, R₅ and R_{5′} are each independently hydrogen, deuterium, halogen, alkyl, alkenyl, alkynyl, cycloalkyl, heterocycloalkyl, alkylaminoalkyl, cycloalkylaminoalkyl, amino, hydroxy or haloalkyl, preferably hydrogen, deuterium, halogen, alkyl, cycloalkyl, alkylaminoalkyl, cycloalkylaminoalkyl, amino or haloalkyl;
R₆ is hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl or haloalkoxy, preferably hydrogen, deuterium, halogen, alkyl, cycloalkyl, cyano, amino, haloalkyl or haloalkoxy;
m is 1 or 2, and n, p and q are each independently 0, 1 or 2.

In other preferred embodiments of the present invention, in the compound of formula I-A described above:

### X is —CR₆═ or —N═;

on the pyrrolidine ring, R₀ linked to the nitrogen atom is alkyl, methyl-d₃, ethyl-d₅, ethyl-2,2,2-d₃, isopropyl-d₇, methyl-¹⁴C, methyl-¹³C or methyl-¹¹C; the remaining R₀ is hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl ¹⁴C, methyl-¹³C or methyl ¹¹C; preferably, the remaining R₀ is hydrogen, alkyl, cycloalkyl, methyl-d₃, ethyl-d₅, ethyl-2,2,2-d₃, isopropyl-d₇, methyl ¹⁴C, methyl-¹³C or methyl-¹¹C; more preferably, the remaining R₀ is hydrogen, alkyl, methyl-d₃, ethyl-d₅, ethyl-2,2,2-d₃, isopropyl-d₇, methyl-¹⁴C, methyl-¹³C, or methyl-¹¹C, wherein the alkyl is preferably C₁-C₆ alkyl, more preferably methyl, ethyl, or isopropyl, and most preferably methyl;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl, wherein the alkyl is preferably C₁-C₆ alkyl, more preferably methyl, ethyl or isopropyl, and most preferably methyl; the cyanoalkyl is preferably C₂-C₆ cyanoalkyl, and more preferably cyanomethyl;
each R₃ is independently hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C, preferably halogen, alkyl, methyl-d₃, ethyl-d₅, ethyl-2,2,2-d₃, isopropyl-d₇, methyl-¹⁴C, methyl-¹³C, or methyl-¹¹C, and more preferably halogen, alkyl, methyl-d₃, methyl-¹⁴C, methyl-¹³C, or methyl-¹¹C, wherein the halogen is preferably fluorine, chlorine, bromine, or iodine, more preferably fluorine, chlorine, or bromine, and most preferably chlorine; the alkyl is preferably C₁-C₆ alkyl, more preferably methyl, ethyl, or isopropyl, and most preferably methyl;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl, wherein halogen is preferably fluorine, chlorine, bromine or iodine, more preferably fluorine, chlorine or bromine, and most preferably chlorine; the cycloalkylaminoalkyl is preferably (C₃-C₆ cycloalkyl)-NH—(C₁₋₆ alkylene)-, and more preferably cyclopropylaminomethyl;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
m is 1 or 2, and n, p and q are each independently 0, 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

In some embodiments of the present invention, the compound of formula I described above is a compound of formula I-B:

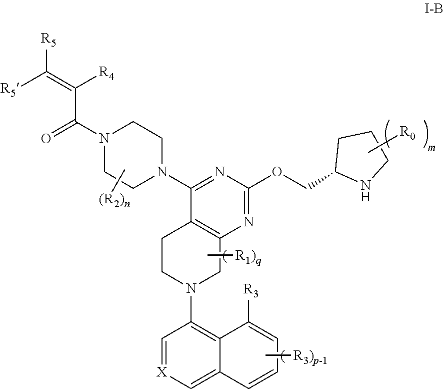

wherein X, R₀, R₁, R₂, R₃, R₄, R₅ or R_{5′} are as defined in formula I; m, n and q are each independently 0, 1 or 2; and p is 1 or 2.

In some preferred embodiments of the present invention, in the compound of formula I-B described above:

### X is —CR₆═ or —N═;

each R₀ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, preferably hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, and more preferably alkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, heteroalkyl, alkenyl, alkynyl, cycloalkyl, heterocycloalkyl, cyano, amino, haloalkyl or haloalkoxy, preferably hydrogen, deuterium, halogen, alkyl, cycloalkyl, cyano, amino, haloalkyl or haloalkoxy;
each R₂ is independently hydrogen, deuterium, halogen, alkyl, alkenyl, alkynyl, cycloalkyl, cyano, alkoxy, haloalkyl or haloalkoxy, preferably hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy;
on the aromatic ring containing X, R₃, together with the tetrahydropyridopyrimidine ring, present in the 1,8-disubstituted form is methyl-d₃, ethyl-d₅, ethyl-2,2,2-d₃, isopropyl-d₇, methyl-¹⁴C, methyl-¹³C or methyl-¹¹C; the remaining R₃ is hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₃ is hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C;
R₄, R₅ and R_{5′} are each independently hydrogen, deuterium, halogen, alkyl, alkenyl, alkynyl, cycloalkyl, heterocycloalkyl, alkylaminoalkyl, cycloalkylaminoalkyl, amino, hydroxy or haloalkyl, preferably hydrogen, deuterium, halogen, alkyl, cycloalkyl, alkylaminoalkyl, cycloalkylaminoalkyl, amino or haloalkyl;
R₆ is hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl or haloalkoxy, preferably hydrogen, deuterium, halogen, alkyl, cycloalkyl, cyano, amino, haloalkyl or haloalkoxy;
m, n and q are each independently 0, 1 or 2, and p is 1 or 2.

In other preferred embodiments of the present invention, in the compound of formula I-B described above:

### X is —CR₆═ or —N═;

each R₀ is independently hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C, preferably alkyl, cycloalkyl, methyl-d₃, ethyl-d₅, ethyl-2,2,2-d₃, isopropyl-d₇, methyl-¹⁴C, methyl-¹³C or methyl-¹¹C, and more preferably alkyl, methyl-d₃, ethyl-d₅, ethyl-2,2,2-d₃, isopropyl-d₇, methyl-¹⁴C, methyl-¹³C or methyl-¹¹C, wherein the alkyl is preferably C₁-C₆ alkyl, more preferably methyl, ethyl or isopropyl, and most preferably methyl;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl, wherein the alkyl is preferably C₁-C₆ alkyl, more preferably methyl, ethyl or isopropyl, and most preferably methyl; the cyanoalkyl is preferably C₂-C₆ cyanoalkyl, and more preferably cyanomethyl;
on the aromatic ring containing X, R₃, together with the tetrahydropyridopyrimidine ring, in the 1,8-disubstituted form is halogen, alkyl, methyl-d₃, ethyl-d₅, ethyl-2,2,2-d₃, isopropyl-d₇, methyl-¹⁴C, methyl-¹³C or methyl-¹¹C; the remaining R₃ is hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₃ is hydrogen, halogen, alkyl, methyl-d₃, ethyl-d₅, ethyl-2,2,2-d₃, isopropyl-d₇, methyl-¹⁴C, methyl-¹³C or methyl-¹¹C; more preferably, the remaining R₃ is hydrogen, halogen, alkyl, methyl-d₃, methyl-¹⁴C, methyl-¹³C or methyl-¹¹C, wherein the halogen is preferably fluorine, chlorine, bromine or iodine, more preferably fluorine, chlorine or bromine, and most preferably chlorine; the alkyl is preferably C₁-C₆ alkyl, more preferably methyl, ethyl or isopropyl, and most preferably methyl;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl, wherein halogen is preferably fluorine, chlorine, bromine or iodine, more preferably fluorine, chlorine or bromine, and most preferably chlorine; the cycloalkylaminoalkyl is preferably (C₃-C₆ cycloalkyl)-NH—(C₁₋₆ alkylene)-, and more preferably cyclopropylaminomethyl;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen; m, n and q are each independently 0, 1 or 2, and p is 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

In some embodiments of the present invention, the compound of formula I described above is a compound of formula I-C:

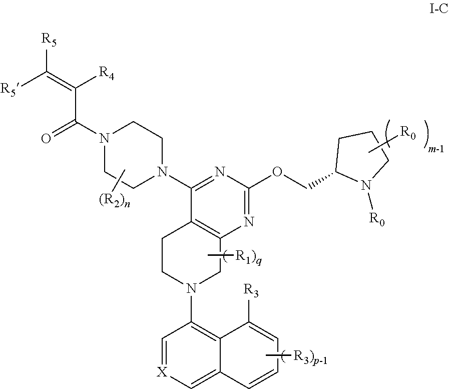

wherein X, R₀, R₁, R₂, R₃, R₄, R₅ or R_{5′} are as defined in formula I; m and p are each independently 1 or 2; n and q are each independently 0, 1 or 2.

In some preferred embodiments of the present invention, in the compound of formula I-C described above:

### X is —CR₆═ or —N═;

on the pyrrolidine ring, R₀ linked to the nitrogen atom is alkyl, methyl-d₃, ethyl-d₅, ethyl-2,2,2-d₃, isopropyl-d₇, methyl-¹⁴C, methyl-¹³C or methyl-¹¹C; the remaining R₀ is hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl ¹⁴C, methyl-¹³C or methyl-¹¹C; preferably, the remaining R₀ is hydrogen, alkyl, cycloalkyl, methyl-d₃, ethyl-d₅, ethyl-2,2,2-d₃, isopropyl-d₇, methyl ¹⁴C, methyl-¹³C or methyl-¹¹C; more preferably, the remaining R₀ is hydrogen, alkyl, methyl-d₃, ethyl-d₅, ethyl-2,2,2-d₃, isopropyl-d₇, methyl-¹⁴C, methyl-¹³C, or methyl-¹¹C, wherein the alkyl is preferably C₁-C₆ alkyl, more preferably methyl, ethyl, or isopropyl, and most preferably methyl;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl, wherein the alkyl is preferably C₁-C₆ alkyl, more preferably methyl, ethyl or isopropyl, and most preferably methyl; the cyanoalkyl is preferably C₂-C₆ cyanoalkyl, and more preferably cyanomethyl;
on the aromatic ring containing X, R₃, together with the tetrahydropyridopyrimidine ring, in the 1,8-disubstituted form is halogen, alkyl, methyl-d₃, ethyl-d₅, ethyl-2,2,2-d₃, isopropyl-d₇, methyl-¹⁴C, methyl ¹³C or methyl ¹¹C; the remaining R₃ is hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₃ is hydrogen, halogen, alkyl, methyl-d₃, ethyl-d₅, ethyl-2,2,2-d₃, isopropyl-d₇, methyl ¹⁴C, methyl-¹³C or methyl-¹¹C; more preferably, the remaining R₃ is hydrogen, halogen, alkyl, methyl-d₃, methyl-¹⁴C, methyl-¹³C or methyl-¹¹C, wherein the halogen is preferably fluorine, chlorine, bromine or iodine, more preferably fluorine, chlorine or bromine, and most preferably chlorine; the alkyl is preferably C₁-C₆ alkyl, more preferably methyl, ethyl or isopropyl, and most preferably methyl;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl, wherein halogen is preferably fluorine, chlorine, bromine or iodine, more preferably fluorine, chlorine or bromine, and most preferably chlorine; the cycloalkylaminoalkyl is preferably (C₃-C₆ cycloalkyl)-NH—(C₁₋₆ alkylene)-, and more preferably cyclopropylaminomethyl;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
m and p are each independently 1 or 2, and n and q are each independently 0, 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

In some more preferred embodiments of the present invention, in the compound of formula I-A, formula I-B or formula I-C described above:

### X is —CH═ or —N═;

each R₀ is independently alkyl, methyl-d₃, ethyl-d₅, ethyl-2,2,2-d₃, isopropyl-d₇, methyl-¹⁴C, methyl-¹³C, or methyl-¹¹C, wherein the alkyl is C₁-C₆ alkyl, preferably methyl, ethyl, or isopropyl, and more preferably methyl;
R₁ is hydrogen;
each R₂ is independently alkyl or cyanoalkyl, wherein the alkyl is C₁-C₆ alkyl, preferably methyl, ethyl or isopropyl, and more preferably methyl; the cyanoalkyl is —(C₁-C₆ alkylene)-CN, preferably cyanomethyl (—CH₂CN), 1-cyanoethyl (—CH(CN)CH₃) or 2-cyanoethyl (—CH₂CH₂CN), and more preferably cyanomethyl;
each R₃ is independently halogen, alkyl, methyl-d₃, methyl-¹⁴C, methyl-¹³C, or methyl-¹¹C, wherein the halogen is fluorine, chlorine, bromine, or iodine, preferably fluorine, chlorine, or bromine, and more preferably chlorine; the alkyl is C₁-C₆ alkyl, preferably methyl, ethyl or isopropyl, and more preferably methyl;
R₄, R₅ and R_{5′} are each independently hydrogen, halogen or cycloalkylaminoalkyl, wherein the halogen is fluorine, chlorine, bromine or iodine, preferably fluorine, chlorine or bromine, and more preferably fluorine; the cycloalkylaminoalkyl is —(C₁-C₄ alkylene)-NH—(C₃-C₆ cycloalkyl), preferably cyclopropylaminomethyl (c-PrNHCH₂—), cyclobutylaminomethyl (c-BuNHCH₂—), cyclopentylaminomethyl (c-PenNHCH₂—) or cyclohexylaminomethyl (c-HexNHCH₂—), and more preferably cyclopropylaminomethyl;
m, n, p and q are each independently 1 or 2, preferably 1;
and
at least one R₀ or R₃ is methyl-d₃, ethyl-d₅, ethyl-2,2,2-d₃, isopropyl-d₇, methyl-¹⁴C, methyl-¹³C, or methyl-¹¹C.

In addition, the present invention also provides the compounds of formula I (including the compounds of formula I-A, formula I-B and formula I-C), and the specific structures and the chemical names thereof are shown in the table below:

| No. | Structural formulas | Name |
|---|---|---|
| 1 | 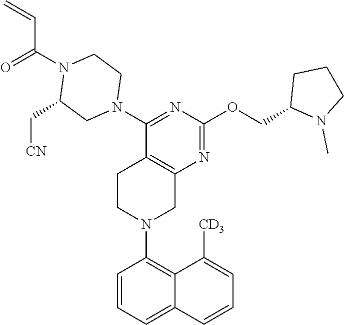 [SMILES: [2H]C([2H])([2H])c1cccc2cccc(N3CCc4c(nc(OC[C@@H]5CCCN5C)nc4N4CCN(C(=O)C=C)[C@@H](CC#N)C4)C3)c12] | 2-((S)-1-acryloyl-4-(7-(8-(methyl-d₃)naphthalen-1-yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 2 | 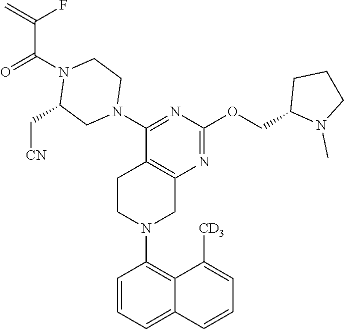 [SMILES: [2H]C([2H])([2H])c1cccc2cccc(N3CCc4c(nc(OC[C@@H]5CCCN5C)nc4N4CCN(C(=O)C(=C)F)[C@@H](CC#N)C4)C3)c12] | 2-((S)-1-(2-fluoroacryloyl)-4-(7-(8-(methyl-d₃)naphthalen-1- yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 3 | 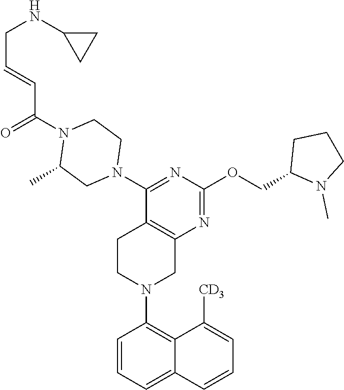 [SMILES: [2H]C([2H])([2H])c1cccc2cccc(N3CCc4c(nc(OC[C@@H]5CCCN5C)nc4N4CCN(C(=O)/C=C/CNC5CC5)[C@@H](C)C4)C3)c12] | (E)-4-(cyclopropylamino)-1-((S)-2-methyl-4-(7-(8-(methyl- d₃)naphthalen-1-yl)- 2-(((S)-1-methylpyrrolidin-2-yl)methoxy)-5,6,7,8- tetrahydropyrido[3,4-d]pyrimidin-4-yl)piperazin-1-yl)-2- buten-1-one |
| 4 | 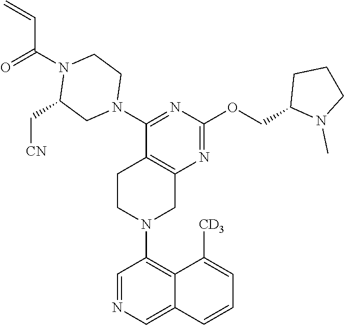 [SMILES: [2H]C([2H])([2H])c1cccc2cncc(N3CCc4c(nc(OC[C@@H]5CCCN5C)nc4N4CCN(C(=O)C=C)[C@@H](CC#N)C4)C3)c12] | 2-((S)-1-acryloyl-4-(7-(5-(methyl-d₃)isoquinolin-4-yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 5 | 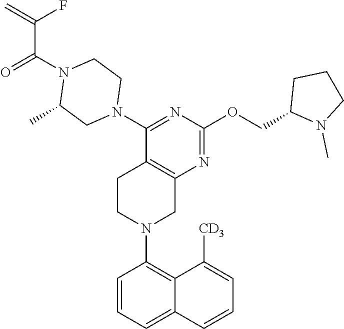 [SMILES: [2H]C([2H])([2H])c1cccc2cccc(N3CCc4c(nc(OC[C@@H]5CCCN5C)nc4N4CCN(C(=O)C(=C)F)[C@@H](C)C4)C3)c12] | 2-fluoro-1-((S)-2-methyl-4-(7-(8-(methyl-d₃)naphthalen-1-yl)- 2-(((S)-1-methylpyrrolidin-2-yl)methoxy)-5,6,7,8- tetrahydropyrido[3,4-d]pyrimidin-4-yl)piperazin-1-yl)-2- propen-1-one |
| 6 | 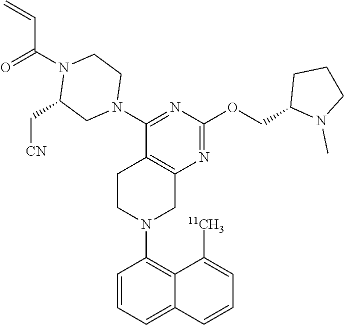 [SMILES: C=CC(=O)N1CCN(c2nc(OC[C@@H]3CCCN3C)nc3c2CCN(c2cccc4cccc([11CH3])c24)C3)C[C@@H]1CC#N] | 2-((S)-1-acryloyl-4-(7-(8-(methyl-¹¹C)naphthalen-1-yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 7 | 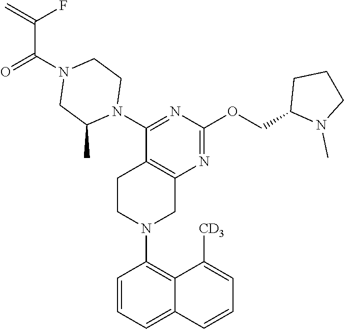 [SMILES: [2H]C([2H])([2H])c1cccc2cccc(N3CCc4c(nc(OC[C@@H]5CCCN5C)nc4N4CCN(C(=O)C(=C)F)C[C@@H]4C)C3)c12] | 2-fluoro-1-((S)-3-methyl-4-(7-(8-(methyl-d₃)naphthalen-1-yl)- 2-(((S)-1-methylpyrrolidin-2-yl)methoxy)-5,6,7,8- tetrahydropyrido[3,4-d]pyrimidin-4-yl)piperazin-1-yl)-2- propen-1-one |
| 8 | 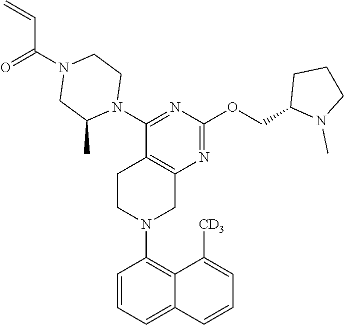 [SMILES: [2H]C([2H])([2H])c1cccc2cccc(N3CCc4c(nc(OC[C@@H]5CCCN5C)nc4N4CCN(C(=O)C=C)C[C@@H]4C)C3)c12] | 1-((S)-3-methyl-4-(7-(8-(methyl-d₃)naphthalen-1-yl)- 2-(((S)-1-methylpyrrolidin-2-yl)methoxy)-5,6,7,8- tetrahydropyrido[3,4-d]pyrimidin-4-yl)piperazin-1-yl)-2- propen-1-one |
| 9 | 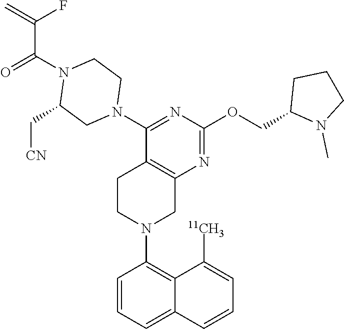 [SMILES: C=C(F)C(=O)N1CCN(c2nc(OC[C@@H]3CCCN3C)nc3c2CCN(c2cccc4cccc([11CH3])c24)C3)C[C@@H]1CC#N] | 2-((S)-1-(2-fluoroacryloyl)-4-(7-(8-(methyl-¹¹C)naphthalen-1- yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 10 | 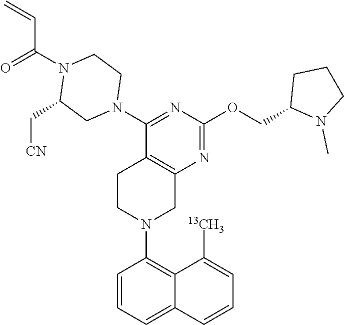 [SMILES: C=CC(=O)N1CCN(c2nc(OC[C@@H]3CCCN3C)nc3c2CCN(c2cccc4cccc([13CH3])c24)C3)C[C@@H]1CC#N] | 2-((S)-1-acryloyl-4-(7-(8-(methyl-¹³C)naphthalen-1-yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 11 | 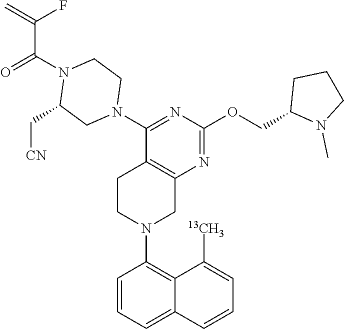 [SMILES: C=C(F)C(=O)N1CCN(c2nc(OC[C@@H]3CCCN3C)nc3c2CCN(c2cccc4cccc([13CH3])c24)C3)C[C@@H]1CC#N] | 2-((S)-1-(2-fluoroacryloyl)-4-(7-(8-(methyl-¹³C)naphthalen-1- yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 12 | 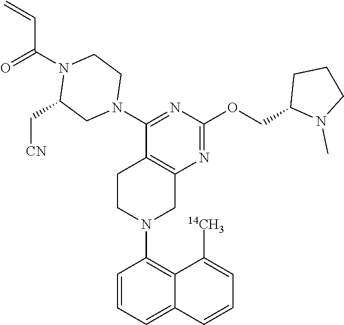 [SMILES: C=CC(=O)N1CCN(c2nc(OC[C@@H]3CCCN3C)nc3c2CCN(c2cccc4cccc([14CH3])c24)C3)C[C@@H]1CC#N] | 2-((S)-1-acryloyl-4-(7-(8-(methyl-¹⁴C)naphthalen-1-yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 13 | 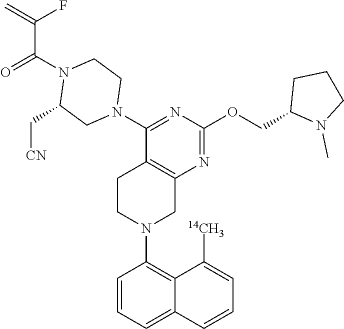 [SMILES: C=C(F)C(=O)N1CCN(c2nc(OC[C@@H]3CCCN3C)nc3c2CCN(c2cccc4cccc([14CH3])c24)C3)C[C@@H]1CC#N] | 2-((S)-1-(2-fluoroacryloyl)-4-(7-(8-(methyl-¹⁴C)naphthalen-1- yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 14 | 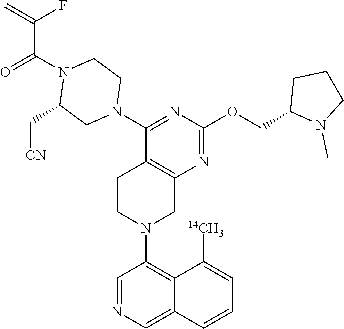 [SMILES: C=C(F)C(=O)N1CCN(c2nc(OC[C@@H]3CCCN3C)nc3c2CCN(c2cncc4cccc([14CH3])c24)C3)C[C@@H]1CC#N] | 2-((S)-1-(2-fluoroacryloyl)-4-(7-(5-(methyl-¹⁴C)isoquinolin-4- yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 15 | 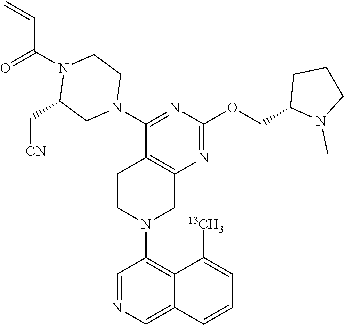 [SMILES: C=CC(=O)N1CCN(c2nc(OC[C@@H]3CCCN3C)nc3c2CCN(c2cncc4cccc([13CH3])c24)C3)C[C@@H]1CC#N] | 2-((S)-1-acryloyl-4-(7-(5-(methyl-¹³C)isoquinolin-4-yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 16 | 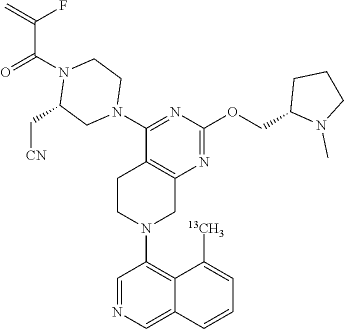 [SMILES: C=C(F)C(=O)N1CCN(c2nc(OC[C@@H]3CCCN3C)nc3c2CCN(c2cncc4cccc([13CH3])c24)C3)C[C@@H]1CC#N] | 2-((S)-1-(2-fluoroacryloyl)-4-(7-(5-(methyl-¹³C)isoquinolin-4- yl)- 2-(((S)-1-methylpyrrolidin-2-yl)methoxy)-5,6,7,8- tetrahydropyrido[3,4-d]pyrimidin-4-yl)piperazin-2- yl)acetonitrile |
| 17 | 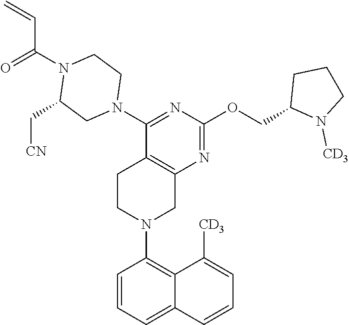 [SMILES: [2H]C([2H])([2H])c1cccc2cccc(N3CCc4c(nc(OC[C@@H]5CCCN5C([2H])([2H])[2H])nc4N4CCN(C(=O)C=C)[C@@H](CC#N)C4)C3)c12] | 2-((S)-1-acryloyl-4-(7-(8-(methyl-d₃)naphthalen-1-yl)- 2-(((S)-1-(methyl-d₃)pyrrolidin-2-yl)methoxy)- 5,6,7,8-tetrahydropyrido[3,4-d]pyrimidin-4-yl) piperazin-2-yl)acetonitrile |
| 18 | 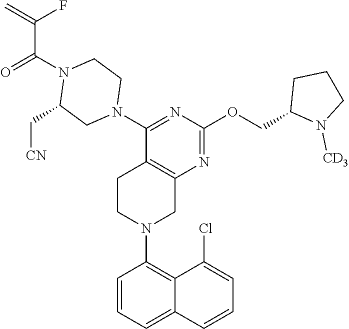 [SMILES: [2H]C([2H])([2H])N1CCC[C@H]1COc1nc2c(c(N3CCN(C(=O)C(=C)F)[C@@H](CC#N)C3)n1)CCN(c1cccc3cccc(Cl)c13)C2] | 2-((S)-4-(7-(8-chloronaphthalen-1-yl)-2-(((S)-1-(methyl-d₃) pyrrolidin-2-yl)methoxy)-5,6,7,8-tetrahydropyrido [3,4-d]pyrimidin-4-yl)-1-(2-fluoroacryloyl) piperazin-2-yl)acetonitrile |
| 19 | 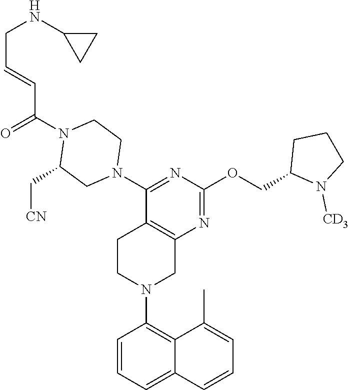 [SMILES: [2H]C([2H])([2H])N1CCC[C@H]1COc1nc2c(c(N3CCN(C(=O)/C=C/CNC4CC4)[C@@H](CC#N)C3)n1)CCN(c1cccc3cccc(C)c13)C2] | 2-((S)-1-((E)-4-(cyclopropylamino)-2-butenoyl)-4- (2-(((S)-1-(methyl-d₃)pyrrolidin-2-yl)methoxy)- 7-(8-methylnaphthalen-1-yl)-5,6,7,8-tetrahydropyrido[3,4-d] pyrimidin-4-yl)piperazin-2-yl)acetonitrile |
| 20 | 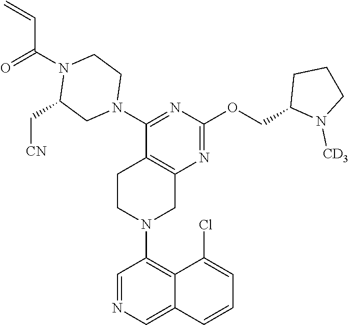 [SMILES: [2H]C([2H])([2H])N1CCC[C@H]1COc1nc2c(c(N3CCN(C(=O)C=C)[C@@H](CC#N)C3)n1)CCN(c1cncc3cccc(Cl)c13)C2] | 2-((S)-1-acryloyl-4-(7-(5-chloroisoquinolin-4-yl)- 2-(((S)-1-(methyl-d₃)pyrrolidin-2-yl)methoxy)- 5,6,7,8-tetrahydropyrido[3,4-d]pyrimidin-4-yl) piperazin-2-yl)acetonitrile |
| 21 | 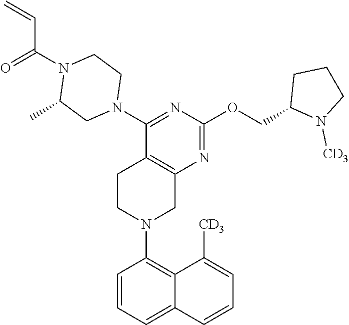 [SMILES: [2H]C([2H])([2H])c1cccc2cccc(N3CCc4c(nc(OC[C@@H]5CCCN5C([2H])([2H])[2H])nc4N4CCN(C(=O)C=C)[C@@H](C)C4)C3)c12] | 1-((S)-2-methyl-4-(7-(8-(methyl-d₃)naphthalen-1-yl)- 2-(((S)-1-(methyl-d₃)pyrrolidin-2-yl)methoxy)- 5,6,7,8-tetrahydropyrido[3,4-d]pyrimidin-4-yl) piperazin-1-yl)-2-propen-1-one |
| 22 | 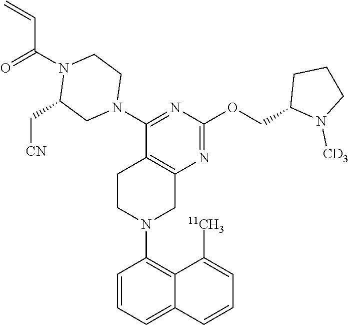 [SMILES: [2H]C([2H])([2H])N1CCC[C@H]1COc1nc2c(c(N3CCN(C(=O)C=C)[C@@H](CC#N)C3)n1)CCN(c1cccc3cccc([11CH3])c13)C2] | 2-((S)-1-acryloyl-4-(2-(((S)-1-(methyl-d₃) pyrrolidin-2-yl)methoxy)-7-(8-(methyl-¹¹C)naphthalen-1-yl)- 5,6,7,8-tetrahydropyrido[3,4-d]pyrimidin-4-yl) piperazin-2-yl)acetonitrile |
| 23 | 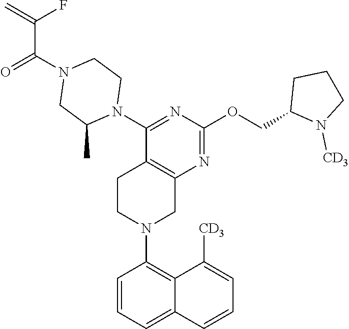 [SMILES: [2H]C([2H])([2H])c1cccc2cccc(N3CCc4c(nc(OC[C@@H]5CCCN5C([2H])([2H])[2H])nc4N4CCN(C(=O)C(=C)F)C[C@@H]4C)C3)c12] | 2-fluoro-1-((S)-3-methyl-4-(7-(8-(methyl-d₃)naphthalen-1-yl)- 2-(((S)-1-(methyl-d₃)pyrrolidin-2-yl)methoxy)- 5,6,7,8-tetrahydropyrido[3,4-d]pyrimidin-4-yl) piperazin-1-yl)-2-propen-1-one |
| 24 | 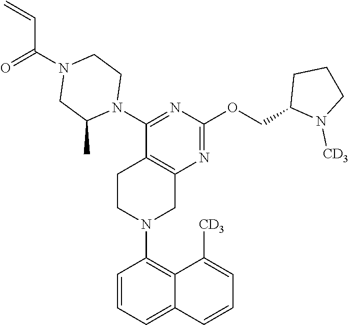 [SMILES: [2H]C([2H])([2H])c1cccc2cccc(N3CCc4c(nc(OC[C@@H]5CCCN5C([2H])([2H])[2H])nc4N4CCN(C(=O)C=C)C[C@@H]4C)C3)c12] | 1-((S)-3-methyl-4-(7-(8-(methyl-d₃)naphthalen-1-yl)- 2-(((S)-1-(methyl-d₃)pyrrolidin-2-yl)methoxy)- 5,6,7,8-tetrahydropyrido[3,4-d]pyrimidin-4-yl) piperazin-1-yl)-2-propen-1-one |
| 25 | 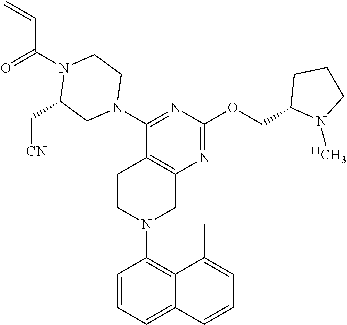 [SMILES: C=CC(=O)N1CCN(c2nc(OC[C@@H]3CCCN3[11CH3])nc3c2CCN(c2cccc4cccc(C)c24)C3)C[C@@H]1CC#N] | 2-((S)-1-acryloyl-4-(2-(((S)-1-(methyl-¹¹C) pyrrolidin-2-yl)methoxy)-7-(8-methylnaphthalen-1-yl)- 5,6,7,8-tetrahydropyrido[3,4-d]pyrimidin-4-yl) piperazin-2-yl)acetonitrile |
| 26 | 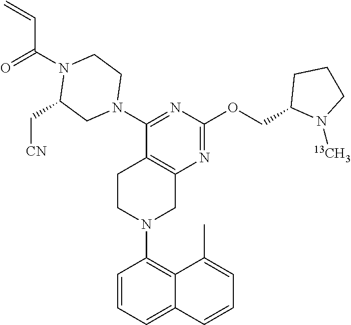 [SMILES: C=CC(=O)N1CCN(c2nc(OC[C@@H]3CCCN3[13CH3])nc3c2CCN(c2cccc4cccc(C)c24)C3)C[C@@H]1CC#N] | 2-((S)-1-acryloyl-4-(2-(((S)-1-(methyl-¹³C) pyrrolidin-2-yl)methoxy)-7-(8-methylnaphthalen-1-yl)- 5,6,7,8-tetrahydropyrido[3,4-d]pyrimidin-4-yl) piperazin-2-yl)acetonitrile |
| 27 | 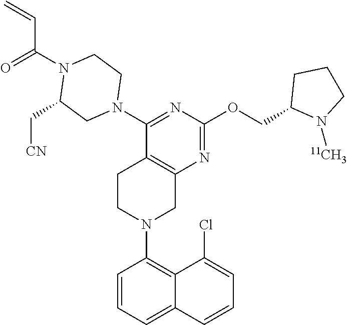 [SMILES: C=CC(=O)N1CCN(c2nc(OC[C@@H]3CCCN3[11CH3])nc3c2CCN(c2cccc4cccc(Cl)c24)C3)C[C@@H]1CC#N] | 2-((S)-1-acryloyl-4-(7-(8-chloronaphthalene-1-yl)- 2-(((S)-1-(methyl-¹¹C)pyrrolidin-2-yl)methoxy)- 5,6,7,8-tetrahydropyrido[3,4-d]pyrimidin-4-yl) piperazin-2-yl)acetonitrile |
| 28 | 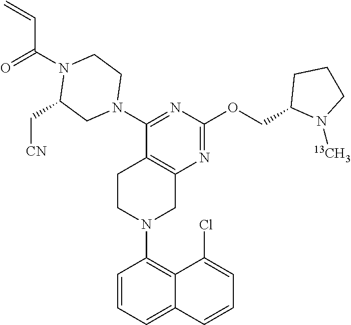 [SMILES: C=CC(=O)N1CCN(c2nc(OC[C@@H]3CCCN3[13CH3])nc3c2CCN(c2cccc4cccc(Cl)c24)C3)C[C@@H]1CC#N] | 2-((S)-1-acryloyl-4-(7-(8-chloronaphthalene-1-yl)- 2-(((S)-1-(methyl-¹³C)pyrrolidin-2-yl)methoxy)- 5,6,7,8-tetrahydropyrido[3,4-d]pyrimidin-4-yl) piperazin-2-yl)acetonitrile |
| 29 | 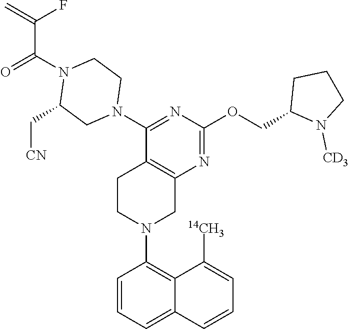 [SMILES: [2H]C([2H])([2H])N1CCC[C@H]1COc1nc2c(c(N3CCN(C(=O)C(=C)F)[C@@H](CC#N)C3)n1)CCN(c1cccc3cccc([14CH3])c13)C2] | 2-((S)-1-(2-fluoroacryloyl)-4-(2-(((S)-1-(methyl-d₃) pyrrolidin-2-yl)methoxy)-7-(8-(methyl-¹⁴C)naphthalen-1-yl)- 5,6,7,8-tetrahydropyrido[3,4-d]pyrimidin-4-yl) piperazin-2-yl)acetonitrile |
| 30 | 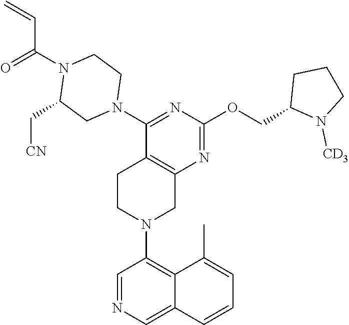 [SMILES: [2H]C([2H])([2H])N1CCC[C@H]1COc1nc2c(c(N3CCN(C(=O)C=C)[C@@H](CC#N)C3)n1)CCN(c1cncc3cccc(C)c13)C2] | 2-((S)-1-acryloyl-4-(2-(((S)-1-(methyl-d₃) pyrrolidin-2-yl)methoxy)-7-(5-methylisoquinolin-4-yl)- 5,6,7,8-tetrahydropyrido[3,4-d]pyrimidin-4-yl) piperazin-2-yl)acetonitrile |
| 31 | 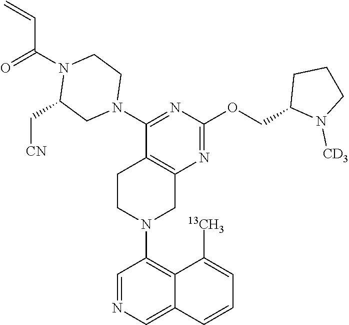 [SMILES: [2H]C([2H])([2H])N1CCC[C@H]1COc1nc2c(c(N3CCN(C(=O)C=C)[C@@H](CC#N)C3)n1)CCN(c1cncc3cccc([13CH3])c13)C2] | 2-((S)-1-acryloyl-4-(2-(((S)-1-(methyl-d₃)pyrrolidine- 2-yl)methoxy)-7-(5-(methyl-¹³C)isoquinolin-4-yl)- 5,6,7,8-tetrahydropyrido[3,4-d]pyrimidin-4-yl) piperazin-2-yl)acetonitrile |
| 32 | 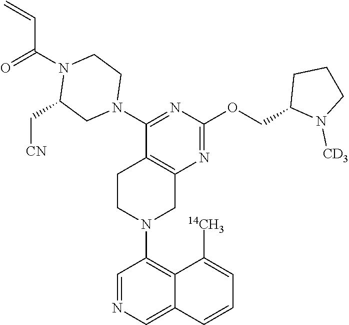 [SMILES: [2H]C([2H])([2H])N1CCC[C@H]1COc1nc2c(c(N3CCN(C(=O)C=C)[C@@H](CC#N)C3)n1)CCN(c1cncc3cccc([14CH3])c13)C2] | 2-((S)-1-acryloyl-4-(2-(((S)-1-(methyl-d₃)pyrrolidine- 2-yl)methoxy)-7-(5-(methyl-¹⁴C)isoquinolin-4-yl)- 5,6,7,8-tetrahydropyrido[3,4-d]pyrimidin-4-yl) piperazin-2-yl)acetonitrile |
| 33 | 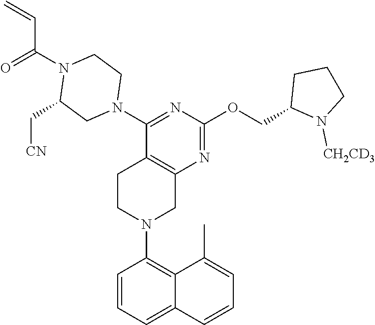 | 2-((S)-1-acryloyl-4-(2-(((S)-1-(ethyl-2,2,2-d₃) pyrrolidin-2-yl)methoxy)-7-(8-methylnaphthalen-1-yl)- 5,6,7,8-tetrahydropyrido[3,4-d]pyrimidin-4-yl) piperazin-2-yl)acetonitrile |
| 34 | 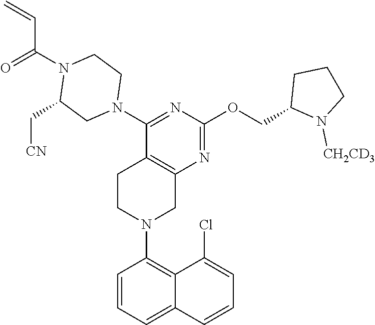 | 2-((S)-1-acryloyl-4-(7-(8-chloronaphthalene-1-yl)- 2-(((S)-1-(ethyl-2,2,2-d₃)pyrrolidin-2-yl)methoxy)- 5,6,7,8-tetrahydropyrido[3,4-d]pyrimidin-4-yl) piperazin-2-yl)acetonitrile |
| 35 | 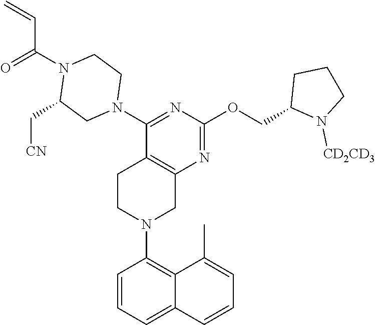 | 2-((S)-1-acryloyl-4-(2-(((S)-1-(ethyl-d₅) pyrrolidin-2-yl)methoxy)-7-(8-methylnaphthalen-1-yl)- 5,6,7,8-tetrahydropyrido[3,4-d]pyrimidin-4-yl) piperazin-2-yl)acetonitrile |
| 36 | 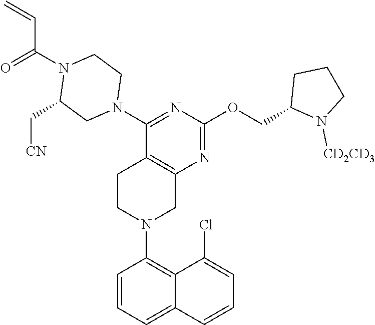 | 2-((S)-1-acryloyl-4-(7-(8-chloronaphthalene-1-yl)- 2-(((S)-1-(ethyl-d₅)pyrrolidin-2-yl)methoxy)- 5,6,7,8-tetrahydropyrido[3,4-d]pyrimidin-4-yl) piperazin-2-yl)acetonitrile |
| 37 | 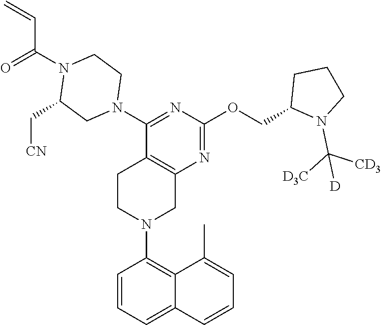 [SMILES: [2H]C([2H])([2H])C([2H])(N1CCC[C@H]1COc1nc2c(c(N3CCN(C(=O)C=C)[C@@H](CC#N)C3)n1)CCN(c1cccc3cccc(C)c13)C2)C([2H])([2H])[2H]] | 2-((S)-1-acryloyl-4-(7-(8-methylnaphthalen-1-yl)- 2-(((S)-1-(isopropyl-d₇)pyrrolidin-2-yl)methoxy)- 5,6,7,8-tetrahydropyrido[3,4-d]pyrimidin-4-yl) piperazin-2-yl)acetonitrile |
| 38 | 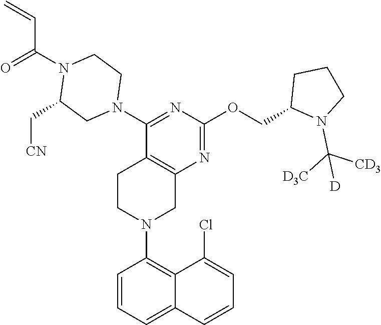 [SMILES: [2H]C([2H])([2H])C([2H])(N1CCC[C@H]1COc1nc2c(c(N3CCN(C(=O)C=C)[C@@H](CC#N)C3)n1)CCN(c1cccc3cccc(Cl)c13)C2)C([2H])([2H])[2H]] | 2-((S)-1-acryloyl-4-(7-(8-chloronaphthalene-1-yl)- 2-(((S)-1-(isopropyl-d₇)pyrrolidin-2-yl)methoxy)- 5,6,7,8-tetrahydropyrido[3,4-d]pyrimidin-4-yl) piperazin-2-yl)acetonitrile |
| 39 | 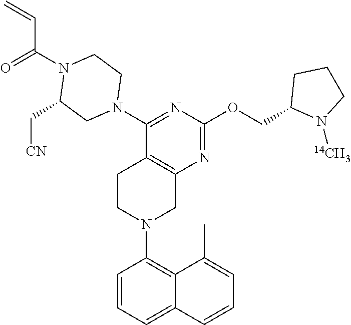 [SMILES: C=CC(=O)N1CCN(c2nc(OC[C@@H]3CCCN3[14CH3])nc3c2CCN(c2cccc4cccc(C)c24)C3)C[C@@H]1CC#N] | 2-((S)-1-acryloyl-4-(2-(((S)-1-(methyl-¹⁴C) pyrrolidin-2-yl)methoxy)-7-(8-methylnaphthalen-1-yl)- 5,6,7,8-tetrahydropyrido[3,4-d]pyrimidin-4-yl) piperazin-2-yl)acetonitrile |
| 40 | 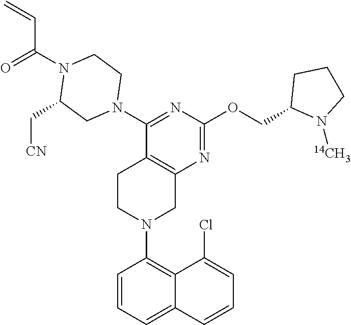 [SMILES: C=CC(=O)N1CCN(c2nc(OC[C@@H]3CCCN3[14CH3])nc3c2CCN(c2cccc4cccc(Cl)c24)C3)C[C@@H]1CC#N] | 2-((S)-1-acryloyl-4-(7-(8-chloronaphthalene-1-yl)- 2-(((S)-1-(methyl-¹⁴C)pyrrolidin-2-yl)methoxy)- 5,6,7,8-tetrahydropyrido[3,4-d]pyrimidin-4-yl) piperazin-2-yl)acetonitrile |
| 41 | 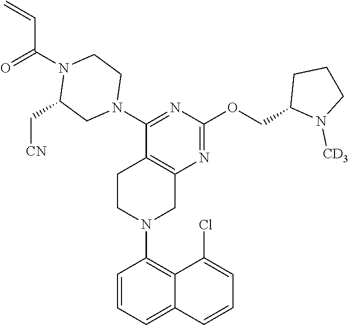 [SMILES: [2H]C([2H])([2H])N1CCC[C@H]1COc1nc2c(c(N3CCN(C(=O)C=C)[C@@H](CC#N)C3)n1)CCN(c1cccc3cccc(Cl)c13)C2] | 2-((S)-1-acryloyl-4-(7-(8-chloronaphthalene-1-yl)- 2-(((S)-1-(methyl-d₃)pyrrolidin-2-yl)methoxy)- 5,6,7,8-tetrahydropyrido[3,4-d]pyrimidin-4-yl) piperazin-2-yl)acetonitrile |
| 42 | 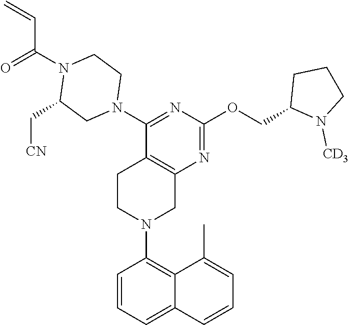 [SMILES: [2H]C([2H])([2H])N1CCC[C@H]1COc1nc2c(c(N3CCN(C(=O)C=C)[C@@H](CC#N)C3)n1)CCN(c1cccc3cccc(C)c13)C2] | 2-((S)-1-acryloyl-4-(7-(8-methylnaphthalen-1-yl)- 2-(((S)-1-(methyl-d₃)pyrrolidin-2-yl)methoxy)- 5,6,7,8-tetrahydropyrido[3,4-d]pyrimidin-4-yl) piperazin-2-yl)acetonitrile |

### [Preparation Method]

The present invention provides a method for preparing the compound of formula I described above comprising:

1) reacting compound I-1 with compound I-2 to obtain compound I-3;

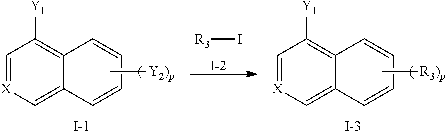

2) reacting compound I-3 with compound I-4 to obtain compound I-5;

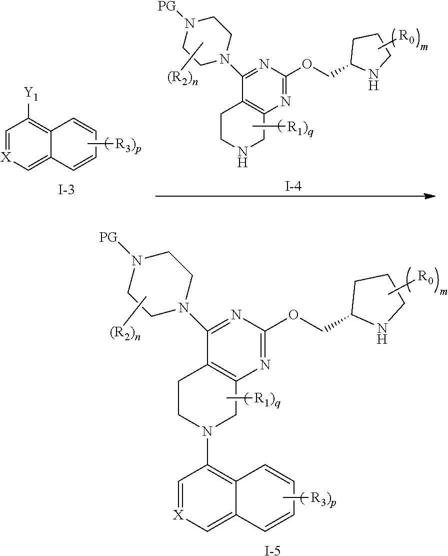

3) subjecting compound I-5 to deprotection reaction to obtain compound I-6; and

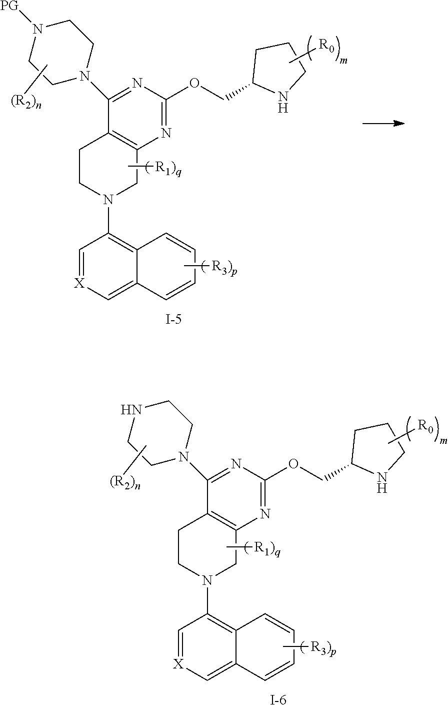

4) reacting compound I-6 with compound I-7 to obtain the compound of formula I;

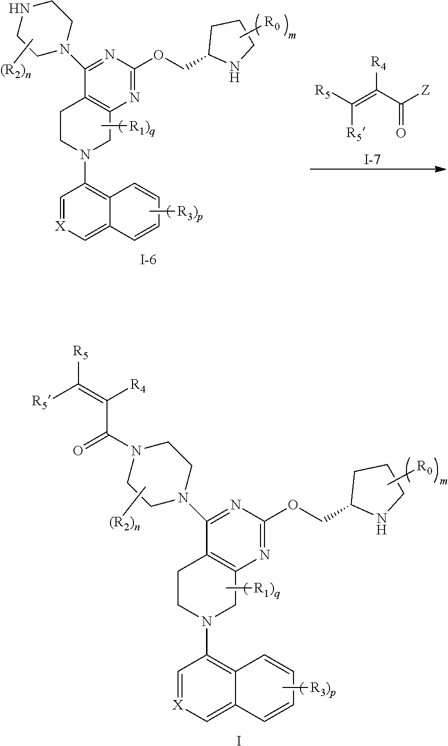

wherein Y₁ and Y₂ are each independently chlorine, bromine, iodine, methanesulfonyloxy, trifluoromethanesulfonyloxy, p-toluenesulfonyloxy, a borate ester, a zinc halide group, a magnesium halide group, or a tin halide group; z is hydroxy, bromine or chlorine; PG represents a protecting group; x, R₀, R₁, R₂, R₃, R₄, R₅, R_{5′}, m, n, p and q are as defined in formula I.

In some embodiments of the present invention, step 1) and/or step 2) of the preparation method described above is performed through a substitution reaction under a basic condition. The basic reagent employed includes, but is not limited to, triethylamine (TEA), sodium hydride (NaH), potassium tert-butoxide, sodium carbonate, potassium carbonate, sodium bicarbonate, potassium bicarbonate, N,N-diisopropylethylamine (DIPEA), pyridine, triethylene diamine (TEDA), 1,8-diazabicyclo[5.4.0]undec-7-ene (DBU), 4-dimethylaminopyridine (DMAP), N-methylmorpholine, tetramethylethylenediamine, potassium hexamethyldisilazide, sodium hexamethyldisilazide, etc.

In some embodiments of the present invention, step 1) and/or step 2) of the preparation method described above is performed through a coupling reaction. The coupling reaction includes, but is not limited to, Buchwald-Hartwig Reaction, Suzuki Reaction, Heck Reaction, Stille Reaction, Sogonoshira Coupling, Kumada Coupling, Negishi Coupling, Hiyama Coupling, etc. The basic reagent employed includes, but is not limited to, sodium carbonate, potassium carbonate, cesium carbonate, etc. The catalyst employed includes, but is not limited to, Pd₂(dba)₃, Pd(PPh₃)₄, Pd(dppf)₂Cl₂, etc.

In some embodiments of the present invention, the protecting group and deprotection condition in step 3) of the preparation method described above includes, but is not limited to, the combinations shown in the table below:

| Protecting groups | Abbreviations | Deprotection conditions |
|---|---|---|
| 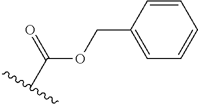 [SMILES: *C(=O)OCc1ccccc1] | Cbz | H₂/Pd-C, hydrogen donor/Pd-C, BBr₃/CH₂Cl₂, trifluoroacetic acid, HBr/HOAc, ACE-Cl/dichloroethane, etc. |
| 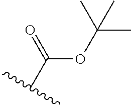 [SMILES: *C(=O)OC(C)(C)C] | Boc | HCl solution, trifluoroacetic acid, HBr, p-toluenesulfonic acid, etc. |
| 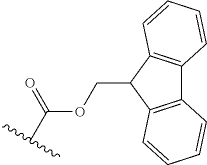 [SMILES: *C(=O)OCC1c2ccccc2-c2ccccc21] | Fmoc | piperidine/DMF/H₂O, ethylenediamine/DMF, HBr/AcOH, Na/liquid nitrogen, etc. |
| 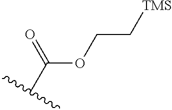 [SMILES: *C(=O)OCC[Si](C)(C)C] | Teoc | TBAF, TEAF, etc. |
| 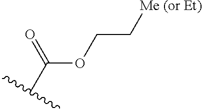 | — | HBr, Me₃SiI, KOH/H₂O/ethylene glycol, etc. |
| 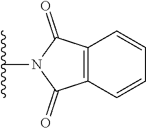 [SMILES: *N1C(=O)c2ccccc2C1=O] | Pht | NH₂NH₂/EtOH, NaBH₄/i-PrOH-H₂O, etc. |
| 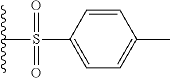 [SMILES: *S(=O)(=O)c1ccc(C)cc1] | Tos | HBr/HOAc, 48% HBr/phenol, etc. |
| 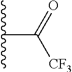 [SMILES: *C(=O)C(F)(F)F] | Tfa | K₂CO₃/MeOH/H₂O, NH₃/MeOH, HCl/MeOH, etc. |
| 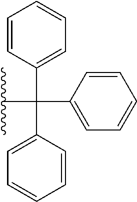 [SMILES: *C(c1ccccc1)(c1ccccc1)c1ccccc1] | Trt | HCl/MeOH, H₂/Pd, TFA/CH₂Cl₂, etc. |
| 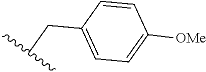 [SMILES: *Cc1ccc(OC)cc1] | PMB | HCO₂H/Pd-C/MeOH, H₂/Pd(OH)₂/EtOH, TFA, CAN/CH₃CN, etc. |
| 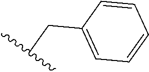 [SMILES: *Cc1ccccc1] | Bn | HCO₂H/Pd-C/MeOH, H₂/Pd(OH)₂/EtOH, CCl₃CH₂OCOCl/CH₃CN, etc. |

In some embodiments of the present invention, step 4) of the preparation method described above is performed through a substitution reaction under a basic condition. The basic reagent employed includes, but is not limited to, triethylamine, sodium hydride, potassium tert-butoxide, sodium carbonate, potassium carbonate, sodium bicarbonate, potassium bicarbonate, N,N-diisopropylethylamine, pyridine, triethylene diamine, 1,8-diazabicyclo[5.4.0]undec-7-ene, 4-dimethylaminopyridine, N-methylmorpholine, tetramethylethylenediamine, potassium hexamethyldisilazide, sodium hexamethyldisilazide, etc.

In some embodiments of the present invention, step 4) of the preparation method described above is performed through a condensation reaction. The condensing reagent employed includes, but is not limited to, N,N′-dicyclohexylcarbodiimide (DCC), N,N′-diisopropylcarbodiimide (DIC), N-(3-dimethylaminopropyl)-N-ethylcarbodiimide hydrochloride (EDC HCl), 4,5-dicyanoimidazole (DCI), N,N′-carbonyldiimidazole (CDI), N-hydroxysuccinimide (HOSu), N-hydroxythiosuccinimide sodium salt, Castro's condensing reagent (BOP), benzotriazol-1-yl-oxytripyrrolidinylphosphine (PyBOP), tripyrrolidinylphosphonium bromide hexafluorophosphate (PyBrOP), 1-hydroxy-7-azobenzotriazol (HOAT), 1-hydroxybenzotriazole (HOBt), 6-chloro-1-hydroxybenzotriazole (Cl—HOBt), O-(7-azabenzotriazole)-N,N,N′,N′-tetramethyluronium hexafluorophosphate (HATU), benzotriazol-N,N,N′,N′-tetramethyluronium Hexafluorophosphate (HBTU), O-benzotriazol-N,N,N′,N′-tetramethyluronium tetrafluoroborate (TBTU), 6-chlorobenzotriazole-1,1,3,3-tetramethyluronium hexafluorophosphate (HCTU), O-(1,2-dihydro-2-oxo-pyridyl)-1,1,3,3-tetramethyluronium tetrafluoroborate (TPTU), etc.

When the compound of the formula I described above has a specific configuration, the present invention also provides a corresponding preparation method so as to obtain the compound with the specific configuration. These compounds with specific configurations and the preparation methods therefor are likewise part of the present invention.

### [Composition]

The term “pharmaceutical composition” refers to a composition that can be used as a medicament. It comprises an active pharmaceutical ingredient (API) and optionally one or more pharmaceutically acceptable carriers.

The term “contrast agent composition” refers to a composition that can be used as a contrast agent. It comprises an imaging agent/contrast agent/contrast medium and optionally one or more pharmaceutically acceptable carriers.

The term “tracer composition” refers to a composition that can be used as a tracer. It comprises a tracing agent/tracer and optionally one or more pharmaceutically acceptable carriers.

The term “pharmaceutically acceptable carrier” refers to a pharmaceutical excipient that is compatible with the active pharmaceutical ingredient and is not deleterious to a subject. It includes, but is not limited to, diluents (or fillers), binders, disintegrants, lubricants, wetting agents, thickening agents, glidants, flavoring agents, smell correctives, preservatives, antioxidants, pH adjusters, solvents, co-solvents, surfactants, etc.

The present invention provides a pharmaceutical composition comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above.

The present invention provides a contrast agent composition comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above.

The present invention provides a tracer composition comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above.

In some preferred embodiments of the present invention, the pharmaceutical composition, contrast agent composition and/or tracer composition described above further comprises a pharmaceutically acceptable carrier.

### [Medical Use]

The compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, as well as the pharmaceutical composition, the contrast agent or the tracer composition described above, is capable of inhibiting KRAS G12C protein and further the phosphorylation of the downstream signal (p-ERK), and therefore can be used as a KRAS G12C protein inhibitor. Accordingly, the present invention provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition, the contrast agent or the tracer composition described above, as a KRAS G12C protein inhibitor.

In addition, the present application provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition described above, in preparing a medicament for preventing and/or treating a disease at least partially mediated by KRAS G12C protein.

In addition, the present application provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the contrast agent composition described above, in preparing a contrast agent kit for diagnosing a disease at least partially mediated by KRAS G12C protein.

In addition, the present application provides use of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the tracer composition described above, in preparing a tracer kit for diagnosing a disease at least partially mediated by KRAS G12C protein. The term “a disease at least partially mediated by KRAS G12C protein” refers to a disease whose pathogenesis includes at least a portion of the factors associated with KRAS G12C protein. Such diseases include, but are not limited to, cancer (e.g., cervical cancer), proliferative diseases, inflammation, ocular diseases (e.g., cataracts), autoimmune diseases (e.g., rheumatoid arthritis), etc.

### [Treatment Method]

The present invention provides a method for preventing and/or treating a disease at least partially mediated by KRAS G12C protein comprising: administering to an individual in need thereof a therapeutically effective amount of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion, or the pharmaceutical composition described above.

The term “therapeutically effective amount” refers to an amount of an active pharmaceutical ingredient that can induce a biological or medical response in a cell, tissue, organ or organism (e.g., an individual).

The term “administer/administration/administering” refers to the process of applying an active pharmaceutical ingredient (e.g., the compound of the present invention) or a pharmaceutical composition comprising an active pharmaceutical ingredient (e.g., the pharmaceutical composition of the present invention) to an individual or a site such as cell, tissue, organ, biological fluid, etc. thereof, such that the active pharmaceutical ingredient or pharmaceutical composition contacts the individual or the site such as cell, tissue, organ, biological fluid, etc. thereof. Common modes of administration include, but are not limited to, oral administration, subcutaneous administration, intramuscular administration, intraperitoneal administration, ocular administration, nasal administration, sublingual administration, rectal administration, vaginal administration, etc.

The term “in need thereof” refers to a judgment by a physician or other caregivers that an individual needs or will obtain benefits from a prophylactic and/or therapeutic procedure, the judgment being made based on various factors of the physician or other caregivers in their area of expertise.

The term “individual” (or subject) refers to a human or non-human animal (e.g., a mammal).

### [Combination Administration]

The present invention provides a pharmaceutical combination form comprising the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition described above, and at least one additional therapeutic agent for cancer.

The term “cancer” refers to a cellular disorder characterized by uncontrolled or unregulated cell proliferation, reduced cell differentiation, an inappropriate ability to invade surrounding tissues, and/or an ability to start new growth at different sites. Common cancers include, but are not limited to, brain cancer, liver cancer, gall bladder cancer, bronchial cancer, lung cancer, bladder cancer, ovarian cancer, cervical cancer, testicular cancer, lip cancer, tongue cancer, hypopharynx cancer, larynx cancer, esophageal cancer, gastric cancer, intestinal cancer (e.g., colon cancer, rectal cancer), thyroid cancer, salivary gland cancer, pancreatic cancer, breast cancer, prostate cancer, blood cancer (or leukemia), lymph cancer (or lymphoma), osteocarcinoma, and skin cancer.

The term “therapeutic agent for cancer” refers to a pharmaceutical composition or pharmaceutical formulation effective in controlling and/or combating cancer, including, but not limited to, cytotoxic drugs, anti-angiogenic drugs, DNA repair agents, epigenetic disruptors, immunomodulators, etc. Common therapeutic agents for cancer include, but are not limited to, anti-purines (e.g., pentostatin), anti-pyrimidines (e.g., fluorouracil), antifolates (e.g., methotrexate), DNA polymerase inhibitors (e.g., cytarabine), alkylating agents (e.g., cyclophosphamide), platinum-based complexes (e.g., cisplatin), DNA damaging antibiotics (e.g., mitomycin), topoisomerase inhibitors (e.g., camptothecin), drugs for intercalating DNA to interfere with synthesis of nucleic acids (e.g., epirubicin), drugs for disrupting ingredient supply (e.g., asparaginase), drugs for interfering with tubulin formation (e.g., paclitaxel), drugs for interfering with ribosome functions (e.g., harringtonine), cytokines (e.g., IL-1), thymopeptides, tumor cell proliferation viruses (e.g., adenovirus ONYX-015), DNA repair agents such as PARP inhibitors (e.g., Olaparib, Talazoparib, and Niraparib), anti-angiogenic drugs such as HIF-1 inhibitors (e.g., Roxadustat/FG-4592, 2-methoxy estradiol/2-MeOE2, and FG-2216) or VEGF signaling pathway inhibitors (e.g., bevacizumab, sunitinib, and sorafenib), epigenetic disruptors (e.g., HADC inhibitors), histone demethylation inhibitors, immune checkpoint inhibitors (e.g., PD-1/PD-L1 monoclonal antibody, and CTLA-4 monoclonal antibody), IDO inhibitors, etc.

In addition, the present invention provides a method for preventing and/or treating cancer, the method comprising: administering to an individual in need thereof a therapeutically effective amount of the compound of formula I (including compounds of formula I-A, formula I-B and formula I-C) or the pharmaceutically acceptable salt, the solvate, the hydrate, the stereoisomer, the tautomer, the isotopically labeled compound or the prodrug thereof or the mixture thereof in any proportion described above, or the pharmaceutical composition described above, or the pharmaceutical combination form described above.

The present invention will be further illustrated below with reference to specific examples. It should be understood that these examples are merely intended to illustrate the present invention rather than limit the scope of the present invention. If specific conditions are not indicated in the experimental procedures in the following examples, the conventional conditions or the conditions recommended by the manufacturers are usually followed. Unless otherwise indicated, percentages and parts appearing in the following examples are by weight.

### Intermediate Preparation Example 1: Synthesis of 1-bromo-8-(methyl-d₃)naphthalene (Intermediate A)

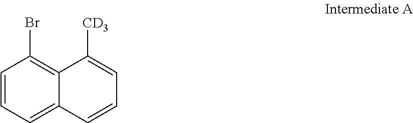 [SMILES: [2H]C([2H])([2H])c1cccc2cccc(Br)c12]

### Step 1: Synthesis of 8-bromonaphthalene-1-boronic acid (Compound A-2)

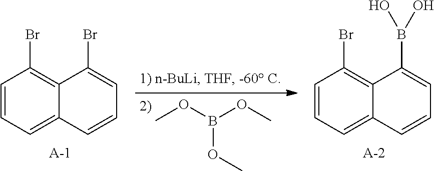

A solution of n-butyllithium in hexane (3.34 mL, 1.6 M) was added dropwise to a solution of 1,8-dibromonaphthalene (compound A-1) (1.430 g, 5.00 mmol) in THF at −60° C. over a period of 5 min. After maintaining at this temperature for 1 h, trimethyl borate (668 mL, 6.00 mmol) was added dropwise to the reaction system, which was then warmed to room temperature. After 2.5 h, a saturated aqueous solution of NH₄Cl (50 mL) was added at room temperature. After 2.5 h, the reaction system was extracted with ethyl acetate (50 mL). The organic phase was washed with saturated brine (50 mL), dried over anhydrous Na₂SO₄, and filtered. The filtrate was concentrated, and the residue was purified by silica gel column chromatography (n-hexane/acetone=7:3, v/v) to obtain compound A-2 in the form of a white solid (823.9 mg, 59% yield).

¹H-NMR (400 MHz, CD₃COCD₃): δ 7.20 (s, 2H), 7.38 (dd, J=8.0, 7.6 Hz, 1H), 7.52 (dd, J=8.0, 6.8 Hz, 1H), 7.63 (dd, J=6.8, 1.2 Hz, 1H), 7.83 (dd, J=7.6, 1.2 Hz, 1H), 7.90 (dd, J=8.0, 1.2 Hz, 1H), 7.93 (dd, J=8.0, 1.2 Hz, 1H).

LC-MS (ESI): m/z 272.7 [M+Na]⁺.

### Step 2: Synthesis of 1-bromo-8-(methyl-d₃)naphthalene (Intermediate A)

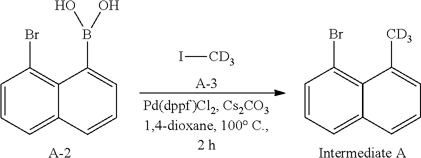

In a reaction flask, compound A-2, deuterated iodomethane (compound A-3) (1.1 eq.), Cs₂CO₃ (1.1 eq.) and Pd(dppf)Cl₂ (0.1 eq.) were added to 1,4-dioxane. After purging with nitrogen, the resulting mixture was heated to 100° C. The completion of the reaction was detected by TLC. After the completion of the reaction, the reaction system was cooled to room temperature. A saturated aqueous solution of sodium chloride was added, followed ethyl acetate for extraction. The organic phase was washed with saturated brine, dried over anhydrous Na₂SO₄, and filtered. The filtrate was concentrated, and the residue was purified by silica gel column chromatography (hexane/acetone=7:3, v/v) to obtain intermediate A in the form of a white solid.

¹H-NMR (400 MHz, CDCl₃): δ 7.81 (dd, J=7.4, 1.3 Hz, 1H), 7.76 (dd, J=8.1, 1.0 Hz, 1H), 7.66-7.59 (m, 1H), 7.30-7.22 (m, 2H), 7.19 (m, 1H).

LC-MS (ESI): m/z 224.0 [M+H]⁺.

In addition, intermediate A can also be synthesized through the following scheme.

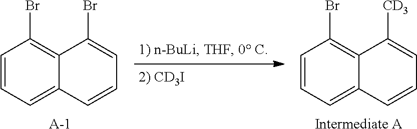

To a solution of 1,8-dibromonaphthalene (compound A-1) (1 g, 3.50 mmol) in THF (20 mL) was added a solution of n-butyllithium in pentane (2.62 mL, 1.6 M, 4.2 mmol) at 0° C. After stirring at this temperature for 30 min, deuterated iodomethane (3.05 g, 21 mmol) was added dropwise. The resulting mixture was heated to 25° C. and stirred for 3 h. The reaction system was quenched with water (50 mL) and extracted with ethyl acetate (50 mL×3). The organic phase was washed with saturated brine (20 mL), dried over anhydrous Na₂SO₄, and filtered. The filtrate was concentrated under reduced pressure, and the residue was purified by silica gel column chromatography (ethyl acetate/petroleum ether, ethyl acetate %=0-1%, v/v) to obtain intermediate A (300 mg, 38% yield).

### Intermediate Preparation Example 2: Synthesis of Intermediates C Through G

With a method similar to that in Intermediate Preparation Example 1, key intermediates shown in Table 1 were obtained from corresponding compound A-1 and compound A-3.

TABLE 1
A list of key intermediates

| | | | LC-MS |
|---|---|---|---|
| No. | Structural formulas | Name | [M + H]⁺ |
| Intermediate C | 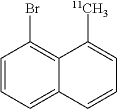 | 1-bromo-8-(methyl-¹¹C)naphthalene | 220.0 |
| Intermediate D | 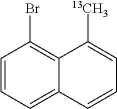 | 1-bromo-8-(methyl-¹³C)naphthalene | 222.0 |
| Intermediate E | 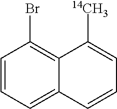 | 1-bromo-8-(methyl-¹⁴C)naphthalene | 223.0 |
| Intermediate F | 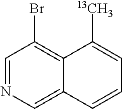 | 4-bromo-5-(methyl-¹³C)isoquinoline | 223.0 |
| Intermediate G | 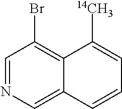 | 4-bromo-5-(methyl-¹⁴C)isoquinoline | 224.0 |

### Intermediate Preparation Example 3: Synthesis of Benzyl (S)-2-(cyanomethyl)-4-(2-(((S)-1-methylpyrrolidin-2-yl)methoxy)-5,6,7,8-tetrahydropyrido[3,4-c]pyrimidin-4-yl)piperazine-1-carboxylate (Intermediate B)

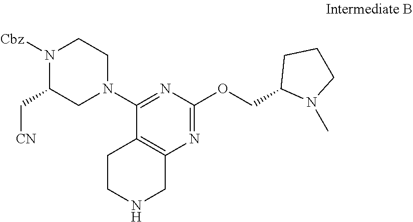 [SMILES: CN1CCC[C@H]1COc1nc2c(c(N3CCN(C(=O)OCc4ccccc4)[C@@H](CC#N)C3)n1)CCNC2]

### Step 1: Synthesis of 4-hydroxy-2-(methylthio)-5,8-dihydropyrido[3,4-d]pyrimidine-7(8H)-carboxylicacidtert-butylester (Compound B-2)

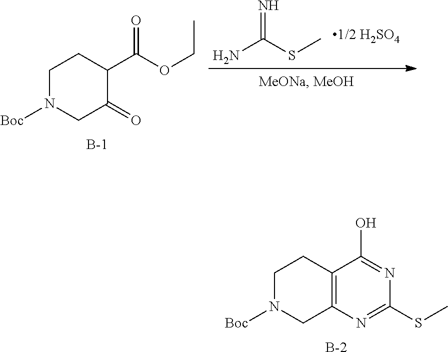

To a solution of 1-(tert-butyl) 4-ethyl 3-oxopiperidine-1,4-dicarboxylate (compound B-1) (25 g, 92.1 mmol) in MeOH (500 mL) was added MeONa (24.9 g, 460 mmol), followed by S-methylisothiourea hemisulfate (46.2 g, 166 mmol), at room temperature under a nitrogen atmosphere. The reaction mixture was stirred at ambient temperature overnight. The reaction system was adjusted to pH 5 with 2 M HCl and concentrated under reduced pressure to remove MeOH. The residue was suspended in ethyl acetate (150 mL) and water (150 mL), and the resulting suspension was stirred rapidly. The suspension was filtered to collect a white solid. The filtrate was separated, and the organic phase was washed with water (150 mL) and saturated brine (100 mL). The organic phase was dried over anhydrous Na₂SO₄, filtered and concentrated to obtain compound B-2 in the form of a white solid (24.5 g, 89.2% yield, 85.3% purity), which was used in the next step without further purification.

### Step 2: Synthesis of tert-butyl 2-(methylthio)-4-(trifluoromethanesulfonyloxy)-5,6-dihydropyrido[3,4-d]pyrimidine-7(8H)-carboxylate (Compound B-3)

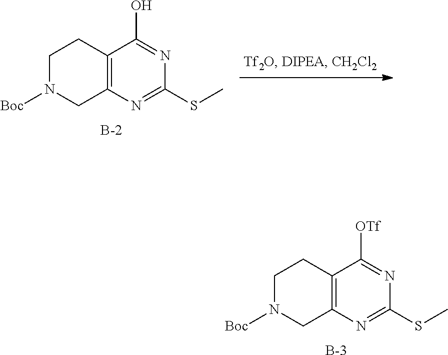

To a suspension of compound B-2 (21.0 g, 70 mmol) in dichloromethane (500 mL) was added DIPEA (18.0 g, 140 mmol), followed by triflic anhydride (29.6 g, 105 mmol, 17 mL) at 0° C. under a nitrogen atmosphere to form a brown solution immediately, which was then stirred at ambient temperature overnight. The reaction system was concentrated to obtain a brown oil. The oil was purified by silica gel column chromatography (ethyl acetate/petroleum ether, ethyl acetate %=0-20%, v/v) to obtain compound B-3 in the form of a yellow solid (20 g, 66.4% yield).

### Step 3: Synthesis of tert-butyl (S)-4-(4-(benzyloxycarbonyl)-3-(cyanomethyl)piperazin-1-yl)-2-(methylthio)-5,6-dihydropyrido [3,4-d]pyrimidine-7(8H)-carboxylate (Compound B-4)

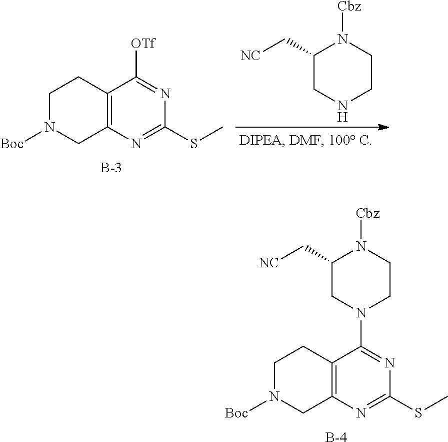

Compound B-3 (8.5 g, 19.8 mmol) and benzyl (S)-2-(cyanomethyl)piperazine-1-carboxylate (5.6 g, 21.8 mmol) were stirred in a solution of DIPEA (7.66 g, 59.4 mmol) in DMF (100 mL) at 100° C. under a nitrogen atmosphere. After the completion of the reaction, the solvent was removed in vacuo. The residue was purified by silica gel column chromatography (ethyl acetate/petroleum ether, ethyl acetate %=0-20%, v/v) to obtain compound B-4 in the form of a yellow solid (10.5 g, 98% yield).

### Step 4: Synthesis of tert-butyl 4-((S)-4-(benzyloxycarbonyl)-3-(cyanomethyl)piperazin-1-yl)-2-(methylsulfinyl)-5,6-dihydropyrido[3,4-d]pyrimidine-7(8H)-carboxylate (Compound B-5)

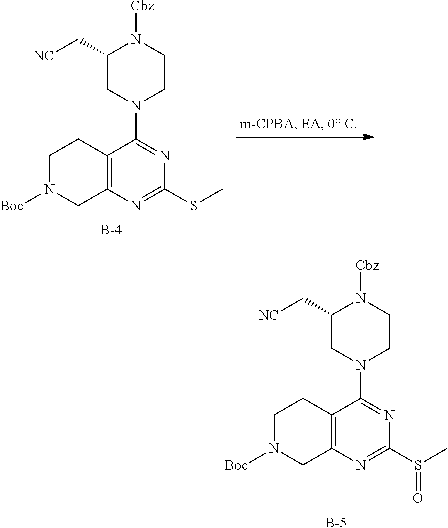

To a solution of compound B-4 (10 g, 18.6 mmol) in EtOAc (500 mL) was added m-chloroperoxybenzoic acid (m-CPBA) (4.17 g, 24.2 mmol) in portions at 0° C. After being stirring for 2 h, the mixture was diluted with water (800 mL) and adjusted to pH 8 with a saturated aqueous solution of NaHCO₃. The organic and aqueous phases were isolated, and the aqueous phase was extracted twice with EtOAc. The organic phases were pooled, dried over anhydrous Na₂SO₄, and concentrated. The residue was purified by silica gel column chromatography (methanol/ethyl acetate, methanol %=0-10%, v/v) to obtain compound B-5 in the form of a white solid (10 g, 97% yield).

### Step 5: Synthesis of tert-butyl 4-((S)-4-(benzyloxycarbonyl)-3-(cyanomethyl)piperazin-1-yl)-2-(((S)-1-methylpyrrolidin-2-yl)methoxy)-5,6-dihydropyrido[3,4-d]pyrimidine-7(8H)-carboxylate (Compound B-6)

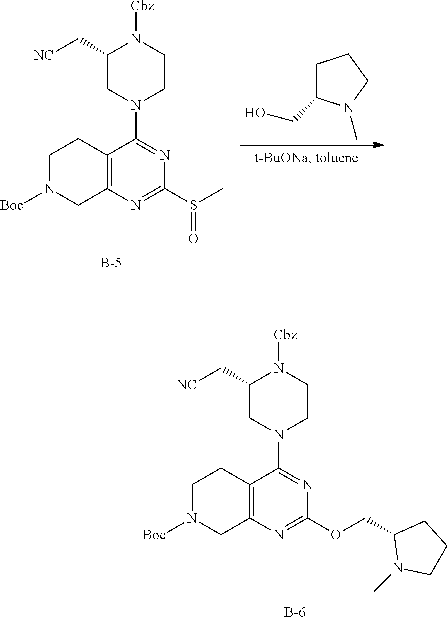

To a solution of compound B-5 (10 g, 18.05 mmol) and (S)-(1-methylpyrrolidin-2-yl)methanol (3.6 g, 31.66 mmol) in toluene (300 mL) was added t-BuONa (3.7 g, 36.1 mmol). The resulting mixture was stirred overnight at 120° C. under a nitrogen atmosphere. The reaction mixture was cooled and diluted with EtOAc (200 mL) and water (100 mL). The organic phase was separated and washed with saturated brine (50 mL), dried over anhydrous Na₂SO₄ and concentrated. The residue was purified by silica gel column chromatography (methanol/ethyl acetate, methanol %=0-10%, v/v) to obtain compound B-6 (8.6 g, 79% yield).

### Step 6: Synthesis of benzyl (S)-2-(cyanomethyl)-4-(2-(((S)-1-methylpyrrolidin-2-yl)methoxy)-5,6,7,8-tetrahydropyrido[3,4-d]pyrimidin-4-yl)piperazine-1-carboxylate (Intermediate B)

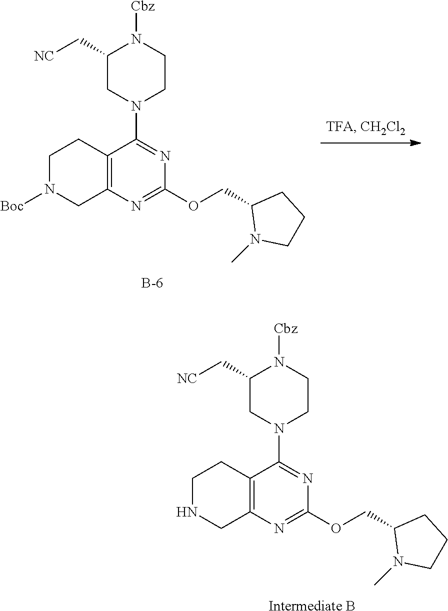

Compound B-6 (8.5 g, 14.04 mmol) was dissolved in DCM (50 mL), and TFA (50 mL) was added. The resulting mixture was stirred at 0° C. for 2 h. The reaction mixture was poured into aqueous ammonia, and the aqueous phase was extracted twice with DCM. The organic phases were pooled, dried over anhydrous Na₂SO₄ and concentrated to obtain intermediate B in the form of a brown solid (6 g, 84.5% yield).

LC-MS (ESI): m/z 506.3 [M+H]±.

### Intermediate Preparation Example 4: Synthesis of Intermediates H Through I

With a method similar to that in Intermediate Preparation Example 3, key intermediates shown in Table 2 were obtained by replacing benzyl (S)-2-(cyanomethyl)piperazine-1-carboxylate in step 3 with the corresponding material.

TABLE 2
A list of key intermediates

| | | | LC-MS |
|---|---|---|---|
| No. | Structural formulas | Name | [M + H]⁺ |
| Intermediate H | 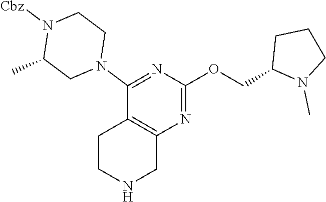 [SMILES: C[C@H]1CN(c2nc(OC[C@@H]3CCCN3C)nc3c2CCNC3)CCN1C(=O)OCc1ccccc1] | Benzyl (S)-2-methyl-4-(2-(((S)-1- methylpyrrolidin-2-yl) methoxy)-5,6,7,8-tetrahydropyrido[3,4-d] pyrimidin-4-yl)piperazine-1-carboxylate | 480.3 |
| Intermediate I | 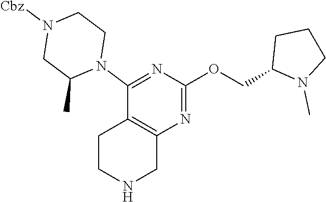 [SMILES: C[C@H]1CN(C(=O)OCc2ccccc2)CCN1c1nc(OC[C@@H]2CCCN2C)nc2c1CCNC2] | (S)-3-methyl-4-(2-(((S)-1-methylpyrrolidin- 2-yl) methoxy)-5,6,7,8-tetrahydropyrido[3,4-d] pyrimidin-4-yl)piperazine-1-carboxylate | 480.3 |

### Intermediate Preparation Example 5: Synthesis of (S)-(1-(methyl-d₃)pyrrolidin-2-yl)methanol (Intermediate J)

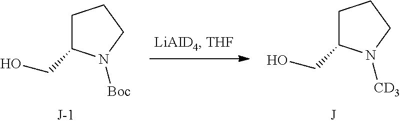 [SMILES: CC(C)(C)OC(=O)N1CCC[C@H]1CO ; [2H]C([2H])([2H])N1CCC[C@H]1CO]

To a solution of tert-butyl (S)-2-(hydroxymethyl)pyrrolidine-1-carboxylate (2.0 g, 9.95 mmol) in anhydrous THF (60 mL) was added lithium aluminum deuteride (1.25 g, 30.0 mmol) in portions at 0° C. After the completion of the addition, the reaction system was heated to 65° C., stirred for 2 h, and then quenched with Na₂SO₄·10 H₂O. The reaction system was filtered, and filter cake was washed with THF. The filtrates were pooled, dried over anhydrous Na₂SO₄ and concentrated to obtain intermediate J in the form of a colorless oil (500 mg, 43% yield).

LC-MS (ESI): m/z 119.1 [M+H]⁺.

### Intermediate Preparation Example 5: Synthesis of Intermediates K Through P

With a method similar to that in Intermediate Preparation Example 5, key intermediates shown in Table 3 were obtained.

TABLE 3
A list of key intermediates

| | | | LC-MS |
|---|---|---|---|
| No. | Structural formulas | Name | [M + H]⁺ |
| Intermediate K | 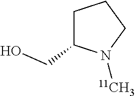 | (S)-(1-(methyl-¹¹C)pyrrolidin-2-yl)methanol | 115.1 |
| Intermediate L | 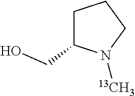 | (S)-(1-(methyl-¹³C)pyrrolidin-2-yl)methanol | 117.1 |
| Intermediate M | 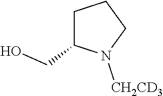 | (S)-(1-(ethyl-2,2,2-d₃)pyrrolidin-2-yl)methanol | 133.1 |
| Intermediate N | 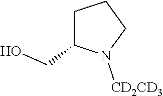 | (S)-(1-(ethyl-d₅)pyrrolidin-2-yl)methanol | 135.1 |
| Intermediate O | 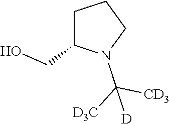 [SMILES: [2H]C([2H])([2H])C([2H])(N1CCC[C@H]1CO)C([2H])([2H])[2H]] | (S)-(1-(isopropyl-d₇)pyrrolidin-2-yl)methanol | 151.1 |
| Intermediate P | 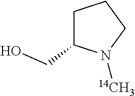 | (S)-(1-(methyl-¹⁴C)pyrrolidin-2-yl)methanol | 118.1 |

### Intermediate Preparation Example 6: Synthesis of Intermediates Q Through Y

With a method similar to that in Intermediate Preparation Example 3 or 4, key intermediates shown in Table 4 were obtained based on intermediates J through P.

TABLE 4
A list of key intermediates

| | | | LC-MS |
|---|---|---|---|
| No. | Structural formulas | Name | [M + H]⁺ |
| Intermediate Q | 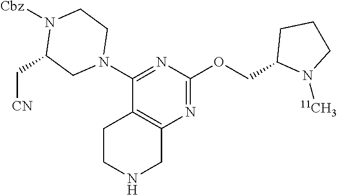 [SMILES: [11CH3]N1CCC[C@H]1COc1nc2c(c(N3CCN(C(=O)OCc4ccccc4)[C@@H](CC#N)C3)n1)CCNC2] | Benzyl (S)-2-(cyanomethyl)-4-(2-(((S)-1- (methyl-¹¹C)pyrrolidin-2-yl) methoxy)-5,6,7,8-tetrahydropyrido[3,4-d] pyrimidin-4-yl)piperazine-1-carboxylate | 505.3 |
| Intermediate R | 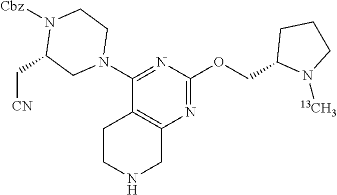 [SMILES: [13CH3]N1CCC[C@H]1COc1nc2c(c(N3CCN(C(=O)OCc4ccccc4)[C@@H](CC#N)C3)n1)CCNC2] | Benzyl (S)-2-(cyanomethyl)-4-(2-(((S)-1- (methyl-¹³C)pyrrolidin-2-yl) methoxy)-5,6,7,8-tetrahydropyrido[3,4-d] pyrimidin-4-yl)piperazine-1-carboxylate | 507.3 |
| Intermediate S | 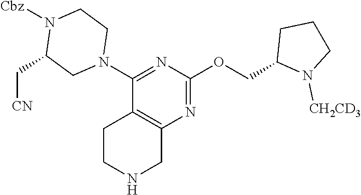 | Benzyl (S)-2-(cyanomethyl)-4-(2-(((S)-1- (ethyl-2,2,2-d₃)pyrrolidin-2-yl) methoxy)-5,6,7,8-tetrahydropyrido[3,4-d] pyrimidin-4-yl)piperazine-1-carboxylate | 523.3 |
| Intermediate T | 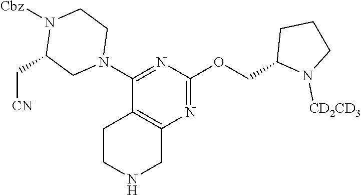 | Benzyl (S)-2-(cyanomethyl)-4-(2-(((S)-1- (ethyl-d₅)pyrrolidin-2-yl) methoxy)-5,6,7,8-tetrahydropyrido[3,4-d] pyrimidin-4-yl)piperazine-1-carboxylate | 525.3 |
| Intermediate U | 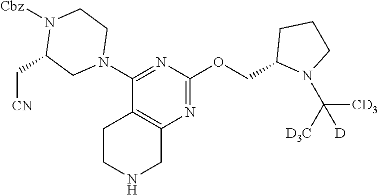 [SMILES: [2H]C([2H])([2H])C([2H])(N1CCC[C@H]1COc1nc2c(c(N3CCN(C(=O)OCc4ccccc4)[C@@H](CC#N)C3)n1)CCNC2)C([2H])([2H])[2H]] | Benzyl (S)-2-(cyanomethyl)-4-(2-(((S)-1- (isopropyl-d₇)pyrrolidin-2-yl) methoxy)-5,6,7,8-tetrahydropyrido[3,4-d] pyrimidin-4-yl)piperazine-1-carboxylate | 541.4 |
| Intermediate V | 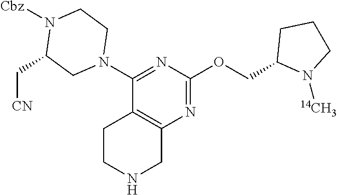 [SMILES: [14CH3]N1CCC[C@H]1COc1nc2c(c(N3CCN(C(=O)OCc4ccccc4)[C@@H](CC#N)C3)n1)CCNC2] | Benzyl (S)-2-(cyanomethyl)-4-(2-(((S)-1- (methyl-¹⁴C)pyrrolidin-2-yl) methoxy)-5,6,7,8-tetrahydropyrido[3,4-d] pyrimidin-4-yl)piperazine-1-carboxylate | 508.3 |
| Intermediate W | 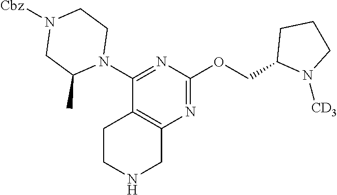 [SMILES: [2H]C([2H])([2H])N1CCC[C@H]1COc1nc2c(c(N3CCN(C(=O)OCc4ccccc4)C[C@@H]3C)n1)CCNC2] | Benzyl (S)-3-methyl-4-(2-(((S)-1-(methyl- d₃)pyrrolidin-2-yl) methoxy)-5,6,7,8-tetrahydropyrido[3,4-d] pyrimidin-4-yl)piperazine-1-carboxylate | 484.3 |
| Intermediate | | Benzyl (S)-2-methyl-4-(2-(((S)-1-(methyl- | 484.3 |
| X | | d₃)pyrrolidin-2-yl) | |
| | | methoxy)-5,6,7,8-tetrahydropyrido[3,4-d] | |
| | | pyrimidin-4-yl)piperazine-1-carboxylate | |
| Intermediate | | Benzyl (S)-2-(cyanomethyl)-4-(2-(((S)-1- | 509.5 |
| Y | | (methyl-d₃)pyrrolidin-2-yl) | |
| | | methoxy)-5,6,7,8-tetrahydropyrido[3,4-d] | |
| | | pyrimidin-4-yl)piperazine-1-carboxylate | |

The following examples are intended to provide a better understanding of the present invention rather than limit the present invention.

### Example 1: Synthesis of 2-((S)-1-acryloyl-4-(7-(8-(methyl-d₃)naphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Compound 1)

### Step 1: Synthesis of benzyl (S)-2-(cyanomethyl)-4-(7-(8-(methyl-d₃)naphthalen-1-yl)-2-(((S)-1-methylpyrrolidin-2-yl) methoxy)-5,6,7,8-tetrahydropyrido[3,4-d]pyrimidin-4-yl)piperazine-1-carboxylate (Compound I-B-1)

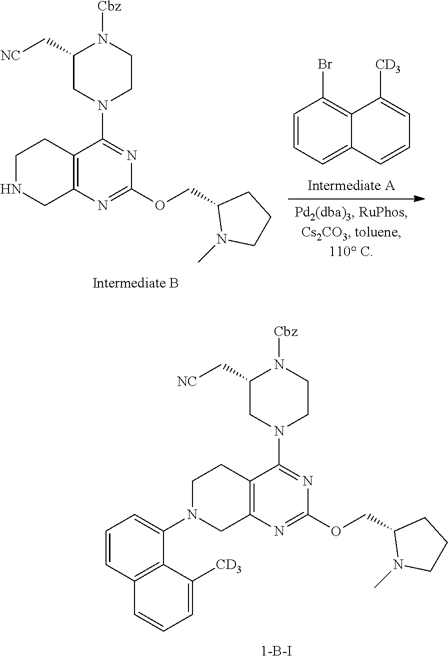 [SMILES: CN1CCC[C@H]1COc1nc2c(c(N3CCN(C(=O)OCc4ccccc4)[C@@H](CC#N)C3)n1)CCNC2 ; [2H]C([2H])([2H])c1cccc2cccc(Br)c12 ; "Intermediate B" ; [2H]C([2H])([2H])c1cccc2cccc(N3CCc4c(nc(OC[C@@H]5CCCN5C)nc4N4CCN(C(=O)OCc5ccccc5)[C@@H](CC#N)C4)C3)c12 ; "1-B-I"]

To a solution of intermediate B (4 g, 7.92 mmoL) and intermediate A (2.3 g, 10.3 mmoL) in toluene (150 mL) were added Cs₂CO₃ (7.7 g, 23.8 mmoL), RuPhos (739 mg, 1.584 mmoL) and Pd₂(dba)₃ (725 mg, 0.792 mmoL). Nitrogen was introduced into the reaction mixture, which was then heated to reflux and stirred overnight. The reaction mixture was filtered, and the filtrate was concentrated. The residue was purified by silica gel column chromatography (ethyl acetate/petroleum ether, ethyl acetate %=0-100%, v/v) to obtain compound I-B-1 (2.3 g, 45% yield).

LC-MS (ESI): m/z 649.4 [M+H]⁺.

### Step 2: Synthesis of 2-((S)-4-(7-(8-(methyl-d₃)naphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Compound I-B-2)

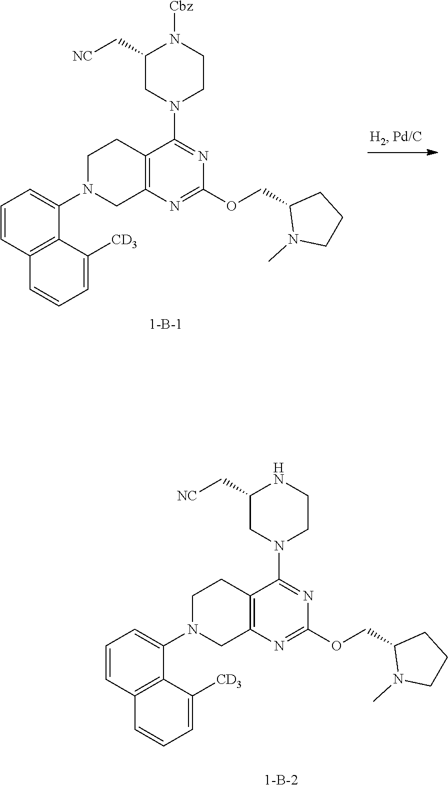 [SMILES: [2H]C([2H])([2H])c1cccc2cccc(N3CCc4c(nc(OC[C@@H]5CCCN5C)nc4N4CCN(C(=O)OCc5ccccc5)[C@@H](CC#N)C4)C3)c12 ; [2H]C([2H])([2H])c1cccc2cccc(N3CCc4c(nc(OC[C@@H]5CCCN5C)nc4N4CCN[C@@H](CC#N)C4)C3)c12]

Compound I-B-1 (2.3 g, 3.56 mmol) and 10% Pd/C (800 mg) were added to methanol (100 mL), and the resulting mixture was stirred overnight at ambient temperature under a hydrogen atmosphere. The mixture was filtered, and the filtrate was concentrated to obtain compound I-B-2 in the form of a yellow solid (1.9 g, 99% yield).

LC-MS (ESI): m/z 515.3 [M+H]⁺.

### Step 3: Synthesis of 2-((S)-1-acryloyl-4-(7-(8-(methyl-d₃)naphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Compound 1)

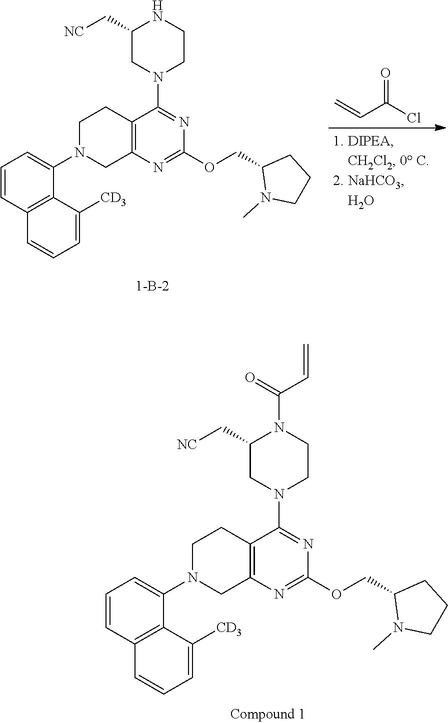

To a solution of compound I-B-2 (1.9 g, 3.72 mmol) and DIPEA (1.44 g, 11.16 mmol) in DCM (50 mL) was added a solution of acryloyl chloride (370 mg, 4.09 mmol) in DCM (10 mL) with ice water cooling. The mixture was stirred at 0° C. for 2 h, then quenched with a saturated aqueous solution of sodium bicarbonate, and extracted with DCM (100 mL×2). The organic phases were pooled, dried over anhydrous sodium sulfate, and concentrated. The residue was purified by preparative HPLC (Waters high performance liquid system; Shimadzu Inertsil ODS-3 chromatography column (10 μm, 20×250 nm), mobile phase: purified water (containing 0.1% formic acid, v/v)/acetonitrile, from 35% to 50% (v/v)) to obtain compound 1 (1.27 g, 60% yield).

¹H-NMR (400 MHz, DMSO): δ7.76 (d, J=8.1 Hz, 1H), 7.74-7.66 (m, 1H), 7.49-7.43 (m, 1H), 7.34-7.26 (m, 3H), 6.86 (brs, 1H), 6.19 (d, J=16.5 Hz, 1H), 5.78 (d, J=12.4 Hz, 1H), 4.97-4.78 (m, 1H), 4.41-4.38 (m, 1H), 4.23 (dd, J=10.7, 4.8 Hz, 1H), 4.07-4.94 (m, 4H), 3.78-3.66 (m, 2H), 3.45-3.42 (m, 2H), 3.13-3.02 (m, 4H), 2.96-2.87 (m, 2H), 2.77-2.67 (m, 1H), 2.35 (s, 3H), 2.19-2.12 (m, 1H), 1.96-1.87 (m, 1H), 1.75-1.54 (m, 3H).

LC-MS (ESI): m/z 569.3 [M+H]⁺.

### Example 2: Synthesis of Compounds 2 Through 16

With a method similar to that in Example 1, compounds shown in Table 5 were obtained from corresponding intermediate A, intermediate B, intermediate C, intermediate D, intermediate E, intermediate F, intermediate G (or 4-bromo-5-(methyl-d₃)isoquinoline, 4-bromo-5-(methyl-¹¹C)isoquinoline) and (substituted) acryloyl chloride (e.g. 2-fluoroacryloyl chloride or (E)-4-(cyclopropylamino)-2-butenoyl chloride).

TABLE 5
Identification data for compounds 2 through 16

| | LC-MS |
|---|---|
| No. | [M + 1]⁺ |
| Compound 2 | 587.3 |
| Compound 3 | 613.4 |
| Compound 4 | 570.3 |
| Compound 5 | 562.3 |
| Compound 6 | 565.3 |
| Compound 7 | 562.3 |
| Compound 8 | 544.3 |
| Compound 9 | 583.3 |
| Compound 10 | 567.3 |
| Compound 11 | 585.3 |
| Compound 12 | 568.3 |
| Compound 13 | 586.3 |
| Compound 14 | 587.3 |
| Compound 15 | 568.3 |
| Compound 16 | 586.3 |

### Example 3: Synthesis of 2-((S)-1-acryloyl-4-(7-(8-(methyl-d₃)naphthalen-1-yl)-2-(((S)-1-(methyl-d₃)pyrrolidin-2-yl)methoxy)-5,6,7,8-tetrahydropyrido[3,4-d]pyrimidin-4-yl)piperazin-2-yl)acetonitrile (Compound 17)

### Step 1: Synthesis of benzyl (S)-2-(cyanomethyl)-4-(7-(8-(methyl-d₃)naphthalen-1-yl)-2-(((S)-1-(methyld₃)pyrrolidin-2-yl) methoxy)-5,6,7,8-tetrahydropyrido[3,4-d]pyrimidin-4-yl)piperazine-1-carboxylate (Compound 17-Y-1)

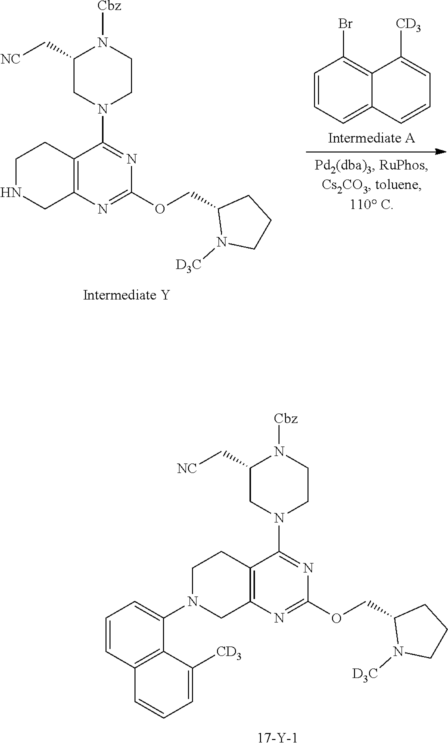 [SMILES: [2H]C([2H])([2H])N1CCC[C@H]1COc1nc2c(c(N3CCN(C(=O)OCc4ccccc4)[C@@H](CC#N)C3)n1)CCNC2 ; [2H]C([2H])([2H])c1cccc2cccc(Br)c12 ; [2H]C([2H])([2H])c1cccc2cccc(N3CCc4c(nc(OC[C@@H]5CCCN5C([2H])([2H])[2H])nc4N4CCN(C(=O)OCc5ccccc5)[C@@H](CC#N)C4)C3)c12]

To a solution of intermediate Y (4 g, 7.92 mmoL) and intermediate A (2.3 g, 10.3 mmoL) in toluene (150 mL) were added Cs₂CO₃ (7.7 g, 23.8 mmoL), RuPhos (739 mg, 1.584 mmoL) and Pd₂(dba)₃ (725 mg, 0.792 mmoL). Nitrogen was introduced into the reaction mixture, which was then heated to reflux and stirred overnight. The reaction mixture was filtered, and the filtrate was concentrated. The residue was purified by silica gel column chromatography (ethyl acetate/petroleum ether, ethyl acetate %=0-100%, v/v) to obtain compound 17-Y-1 (2.3 g, 45% yield).

LC-MS (ESI): m/z 652.4 [M+H]⁺.

### Step 2: Synthesis of 2-(S)-4-(7-(8-(methyl-d₃)naphthalen-1-yl)-2-(((S)-1-(methyld₃)pyrrolidin-2-yl)methoxy)-5,6,7,8-tetrahydropyrido[3,4-d]pyrimidin-4-yl)piperazin-2-yl)acetonitrile (Compound 17-Y-2)

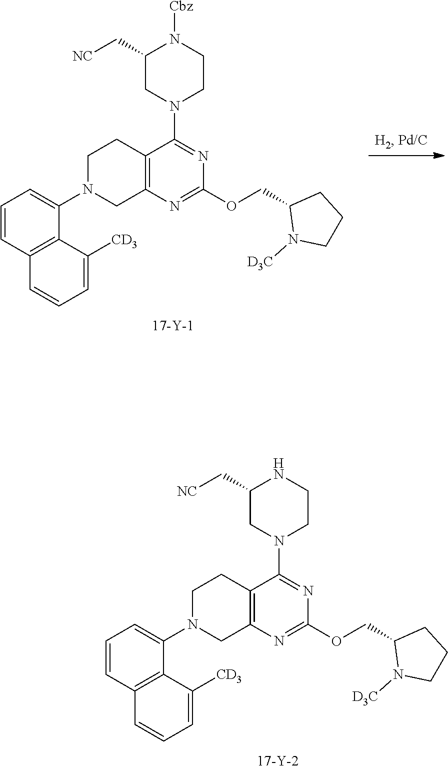 [SMILES: [2H]C([2H])([2H])c1cccc2cccc(N3CCc4c(nc(OC[C@@H]5CCCN5C([2H])([2H])[2H])nc4N4CCN(C(=O)OCc5ccccc5)[C@@H](CC#N)C4)C3)c12 ; [2H]C([2H])([2H])c1cccc2cccc(N3CCc4c(nc(OC[C@@H]5CCCN5C([2H])([2H])[2H])nc4N4CCN[C@@H](CC#N)C4)C3)c12]

Compound 17-Y-1 (2.3 g, 3.56 mmol) and 10% Pd/C (800 mg) were added to methanol (100 mL), and the resulting mixture was stirred overnight at ambient temperature under a hydrogen atmosphere. The mixture was filtered, and the filtrate was concentrated to obtain compound 17-Y-2 in the form of a yellow solid (1.9 g, 99% yield).

LC-MS (ESI): m/z 518.3 [M+H]⁺.

### Step 3: Synthesis of 2-((S)-1-acryloyl-4-(7-(8-(methyl-d₃)naphthalen-1-yl)-2-(((S)-1-(methyld₃)pyrrolidin-2-yl)methoxy)-5,6,7,8-tetrahydropyrido[3,4-d]pyrimidin-4-yl)piperazin-2-yl)acetonitrile (Compound 17)

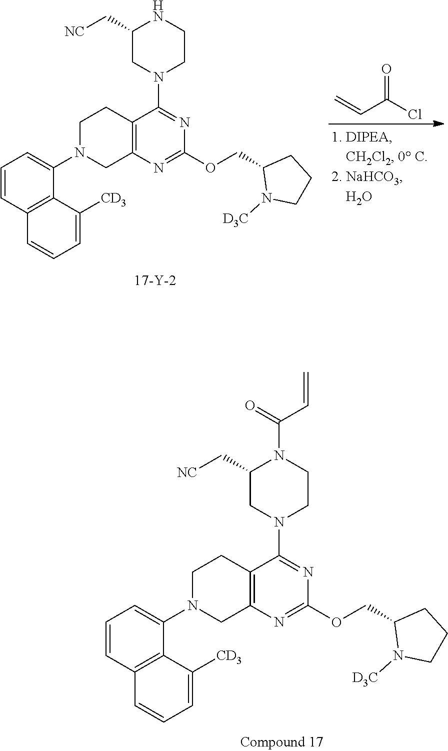

To a solution of compound 17-Y-2 (1.9 g, 3.72 mmol) and DIPEA (1.44 g, 11.16 mmol) in DCM (50 mL) was added a solution of acryloyl chloride (370 mg, 4.09 mmol) in DCM (10 mL) with ice water cooling. The mixture was stirred at 0° C. for 2 h, then quenched with a saturated aqueous solution of sodium bicarbonate, and extracted with DCM (100 mL×2). The organic phases were pooled, dried over anhydrous sodium sulfate, and concentrated. The residue was purified by preparative HPLC (Waters high performance liquid system; Shimadzu Inertsil ODS-3 chromatography column (10 μm, 20×250 nm), mobile phase: purified water (containing 0.1% formic acid, v/v)/acetonitrile, acetonitrile %=35%-50% (v/v)) to obtain compound 17 (1.27 g, 60% yield).

¹H-NMR (400 MHz, DMSO): δ7.76 (d, J=8.1 Hz, 1H), 7.71-7.68 (m, 1H), 7.47-7.45 (m, 1H), 7.39-7.26 (m, 3H), 6.86 (brs, 1H), 6.19 (d, J=16.6 Hz, 1H), 5.78 (d, J=12.4 Hz, 1H), 5.05-4.70 (m, 1H), 4.50-4.37 (m, 2H), 4.20-3.94 (m, 5H), 3.78-3.66 (m, 2H), 3.33-3.43 (m, 1H), 3.13-3.02 (m, 6H), 2.94-2.91 (m, 1H), 2.76-2.67 (m, 1H), 2.45-2.20 (m, 1H), 2.19-2.12 (m, 1H), 1.95-1.87 (m, 1H), 1.67-1.54 (m, 3H).

LC-MS (ESI): m/z 572.3 [M+H]⁺.

### Example 4: Synthesis of 2-((S)-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)-1-(2-fluoroacryloyl)piperazin-2-yl)acetonitrile (Compound 18)

### Step 1: Synthesis of benzyl (S)-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)-2-(cyanomethyl)piperazine-1-carboxylate (Compound 18-Y-1)

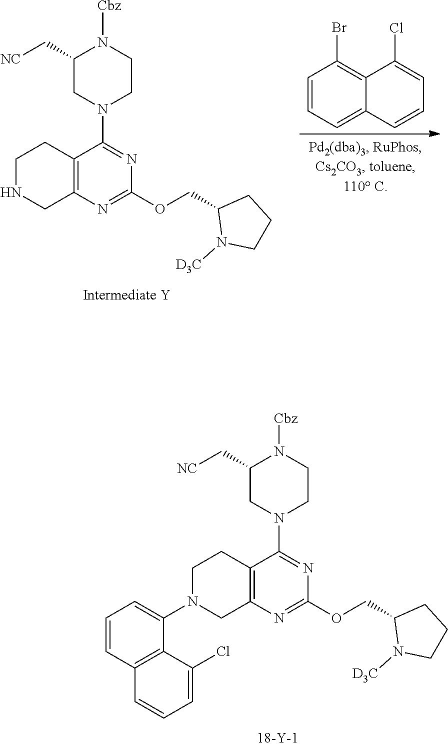 [SMILES: [2H]C([2H])([2H])N1CCC[C@H]1COc1nc2c(c(N3CCN(C(=O)OCc4ccccc4)[C@@H](CC#N)C3)n1)CCNC2 ; Clc1cccc2cccc(Br)c12 ; [2H]C([2H])([2H])N1CCC[C@H]1COc1nc2c(c(N3CCN(C(=O)OCc4ccccc4)[C@@H](CC#N)C3)n1)CCN(c1cccc3cccc(Cl)c13)C2]

To a solution of intermediate Y (400 mg, 0.79 mmol) and 1-bromo-8-chloronaphthalene (384 mg, 1.6 mmol) in toluene were added Cs₂CO₃ (1.0 g, 3.2 mmol), RuPhos (40 mg, 0.086 mmol) and Pd₂(dba)₃ (40 mg, 0.044 mmol). Nitrogen was introduced twice into the reaction mixture, which was then heated to reflux and stirred overnight. The reaction mixture was filtered, and the filtrate was concentrated. The residue was purified by silica gel column chromatography (methanol/dichloromethane, dichloromethane %=0-5%, v/v) to obtain compound 18-Y-1 (280 mg, 53% yield).

LC-MS (ESI): m/z 669.3 [M+H]⁺.

### Step 2: Synthesis of 2-((S)-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)piperazin-2-yl)acetonitrile (Compound 18-Y-2)

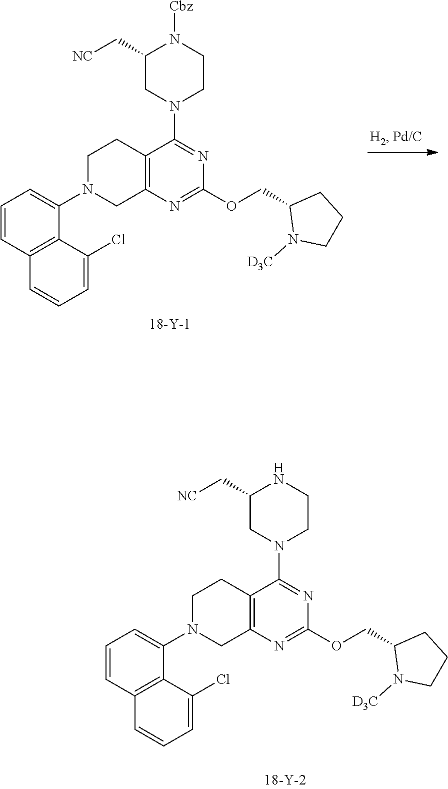 [SMILES: [2H]C([2H])([2H])N1CCC[C@H]1COc1nc2c(c(N3CCN(C(=O)OCc4ccccc4)[C@@H](CC#N)C3)n1)CCN(c1cccc3cccc(Cl)c13)C2 ; [2H]C([2H])([2H])N1CCC[C@H]1COc1nc2c(c(N3CCN[C@@H](CC#N)C3)n1)CCN(c1cccc3cccc(Cl)c13)C2]

Compound 18-Y-1 (140 mg, 0.21 mmol) and 10% Pd/C (30 mg) were added to methanol (10 mL), and the resulting mixture was stirred at 40° C. under a hydrogen atmosphere for 2 h. The mixture was filtered, and the filtrate was concentrated to obtain compound 18-Y-2 in the form of a yellow solid (112 mg, 99% yield).

LC-MS (ESI): m/z 535.3 [M+H]⁺.

### Step 3: Synthesis of 2-((S)-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)-1-(2-fluoroacryloyl)piperazin-2-yl)acetonitrile (Compound 18)

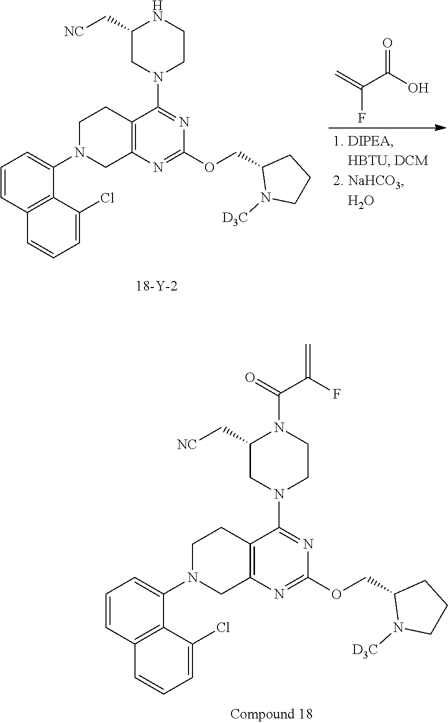

To a solution of compound 18-Y-2 (112 mg, 0.2 mmol) and 2-fluoroacrylic acid (22 mg, 0.25 mmol) in DCM (10 mL) was added DIPEA (0.2 mL, 1.2 mmol) and HBTU (200 mg, 0.53 mmol) with ice water cooling. The mixture was stirred at ambient temperature for 2 h, then quenched with a saturated aqueous solution of sodium bicarbonate, and extracted with DCM (50 mL×2). The organic layers were pooled, dried over anhydrous sodium sulfate, and concentrated. The residue was purified by preparative HPLC (Waters high performance liquid system; Shimadzu Inertsil ODS-3 chromatography column (10 μm, 20×250 nm), mobile phase: purified water (containing 0.1% formic acid)/acetonitrile, acetonitrile %=45%-50%, v/v) to obtain compound 18 (10 mg, 8% yield).

¹H-NMR (400 MHz, DMSO): δ 7.92 (d, J=8.0 Hz, 1H), 7.74 (dd, J=8.0, 4.1 Hz, 1H), 7.59-7.51 (m, 2H), 7.45 (t, J=7.7 Hz, 1H), 7.37-7.31 (m, 1H), 5.41-5.16 (m, 2H), 4.85 (brs, 1H), 4.24-4.04 (m, 2H), 4.08-3.84 (m, 4H), 3.81-3.71 (m, 1H), 3.51-3.46 (m, 1H), 3.25-3.19 (m, 2H), 3.15-3.06 (m, 4H), 3.00-2.91 (m, 2H), 2.72-2.69 (m, 1H), 2.20-2.13 (m, 1H), 1.97-1.88 (m, 1H), 1.70-1.54 (m, 3H). LC-MS (ESI): m/z 607.2 [M+H]⁺.

### Example 5: Synthesis of 2-((S)-1-acryloyl-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)piperazin-2-yl)acetonitrile (Compound 41)

### Step 1: Synthesis of benzyl (S)-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)-2-(cyanomethyl)piperazine-1-carboxylate (Compound 41-Y-1)

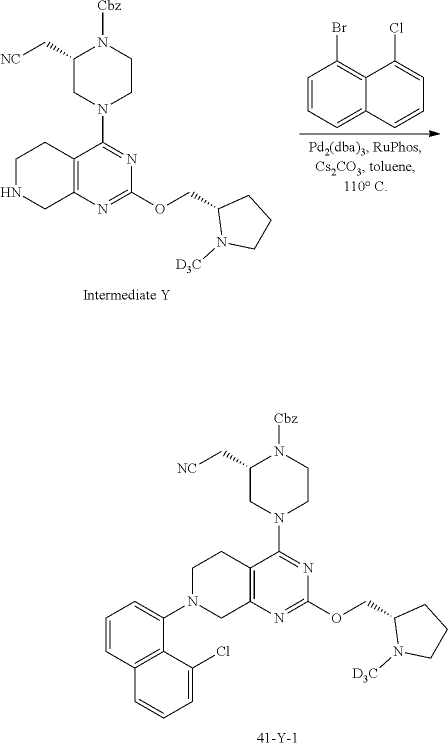 [SMILES: [2H]C([2H])([2H])N1CCC[C@H]1COc1nc2c(c(N3CCN(C(=O)OCc4ccccc4)[C@@H](CC#N)C3)n1)CCNC2 ; Clc1cccc2cccc(Br)c12 ; [2H]C([2H])([2H])N1CCC[C@H]1COc1nc2c(c(N3CCN(C(=O)OCc4ccccc4)[C@@H](CC#N)C3)n1)CCN(c1cccc3cccc(Cl)c13)C2]

To a solution of intermediate Y (400 mg, 0.79 mmol) and 1-bromo-8-chloronaphthalene (384 mg, 1.6 mmol) in toluene were added Cs₂CO₃ (1.0 g, 3.2 mmol), RuPhos (40 mg, 0.086 mmol) and Pd₂(dba)₃ (40 mg, 0.044 mmol). Nitrogen was introduced twice into the reaction mixture, which was then heated to reflux and stirred overnight. The reaction mixture was filtered, and the filtrate was concentrated. The residue was purified by silica gel column chromatography (methanol/dichloromethane, dichloromethane %=0-5%, v/v) to obtain compound 41-Y-1 (280 mg, 53% yield).

LC-MS (ESI): m/z 669.3 [MAH]⁺.

### Step 2: Synthesis of 2-((S)-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)piperazin-2-yl)acetonitrile (Compound 41-Y-2)

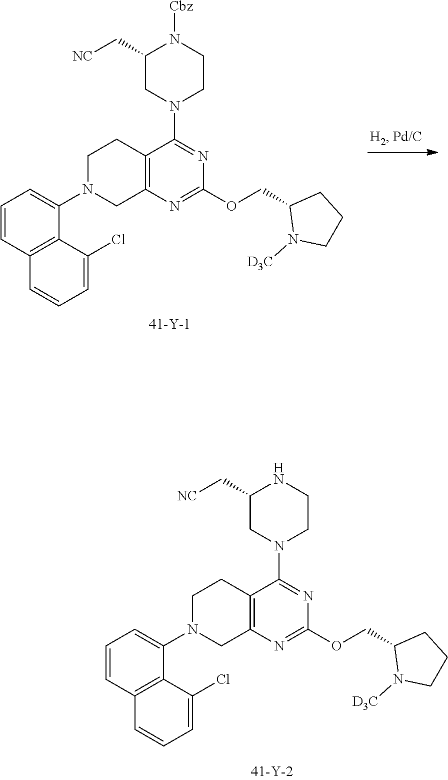 [SMILES: [2H]C([2H])([2H])N1CCC[C@H]1COc1nc2c(c(N3CCN(C(=O)OCc4ccccc4)[C@@H](CC#N)C3)n1)CCN(c1cccc3cccc(Cl)c13)C2 ; [2H]C([2H])([2H])N1CCC[C@H]1COc1nc2c(c(N3CCN[C@@H](CC#N)C3)n1)CCN(c1cccc3cccc(Cl)c13)C2]

Compound 41-Y-1 (140 mg, 0.21 mmol) and 10% Pd/C (30 mg) were added to methanol (10 mL), and the resulting mixture was stirred at 40° C. under a hydrogen atmosphere for 2 h. The mixture was filtered, and the filtrate was concentrated to obtain compound 41-Y-2 in the form of a yellow solid (112 mg, 99% yield).

LC-MS (ESI): m/z 535.3 [M+H]⁺.

### Step 3: Synthesis of 2-((S)-1-acryloyl-4-(7-(8-chloronaphthalen-1-yl)-2-(((S)-1-(methyl-d₃)pyrrolidin-2-yl)methoxy)-5,6,7,8-tetrahydropyrido[3,4-d]pyrimidin-4-yl)piperazin-2-yl)acetonitrile (Compound 41)

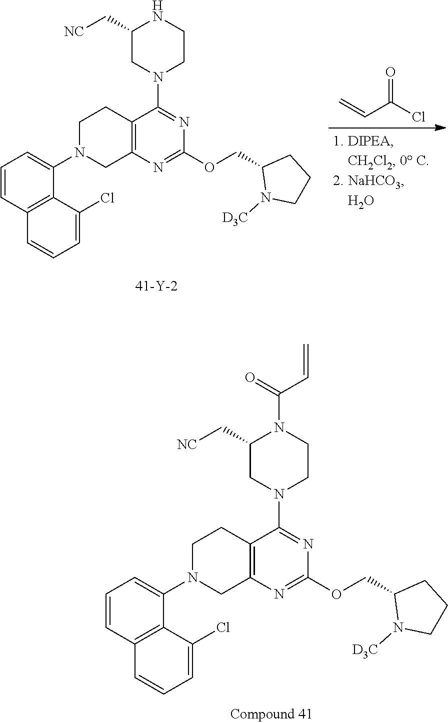

To a solution of compound 41-Y-2 (112 mg, 0.2 mmol) and DIPEA (0.2 mL, 1.2 mmol) in DCM (10 mL) was added a solution of acryloyl chloride (22 mg, 0.24 mmol) in DCM (1 mL) with ice water cooling. The mixture was stirred at 0° C. for 15 min, then quenched with a saturated aqueous solution of sodium bicarbonate, and extracted with DCM (50 mL×2). The organic phases were pooled, dried over anhydrous sodium sulfate, and concentrated. The residue was purified by preparative HPLC (Waters high performance liquid system; Shimadzu Inertsil ODS-3 chromatography column (10 μm, 20×250 nm), mobile phase: purified water (containing 0.1% formic acid, v/v)/acetonitrile, acetonitrile %=35%-45% (v/v)) to obtain compound 41 (39 mg, 33% yield).

¹H-NMR (400 MHz, DMSO): δ 7.92 (d, J=8.0 Hz, 1H), 7.74 (dd, J=8.1, 4.4 Hz, 1H), 7.63-7.50 (m, 2H), 7.45 (t, J=7.8 Hz, 1H), 7.37-7.31 (m, 1H), 6.85 (brs, 1H), 6.18 (d, J=16.7 Hz, 1H), 5.77 (d, J=12.1 Hz, 1H), 4.96-4.76 (m, 1H), 4.31-3.92 (m, 5H), 3.91-3.69 (m, 1H), 3.58-3.48 (m, 1H), 3.19-3.00 (m, 6H), 2.98-2.78 (m, 1H), 2.71-2.68 (m, 1H), 2.21-2.15 (m, 1H), 1.97-1.87 (m, 1H), 1.76-1.50 (m, 3H).

LC-MS (ESI): m/z 589.3 [M+H]⁺.

### Example 6: Synthesis of Compounds 19 Through 40 and Compound 42

With a method similar to that in Example 3, compounds shown in Table 6 were obtained from corresponding intermediates and (substituted) acryloyl chloride (e.g., 2-fluoroacryloyl chloride or (E)-4-(cyclopropylamino)-2-butenoyl chloride).

TABLE 6
Identification data for compounds 19 through 40 and Compound 42

| | LC-MS |
|---|---|
| No. | [M + 1]⁺ |
| Compound 19 | 638.4 |
| Compound 20 | 590.3 |
| Compound 21 | 547.4 |
| Compound 22 | 568.4 |
| Compound 23 | 565.4 |
| Compound 24 | 547.4 |
| Compound 25 | 565.3 |
| Compound 26 | 567.3 |
| Compound 27 | 585.3 |
| Compound 28 | 587.3 |
| Compound 29 | 589.3 |
| Compound 30 | 570.3 |
| Compound 31 | 571.3 |
| Compound 32 | 572.3 |
| Compound 33 | 583.3 |
| Compound 34 | 603.3 |
| Compound 35 | 585.3 |
| Compound 36 | 605.3 |
| Compound 37 | 601.3 |
| Compound 38 | 621.3 |
| Compound 39 | 568.3 |
| Compound 40 | 588.2 |
| Compound 42 | 569.3 |

The following biological assays can be used to test the biological activity of the compounds of the present invention.

### Example 7: ERK Protein Phosphorylation Assay

In order to examine the inhibitory activity of the compounds of the present invention against KRAS G12C protein at a cellular level, the ERK protein phosphorylation assay was selected for evaluation.

(1) H358 cells (ATCC, CRL-5807) expressing KRAS G12C protein were seeded at a concentration of 6000 cells/well in a polylysine-coated 384-well cell culture plate (Corning, BD356663), with medium components being RPMI 1640 (Gibco, A10491-01), 10% FBS (Gibco, 10099141C) and 1% Pen/Strep (Gibco, 15140-122). The cells were cultured in a 5% CO₂ cell incubator for 16 h. The serially diluted compound was added to the cell medium with Echo550 at a final DMSO concentration of 0.5%, and the incubation was continued for 3 h. Then 8% paraformaldehyde (Solarbio, P1112) was added at 40 μL/well, followed by incubation at room temperature for 20 min. After one washing with PBS, cold 100% methanol was added at 40 μL/well for permeabilization for 10 min. After one washing with PBS, a blocking solution (LI-COR, 927-40000) was added at 20 μL/well for blocking at room temperature for 1 h. Then rabbit anti-phospho-p44/42 MAPK (T202/Y204) antibody (CST, 4370S) and mouse anti-GAPDH (D4C6R) antibody (CST, 97166S) were 1:1000 and 1:2000 diluted with the blocking solution, respectively, and added to the cells at 20 μL/well for blocking at 4° C. overnight. Washing with PBST was performed 3 times, with 2 min of incubation for each washing. Then goat anti-rabbit 800CW antibody (LI-COR, 926-32211) and goat anti-mouse 680RD antibody (LI-COR, 926-68070) were 1:1000 diluted with the blocking solution, and added to the cells at 20 μL/well for incubation at room temperature for 45 min. Washing with PBST was performed 3 times, with 2 min of incubation for each washing. Finally, the cell culture plate was inverted and centrifuged at 1000 rpm for 1 min, after which fluorescence signals were read using Odyssey CLx.
(2) Data were fit by XLFit 5.0 with the 4-parameter formula Y=Bottom+(Top−Bottom)/(1+10\hat{ }((Log IC₅₀−X)×HillSlope)) to calculate IC₅₀ values, with the results shown in Table 7, where “A” indicates IC₅₀<1 μM, and “B” indicates IC₅₀≥1 μM.

TABLE 7
The results of the activity of inhibiting phosphorylation of KRAS
G12C protein-mediated downstream signaling (p-ERK)

| No. | IC₅₀ |
|---|---|
| Compound 1 | A |
| Compound 2 | A |
| Compound 3 | A |
| Compound 4 | A |
| Compound 5 | A |
| Compound 6 | A |
| Compound 7 | A |
| Compound 8 | A |
| Compound 9 | A |
| Compound 10 | A |
| Compound 11 | A |
| Compound 12 | A |
| Compound 13 | A |
| Compound 14 | A |
| Compound 15 | A |
| Compound 16 | A |
| Compound 17 | A |
| Compound 18 | A |
| Compound 19 | A |
| Compound 20 | A |
| Compound 21 | A |
| Compound 22 | A |
| Compound 23 | A |
| Compound 24 | A |
| Compound 25 | A |
| Compound 26 | A |
| Compound 27 | A |
| Compound 28 | A |
| Compound 29 | A |
| Compound 30 | A |
| Compound 31 | A |
| Compound 32 | A |
| Compound 33 | A |
| Compound 34 | A |
| Compound 35 | A |
| Compound 36 | A |
| Compound 37 | A |
| Compound 38 | A |
| Compound 39 | A |
| Compound 40 | A |
| Compound 41 | A |
| Compound 42 | A |

As can be seen from the data in the above table, the compounds of the present invention can effectively inhibit KRAS G12C protein-mediated phosphorylation of H358 cell downstream signal (p-ERK), and can be used as KRAS G12C protein inhibitors.

### Example 8: Cell Proliferation Inhibition Tests by In Vitro 3D Culture of Tumor Cells

In order to examine the antitumor activity of the compounds of the present invention, compounds 1, 17, 18, 41 and 42 were used as representative compounds to test for their activity of inhibiting proliferation of 3 KRAS G12C mutant tumor cells (H358, H1373 and MIA PaCa-2); meanwhile, given the fact that the K-RAS wild type plays an important role in normal cell physiological functions and inhibiting the K-RAS wild type may result in severe toxic side effects, the K-RAS wild type PC-9 cells were adopted to evaluate the selectivity of the test compounds for the wild type so as to obtain compounds with better activity, higher selectivity and better safety.

### (1) Reagent, Consumable and Device Information:

| | Supplier | Batch No. |
|---|---|---|
| Reagent | | |
| PC-9 cell line (K-RAS wild type) | ATCC | CRL-5807 |
| H358 cell line (K-RAS G12C) | ECACC | 90071810 |
| H1373 cell line (K-RAS G12C) | ATCC | CRL-5866 |
| MIA PaCa-2 cell line (K-RAS G12C) | ATCC | CRL-1420 |
| CellTiter-Glo ® 3D cell viability assay kit | Promega | G9683 |
| RPMI 1640 medium | Gibco | A10491-01 |
| DMEM medium | Gibco | 11995-065 |
| TransSerum EQ fetal bovine serum | Transgene | FS201-02 |
| (500 mL) | | |
| Penicillin-streptomycin | Gibco | 15140-122 |
| Consumables | | |
| T75 cell culture flask | Corning | 430641 |
| 384-well round plate | Corning | 3657 |
| 384-well polypropylene microwell | LABCYTE | PP-0200 |
| plate 2.0 meeting Echo requirement | | |
| with transparent bottom | | |
| Device | | |
| Echo 550 liquid processor | Labcyte | Echo 550 |
| Plate reader | Perkin | Envision |
| | Elmer | 2104 |
| Centrifuge | Eppendorf | 5810R |
| Shaker | VWR | 12620-928 |
| Countess ™ II automatic cell counter | Invitrogen | AMQAX1000 |

### (2) Cell Culture:

a) On day 1, seed cells were placed in T75 a flask.
b) On day 3, the medium was taken out and washed once with DPBS.
c) Cells were trypsinized with 2 mL TrypLE™ Express enzyme at room temperature (RT) or 37° C. until cells detached.
d) 5 mL of fresh medium was added to suspend the cells, and then the suspension was centrifuged at 1000 rpm and room temperature for 5 min.
e) The supernatant was discarded, and the cells were resuspended in 5 mL of fresh medium and counted using Countess™ H.
f) The cells were seeded back into the T75 flask for further culture or placed into an assay plate for 3D cell proliferation assay.

### (3) 3D Cell Proliferation Assay:

a) On day 1, 200 nL of a diluted compound (for proliferation of PC-9 cells of K-RAS wild type, test compounds were serially diluted 3-fold from a concentration of 50 μM; for K-RAS-G12C mutations, test compounds were serially diluted 3-fold from a concentration of 1 μM) was added to each hole. The cells were seeded at a density of 600 cells/well in a 384-well plate with 40 μL of medium per well and a final DMSO concentration of 0.5%.
b) On day 4, a 3D CTG reagent was added to each well, followed by shaking at room temperature for 1 h.
c) The signals were recorded using Envision.

### (4) Data Analysis:

a) Robustness was examined using 0.5% DMSO and data of blank control medium:
H=mean (DMSO); L=mean (medium);
SD (H)=STDEV (DMSO); SD (L)=STDEV (medium);

### CV % (DMSO)=100×(SD DMSO/mean DMSO);

CV % (medium)=100×(SD medium/mean medium);
S/B=mean DMSO/mean medium;
Z′=1-3×(SD DMSO+SD medium)/(mean DMSO−mean medium);
Cell viability inhibition (%)=(mean_H−sample)/(mean_H−mean_L)×100%;
b) Cpd IC50 was fitted according to a nonlinear regression equation:

Y=peak valley value+(peak top value−peak valley value)/(1+10\hat{ }((Log IC50−X)×HillSlope));

X: logarithm of compound concentration;
Y: percent inhibition (% inhibition);
Peak top and peak valley values: the plateau period units are the same as Y;
log IC50: the same logarithmic unit as X;
HillSlope: slope coefficient or Hill slope.

### (5) Results:

The related results are shown in Tables 8 through 10.

TABLE 8
Inhibition of H358 tumor cell proliferation
and selectivity for wild type by test compounds

| | PC-9 (K-RAS | H358 (K-RAS | Selectivity |
|---|---|---|---|
| Compound | wild type) | G12C) | (fold number of |
| No. | IC₅₀ (μM) | IC₅₀ (nM) | mutant vs. wild type) |
| AMG510 | 17.5 | 6.41 | 2730 |
| MRTX849 | 2.28 | 2.68 | 851 |
| Compound 1 | 4.81 | 0.55 | 8745 |
| Compound 17 | 4.69 | 0.34 | 13794 |
| Compound 18 | 1.94 | 2.26 | 858 |
| Compound 41 | 2.31 | 0.28 | 8250 |
| Compound 42 | 2.37 | 0.62 | 3823 |

TABLE 9
Inhibition of H1373 tumor cell proliferation
and selectivity for wild type by test compounds

| | PC-9 (K-RAS | H1373 (K- | Selectivity |
|---|---|---|---|
| Compound | wild type) | RAS G12C) | (fold number of |
| No. | IC₅₀ (μM) | IC₅₀ (nM) | mutant vs. wild type) |
| AMG510 | 17.5 | 3.95 | 4430 |
| MRTX849 | 2.28 | 5.52 | 413 |
| Compound 1 | 4.81 | 0.68 | 7074 |
| Compound 17 | 4.69 | 0.32 | 14656 |
| Compound 41 | 2.31 | 0.18 | 12833 |
| Compound 42 | 2.37 | 0.26 | 9115 |

TABLE 10
Inhibition of MIA PaCa-2 tumor cell proliferation and
selectivity for wild type by test compounds

| | PC-9 (K-RAS | MIA PaCa-2 | Selectivity |
|---|---|---|---|
| Compound | wild type) | (K-RAS G12C) | (fold number of |
| No. | IC₅₀ (μM) | IC₅₀ (nM) | mutant vs. wild type) |
| AMG510 | 17.5 | 10.56 | 1657 |
| MRTX849 | 2.28 | 3.21 | 710 |
| Compound 1 | 4.81 | 0.49 | 9816 |
| Compound 17 | 4.69 | 0.44 | 10659 |
| Compound 18 | 1.94 | 2.40 | 808 |
| Compound 41 | 2.31 | 0.31 | 7452 |
| Compound 42 | 2.37 | 0.51 | 4647 |

As can be seen from the above data, the activity of the compounds of the present invention in inhibiting K-RAS G12C protein-mediated proliferation of various tumor cells is significantly better than that of non-deuterated reference compounds AMG510 (CAS: 2252403-56-6) and MRTX849 (CAS: 2326521-71-3); and the compounds of the present invention have superior selectivity for K-RAS wild type cells over the non-deuterated reference compounds, with lower expected potential risk of side effects due to K-RAS wild type inhibition than that of the reference compounds.

### Example 9: Permeability and Efflux Caco-2 Assays

### (1) Reagent, Consumable and Device Information:

| | Supplier |
|---|---|
| Reagent | |
| Caco-2 cells | ATCC |
| HEPES | Thermo Fisher |
| Hanks' balanced salt solution (HBSS) | Thermo Fisher |
| Non-essential amino acids (NEAA) | Thermo Fisher |
| Penicillin | Solarbio |
| Streptomycin | Solarbio |
| Trypsin/EDTA | Solarbio |
| Fetal bovine serum (FBS) | Corning |
| DMEM medium | Corning |
| Consumables | |
| HTS-96-well Transwell plate | Corning |
| Device | Supplier |
| Millicell resistance measurement system | Millipore |
| Cellometer ® Vision cell analyzer | Nexcelom Bioscience |
| Infinite 200 PRO microplate reader | Tecan |
| Triple Quad 5500/Triple Quad 4500 | AB Sciex |
| mass spectrometer | |
| LC-30A liquid phase system | Shimadzu |

### (2) Test Method:

Preparation of stock solution: A) preparation of 1 L HBSS (25 mM HEPES, pH 7.4): 5.958 g of HEPES and 0.35 g of sodium bicarbonate were weighed and dissolved into 900 mL of purified water, and then 100 mL of 10×HBSS was added; the resulting mixture was stirred to be well mixed, adjusted to pH 7.4, and filtered, and the desired solution was thus obtained; B) preparation of test solutions of test compounds and a control drug: a high-concentration DMSO stock solution of a test compound and control drugs (digoxin and metoprolol) were first prepared and diluted with DMSO to 2 mM stock solutions, which were then correspondingly diluted with HBSS (25 mM HEPES, pH 7.4) to obtain test solutions with a concentration of 10 μM.

Drug penetration test: the Transwell culture plate was retrieved from the incubator. Cell monolayer membranes were rinsed twice with HBSS (25 mM HEPES, pH 7.4), and the cells were incubated at 37° C. for 30 min; 75 μL of donor end solution was added to each well of the upper chamber (top end) and 235 μL of acceptor end solution was added to each well of the lower chamber (base end) so as to determine the transport rate of the test compound from the top end to the base end; 75 μL of acceptor end solution was added to each well of the upper chamber (top end) and 235 μL of donor end solution was added to each well of the lower chamber (base end) so as to determine the transport rate of the test compound from the base end to the top end; after combination of the upper and lower transport device, incubation was performed at 37° C. for 2 h; 50 μL of samples were transferred from the working solution preparation plate to 200 μL of acetonitrile containing an internal standard to serve as 0-minute dosing samples for detection; after the completion of the incubation, 50 μL of sample were taken from each hole of the upper and lower chambers of the Transwell culture plate to new sample tubes; 200 μL of acetonitrile containing an internal standard was added to the sample tubes, which were then vortexed for 10 mM, followed by centrifugation at 3220 g for 30 mM; 150 μL of the supernatants were pipetted and diluted with an equal volume of water for LC-MS/MS analysis. All samples were prepared in double parallel. The integrity of the cell monolayer membrane after 2 hours of incubation was assessed using leakage of fluorescein; fluorescein stock solution was diluted with HBSS (25 mM HEPES, pH 7.4) to a final concentration of 100 μM. 100 μL of fluorescein solution was added to each well of the upper Transwell plate and 300 μL of HBSS (25 mM HEPES, pH 7.4) was added to each well of the lower receiving plate; after incubation at 37° C. for 30 min, 80 μL of the solution was pipetted from the upper and lower layers of each well to a new 96-well plate. Fluorescence measurement was performed using a microplate reader at an excitation wavelength of 485 nm and an emission wavelength of 530 nm.

### (3) Data Analysis:

Calculations were performed based on the data using Microsoft Excel software.

The apparent permeability coefficients (Pₐₚₚ, unit: cm/s) of the compounds in Caco-2 cells were calculated from the specific concentrations at the acceptor and donor ends, and the specific calculation formula is as follows:

$${P}_{app} = \frac{{V}_{A}}{Area \times time} \times \frac{{[ drug ]}_{acceptor}}{{[ drug ]}_{initial , donor}}$$

where V_{A} represents the volume of the acceptor end solution (0.3 mL for Ap→Bl, 0.1 mL for Bl→Ap); Area represents the area of the Transwell-96 well plate (0.143 cm²); time represents the incubation time (unit: s); [drug]_{acceptor} represents the drug concentration (ratio of peak area to internal standard area) at the acceptor end; [drug]_{initial, donor} represents the drug concentration at the donor end (ratio of peak area to internal standard area).

The efflux ratio was calculated as follows:

$$Efflux Ratio = \frac{{P}_{app ( B - A )}}{{P}_{app ( A - B )}}$$

where P_{app (B-A)} represents the apparent permeability coefficient from base end to top end; P_{app (A-B)} represents the apparent permeability coefficient from top end to base end.

### (4) Results:

The related results are shown in Table 11.

TABLE 11
Permeability and efflux ratios of the test compounds

| | P_{app (A-B)} | P_{app (B-A)} | Efflux |
|---|---|---|---|
| Compound No. | (10⁻⁶, cm/s) | (10⁻⁶, cm/s) | ratio |
| Metoprolol | 22.64 | 19.49 | 0.86 |
| Digoxin | 0.25 | 16.41 | 65.22 |
| AMG510 | 0.60 | 27.41 | 45.85 |
| MRTX-1257 | 0.46 | 7.50 | 16.31 |
| Compound 1 | 0.63 | 5.50 | 8.70 |

From the above data, it can be seen that the compounds of the present invention have significantly improved permeability or efflux ratios relative to the non-deuterated reference compound, MRTX-1257 or AMG-510.

### Example 10: Stability Test of Test Compounds in Human Plasma

To examine the stability of the test compounds in plasma, representative compounds were selected for evaluation.

### (1) Reagent Information:

| Species | Strain | Gender | Supplier |
|---|---|---|---|
| Human | N/A | Mix | Corning |

### (2) Test Method:

Preparation of stock solution: a test compound was dissolved in DMSO to prepare a 1 mM stock solution for later use; Propantheline Bromide was dissolved in acetonitrile to prepare a 1 mM stock solution as a positive control for later use.

398 μL of human plasma was added to each well of an incubation plate, which was then pre-incubated at 37° C. for 15 min. To each well was added 2 μL of the stock solution of the test compound and the stock solution of the positive control such that the final concentrations were 5 KM and the concentration of the organic solvent was 0.5%. Each compound was prepared in duplicate, followed by incubation at 37° C. 50 μL of samples were taken from the reaction samples in equal amounts at 0, 30, 60, 120, 180 and 240 minutes, and 450 μL of cold acetonitrile containing an internal standard was added to terminate the reaction. All samples were vortexed for 10 mM, followed by centrifugation at 3220 g for 30 mM to precipitate proteins. 100 μL of the supernatants were transferred to a new plate, dilute with ultra-pure water, and analyzed by LC-MS/MS.

### (3) Data Analysis:

Calculations were performed based on the data using Microsoft Excel software. Data results were calculated according to peak area ratios.

The percentage of remaining compound for each time point was calculated as follows:

Remaining percentageₘᵢₙ(%)=peak area ratiosₘᵢₙ/peak area ratioₘᵢₙ×100%

where peak area ratios represents the peak area ratio of the test compound to the internal standard compound at t min; peak area ratioₘᵢₙ represents the peak area ratio of the test compound to the internal standard compound at 0 min (initial).

The slope value (k) was determined by natural logarithm linear regression of the curve of the remaining percentage of the drug over incubation time.

In vitro t_{1/2} was calculated based on the slope value, and the specific calculation formula is as follows: t_{1/2}=0.693/k.

### (4) Results:

The related results are shown in Table 12.

TABLE 12
Stability of the test compounds in human plasma

| | Remaining percentage (%) | | | | | | t_{1/2} |
|---|---|---|---|---|---|---|---|
| Compound No. | 0 min | 30 min | 60 min | 120 min | 180 min | 240 min | (min) |
| Propantheline Bromide | 100.00 | 33.35 | 7.71 | 0.15 | BLOD | BLOD | 19.02 |
| MRTX-1257 | 100.00 | 88.91 | 82.31 | 55.64 | 32.06 | 18.12 | 96.47 |
| Compound 1 | 100.00 | 92.80 | 76.27 | 58.94 | 39.27 | 25.75 | 121.92 |
| Compound 17 | 100.00 | 97.00 | 92.97 | 78.91 | 59.98 | 47.82 | 218.00 |
| Compound 18 | 100.00 | 90.96 | 95.39 | 97.36 | 92.57 | 89.93 | ∞ |
| Compound 41 | 100.00 | 96.01 | 75.85 | 56.99 | 34.80 | 26.83 | 118.68 |
| Compound 42 | 100.00 | 90.36 | 77.31 | 57.19 | 36.19 | 27.49 | 123.40 |

From the above data, it can be seen that the compounds of the present invention have significantly improved metabolic stability in human plasma and significantly extended half-life relative to the non-deuterated reference compound MRTX-1257.

### Example 11: Tests for Metabolic Stability of Test Compounds in Liver Microsomes

To examine the in vitro metabolic stability of the test compounds in liver microsomes of different species, representative compounds were selected, and their stability was evaluated by measuring the concentration of the test compounds in the incubation system using LC/MS/MS and calculating their intrinsic clearance in the microsome system.

### (1) Reagent Information:

The test concentrations for test compounds, reference compound MRTX-1257 and control compound verapamil were all 1 μM. The method is specifically as follows: each compound was first dissolved in DMSO to prepare a 200 μM working solution and added to the system solution at the time of testing to form a solution with a final concentration of 1 μM.

The microsomes were stored in a −80° C. freezer. The specific information is shown in the table below.

| Species | Strain | Gender | Supplier |
|---|---|---|---|
| Human | N/A | Mix | Corning |
| Mouse | ICR (CD-1) mouse | Male | Corning |

Preparation of working solutions of compounds: a test compound and the control drug verapamil were dissolved in DMSO to prepare high-concentration stock solutions, which were diluted with DMSO into 200 μM working solution before use, with the final concentrations of the test compound and verapamil at 1 μM.

Preparation of phosphate-buffered saline (100 mM, pH 7.4): 7.098 g of disodium phosphate was weighed and ultrasonically dissolved in 500 mL of purified water to obtain a solution A; 3.400 g of monopotassium phosphate was weighed and ultrasonically dissolved in 250 mL of purified water to obtain a solution B; solution B was added to solution A until the pH value is 7.4, and the desired solution was thus obtained.

Preparation of NADPH solution (10 mM): a proper amount of NADPH was weighed and dissolved in phosphate-buffered saline to prepare a working solution with a concentration of 10 mM, and the desired solution was thus obtained.

Preparation of incubation system: an incubation system was prepared as described in the table below. The incubation system was always preheated in a 37° C. water bath for 15 min before use

| | Stock solution | | Final concentration |
|---|---|---|---|
| Component | concentration | Volume | of the system |
| Microsome | 20 mg/mL | 6.25 μL | 0.5 mg/mL |
| Phosphate- | 100 mM | 216.25 μL | 100 mM |
| buffered salt | | | |

### (2) Test Method:

25 μL, of NADPH or phosphate buffered saline was transferred to the incubation system described above, and 2 μL, of 200 μM of the test compound or verapamil was added. The sample with NADPH added was prepared in duplicate; the NADPH negative sample was prepared in single parallel. 30 μL, of suspension was taken at 0.5, 5, 15, 30 and 60 minutes. The reaction system was terminated by adding 180 μL, of acetonitrile containing an internal standard and vortexed for 10 min. Then, the plate was centrifuged at 3220 g for 20 min for protein precipitation. The plate was left in a refrigerator at 4° C. for 30 min and then centrifuged again at 3220 g for 20 min. 100 μL, of the supernatant was transferred to a feeding plate, and 100 μL, of purified water was added. The mixture was well mixed for UPLC-MS/MS analysis.

### (3) Data Analysis:

Calculations were performed based on the data using Microsoft Excel software. The results based on the data were obtained by calculation according to the peak area ratio, and the peak area was detected through an extracted ion chromatogram.

The slope value (k) was determined by linear fitting of the natural logarithm of drug elimination percentage over time.

The in vitro half-life (t_{1/2}) was calculated based on the slope value using the following formula: in vitro t_{1/2}=0.693/k.

The in vitro clearance rate (CLᵢₙ, unit: μL/min/mg) was calculated as follows: In vitro CLᵢₙₜ=kV/N

where V represents the incubation volume per well (400 μL); n represents the microsome content per well (0.2 mg).

### (4) Results:

The related results are shown in Table 13.

TABLE 13
Stability of the test compounds in liver microsomes

| Compound | | t_{1/2} | CLᵢₙₜ (μL/min/ |
|---|---|---|---|
| No. | Species | (min) | mg protein) |
| Verapamil | Human | 5.36 | 258.54 |
| | Mouse | 5.11 | 271.57 |
| MRTX-849 | Human | 19.76 | 70.17 |
| | Mouse | 10.00 | 138.56 |
| MRTX-1257 | Human | 25.58 | 54.30 |
| | Mouse | 18.49 | 74.97 |
| Compound 1 | Human | 29.28 | 47.34 |
| | Mouse | 20.85 | 66.51 |
| Compound 18 | Human | 19.26 | 71.97 |
| | Mouse | 12.27 | 113.08 |
| Compound 41 | Human | 26.02 | 53.35 |
| | Mouse | 14.56 | 95.34 |
| Compound 42 | Human | 27.17 | 51.02 |
| | Mouse | 22.78 | 60.87 |

As can be seen from the above table data, the compounds of the present invention have increased metabolic stability in liver microsomes, decreased metabolic rate, and prolonged elimination half-life relative to the non-deuterated reference compound MRTX-1257, particularly to MRTX-849.

### Experimental Example 12: In Vivo Pharmacodynamic Test Using Xenograft Tumor Nude Mouse Model of NCI-H358 Cells

To evaluate the in vivo antitumor activity of the compounds of the present invention, in vivo efficacy was evaluated using nude mouse subcutaneous xenograft tumor model of NCI-H358 cells (human non-small cell lung cancer cells) with K-RAS G12C mutation.

### (1) Consumable and Device Information:

| Consumables | Supplier (Brand) |
|---|---|
| RPMI1640 | Invitrogen Trading (Shanghai) Co., Ltd. (GIBCO) |
| FBS | GE Healthcare (Shanghai) Co., Ltd. (Hyclone) |
| PBS | GE Healthcare (Shanghai) Co., Ltd. (Hyclone) |
| Pancreatin | Invitrogen Trading (Shanghai) Co., Ltd. (GIBCO) |
| Antibiotics | Invitrogen Trading (Shanghai) Co., Ltd. (GIBCO) |
| solutol | Shanghai Xietai Chem Co., Ltd. |
| T150 culture flask | Corning |
| 50 mL centrifuge tube | Shanghai J&W Industry Co., Ltd. |
| 10 mL pipette | Corning |
| Syringe | Jiangsu Greatwall Medical Devices Co., Ltd. |
| Dispensing tube | Worldwide Glass Resource |
| balb/c nude mouse | Shanghai Sippe-Bk Lab Animal Co., Ltd. |
| Device | Supplier |
| Analytical balance | Sartorius |
| Ordinary balance | Changzhou Tianzhiping Instruments Co., Ltd. |
| Vernier caliper | Mitutoyo |
| Biosafety cabinet | AIRTECH |
| Centrifuge | Thermo |

### (2) Experimental Method:

1) Cell culture: NCI-H358 cells (ECACC, Cat. No. 95111733) were cultured in vitro in monolayer in RPMI1640 medium supplemented with 10% FBS, 100 U/mL penicillin and 100 μg/mL streptomycin at 37° C. in a 5% CO₂ incubator. The cells were digested with trypsin-EDTA twice a week for passaging as per conventional practice. When the cell saturation reached 80%-90% and a required amount was achieved, the cells were collected and counted for later use.
2) Sample preparation: a certain amount of the compound (converted according to the purity) was precisely weighed, and 0.450 mL of DMSO solution was added; the resulting mixture was fully vortexed, and 0.450 mL of Solutol was added; the mixture was vortexed for a moment, and 8.100 mL of water was added; after vortexing, a clear solution was obtained, with the final concentration of each compound being 1.5 mg/mL. 5% DMSO/5% Solutol/90% water was used as a blank control.
3) Tumor inoculation and drug administration: BALB/c nude mice, female, 6-8 weeks old, weighing 18-20 g. The experiment started after 3-7 days of adaptive feeding. 0.1 mL of NCI-H358 cells (5×10⁶ cells) were inoculated subcutaneously on the right back of each mouse. The mice were grouped for administration when the average tumor volume reached approximately 150 mm³. The animals were weighed before administration and the tumor volume was measured. Grouping was randomly performed according to the tumor volume. The experimental groups and administration regimens are shown in Table 14.

TABLE 14
Animal groups and administration regimens

| | Each | | | | | |
|---|---|---|---|---|---|---|
| | group | | | Dosing | | |
| | Sample | | Dosage | volume | Route of | Frequency of |
| Groups | size | Compound | (mg/kg) | (μL/g) | administration | administration |
| 1 | 8 | Solvent | — | 10 | PO | QD |
| | | control | | | | |
| 2 | 8 | MRTX-849 | 15 | 10 | PO | QD |
| 3 | 8 | Compound 1 | 15 | 10 | PO | QD |
| 4 | 8 | Compound 17 | 15 | 10 | PO | QD |
| 5 | 8 | Compound 41 | 15 | 10 | PO | QD |
| 6 | 8 | Compound 42 | 15 | 10 | PO | QD |

Note:
the administration volume was determined to be 10 μL/g according to the body weight of the mice. If body weight decreases by more than 15%, the administration regimen should be adjusted accordingly.

### 4) Gross Observations and Calculation of Anti-Tumor Activity:

Animals were monitored daily for health and death, and routine examinations include observation of the effect of tumor growth and drug treatment on the daily performance of the animals, such as behavioral activities, food and water intake, weight changes (measured once every other day), appearance, or other abnormal conditions. Animal mortality and side effects within each group were recorded based on the number of animals in the group.

Tumor diameters were measured 2-3 times weekly using a vernier caliper.

The tumor volume was calculated as follows:

V=0.5a×b²

where a and b represent the long and short diameters of the tumor, respectively.

The anti-tumor therapeutic effect of the compound was evaluated by tumor growth inhibition rate TGI (%) or relative tumor proliferation rate T/C (%).

TGI (%) was calculated as follows:

TGI (%)=[(1−(average tumor volume at the end of administration in a treatment group−average tumor volume at the start of administration of the treatment group))/(average tumor volume at the end of treatment of the solvent control group−average tumor volume at the start of treatment of the solvent control group)]×100%.

T/C (%) was calculated as follows:

T/C (%)=T_{RTV}/C_{RTV}×100%

where T_{RTV} represents the relative tumor volume of the treatment group; C_{RTV} represents the relative tumor volume of the solvent control group.

Based on the results of tumor measurement, the relative tumor volume (RTV) was calculated as follows:

RTV=Vₜ/V₀

where V₀ represents the average tumor volume measured at the time of grouping and administration (i.e., d0), Vₜ represents the average tumor volume in a certain measurement, and T_{RTV} and C_{RTV} take the data on the same day.

After the completion of the experiment, tumors were weighed, and the percentage of T_{weight}/C_{weight} was calculated, where T_{weight} and C_{weight} represent the tumor weights of the treatment group and the solvent control group, respectively.

Data calculation and analysis: T test was employed for comparison between two groups. One-way ANOVA was employed for comparison between three or more groups. If there is a significant difference in F-values, multiple comparisons should be made after ANOVA analysis. All data analyses were performed with SPSS 17.0. p<0.05 was defined as a significant difference.

### 5) Summary Results:

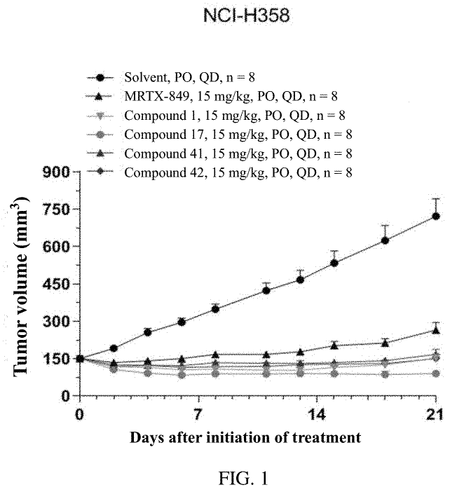

The related results are shown in Table 15 and FIG. 1.

TABLE 15
The results of the inhibition of the growth of NCI-
H358 tumor in nude mice by the test compounds

| | Day 0 | | Day 21 | | Day 21 | |
|---|---|---|---|---|---|---|
| | Tumor | Body | Tumor | Body | Tumor | |
| | volume | weight | volume | weight | inhibition | |
| | (mm³) | (g) | (mm³) | (g) | rate | |
| | V_{average} ± | W_{average} ± | V_{average} ± | W_{average} ± | TGI | Tumor |
| Groups | SEM | SEM | SEM | SEM | (%) | regression |
| Solvent | 151 ± 9 | 22.5 ± 0.6 | 722 ± 70 | 24.0 ± 0.9 | | 0/8 |
| control | | | | | | |
| group | | | | | | |
| MRTX-849 | 151 ± 9 | 22.8 ± 0.6 | 265 ± 30 | 23.7 ± 0.5 | 80.0% | 0/8 |
| Compound 1 | 150 ± 10 | 22.5 ± 0.5 | 153 ± 14 | 23.4 ± 0.5 | 99.5% | 3/8 |
| Compound 17 | 151 ± 10 | 22.6 ± 0.7 | 90 ± 12 | 23.7 ± 0.8 | 110.7% | 8/8 |
| Compound 41 | 151 ± 8 | 22.7 ± 0.4 | 168 ± 20 | 23.4 ± 0.6 | 97.0% | 2/8 |
| Compound 42 | 150 ± 9 | 22.8 ± 0.6 | 151 ± 18 | 23.8 ± 0.6 | 99.8% | 4/8 |

As can be seen from the in vivo efficacy results, the compounds of the present invention have significantly better effect on inhibiting the growth of NCI-H358 tumor than the control compound MRTX-849 at the same dosage; the tumors regressed in some of the treatment groups, especially in the compound 17 group, in which the tumors in all of the animals regressed. There was no significant change in body weight in the treatment groups.

What is claimed is:

1. A compound of formula I: 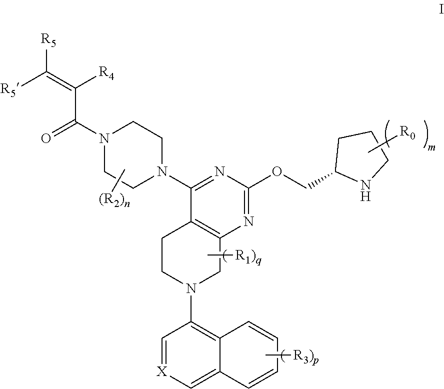
or a pharmaceutically acceptable salt, a solvate, a hydrate, a stereoisomer, a tautomer, an isotopically labeled compound or a prodrug thereof or a mixture thereof in any proportion, wherein,
X is —CR₆═ or —N═;
each R₀ is independently hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C, preferably alkyl, cycloalkyl, methyl-d₃, ethyl-d₅, ethyl-2,2,2-d₃, isopropyl-d₇, methyl-¹⁴C, methyl-¹³C or methyl-¹¹C, and more preferably alkyl, methyl-d₃, ethyl-d₅, ethyl-2,2,2-d₃, isopropyl-d₇, methyl-¹⁴C, methyl-¹³C or methyl-¹¹C;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen; and the hydrogen in the R₁ structure is optionally substituted with 0 or more R₇;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl;
each R₃ is independently hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C, preferably halogen, alkyl, methyl-d₃, ethyl-d₅, ethyl-2,2,2-d₃, isopropyl-d₇, methyl-¹⁴C, methyl-¹³C, or methyl-¹¹C, and more preferably halogen, alkyl, methyl-d₃, methyl-¹⁴C, methyl-¹³C, or methyl-¹¹C, and the hydrogen in the R₃ structure is optionally substituted with 0 or more R₇;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl, and the hydrogen in the R₄, R₅ and R_{5′} structures is optionally substituted with 1 or more substituents, wherein each of the substituents is independently deuterium, halogen, amino, hydroxy, alkoxy, alkanoyl, alkanoyloxy, alkoxycarbonyl, alkylsulfonamido, or cyano;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
each R₇ is independently hydrogen, deuterium, halogen, alkyl, cycloalkyl, cyano, amino, haloalkyl or haloalkoxy, preferably hydrogen, deuterium, halogen, cyano or amino, and more preferably deuterium, halogen or cyano;
m, n, p and q are each independently 0, 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

2. The compound according to claim 1, which is a compound of formula I-A, 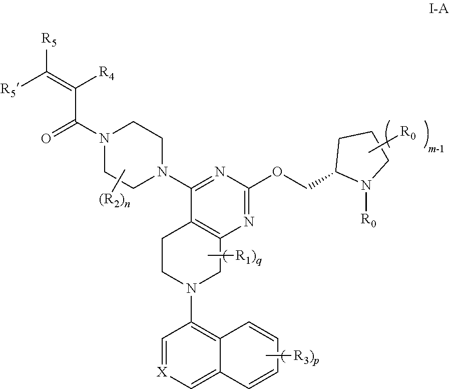
wherein,
X is —CR₆═ or —N═;
on the pyrrolidine ring, R₀ linked to the nitrogen atom is methyl-d₃, ethyl-d₅, ethyl-2,2,2-d₃, isopropyl-d₇, methyl-¹⁴C, methyl-¹³C or methyl-¹¹C; the remaining R₀ is hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₀ is hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, heteroalkyl, alkenyl, alkynyl, cycloalkyl, heterocycloalkyl, cyano, amino, haloalkyl or haloalkoxy, preferably hydrogen, deuterium, halogen, alkyl, cycloalkyl, cyano, amino, haloalkyl or haloalkoxy;
each R₂ is independently hydrogen, deuterium, halogen, alkyl, alkenyl, alkynyl, cycloalkyl, cyano, alkoxy, haloalkyl or haloalkoxy, preferably hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy;
each R₃ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, preferably hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C, and more preferably halogen, alkyl, methyl-d₃, methyl-¹⁴C, methyl-¹³C, or methyl-¹¹C;
R₄, R₅ and R_{5′} are each independently hydrogen, deuterium, halogen, alkyl, alkenyl, alkynyl, cycloalkyl, heterocycloalkyl, alkylaminoalkyl, cycloalkylaminoalkyl, amino, hydroxy or haloalkyl, preferably hydrogen, deuterium, halogen, alkyl, cycloalkyl, alkylaminoalkyl, cycloalkylaminoalkyl, amino or haloalkyl;
R₆ is hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl or haloalkoxy, preferably hydrogen, deuterium, halogen, alkyl, cycloalkyl, cyano, amino, haloalkyl or haloalkoxy;
m is 1 or 2, and n, p and q are each independently 0, 1 or 2.

3. The compound according to claim 1, which is a compound of formula I-A, 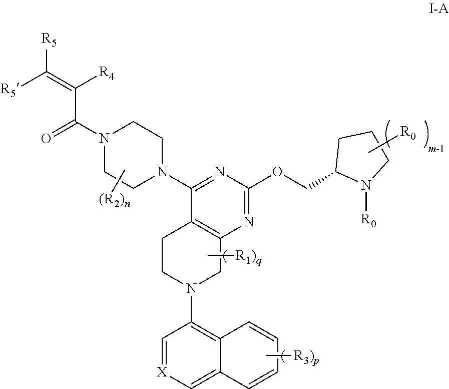
wherein,
X is —CR₆═ or —N═;
on the pyrrolidine ring, R₀ linked to the nitrogen atom is alkyl, methyl-d₃, ethyl-d₅, ethyl-2,2,2-d₃, isopropyl-d₇, methyl-¹⁴C, methyl-¹³C or methyl-¹¹C; the remaining R₀ is hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₀ is hydrogen, alkyl, cycloalkyl, methyl-d₃, ethyl-d₅, ethyl-2,2,2-d₃, isopropyl-d₇, methyl-¹⁴C, methyl-¹³C or methyl-¹¹C; more preferably, the remaining R₀ is hydrogen, alkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl;
each R₃ is independently hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C, preferably halogen, alkyl, methyl-d₃, ethyl-d₅, ethyl-2,2,2-d₃, isopropyl-d₇, methyl-¹⁴C, methyl-¹³C, or methyl-¹¹C, and more preferably halogen, alkyl, methyl-d₃, methyl-¹⁴C, methyl-¹³C, or methyl-¹¹C;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
m is 1 or 2, and n, p and q are each independently 0, 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

4. The compound according to claim 1, which is a compound of formula I-B, 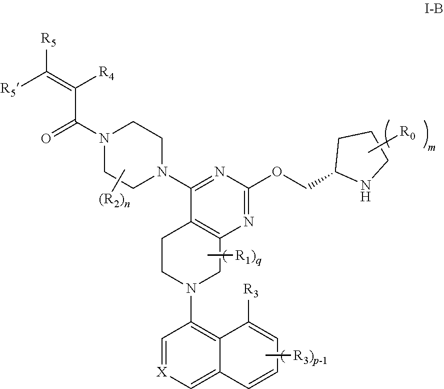
wherein,
X is —CR₆═ or —N═;
each R₀ is independently hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, preferably hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C, or methyl-¹¹C, and more preferably alkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, heteroalkyl, alkenyl, alkynyl, cycloalkyl, heterocycloalkyl, cyano, amino, haloalkyl or haloalkoxy, preferably hydrogen, deuterium, halogen, alkyl, cycloalkyl, cyano, amino, haloalkyl or haloalkoxy;
each R₂ is independently hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl or haloalkoxy, preferably hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy;
on the aromatic ring containing X, R₃, together with the tetrahydropyridopyrimidine ring, present in the 1,8-disubstituted form is methyl-d₃, ethyl-d₅, ethyl-2,2,2-d₃, isopropyl-d₇, methyl-¹⁴C, methyl-¹³C or methyl-¹¹C; the remaining R₃ is hydrogen, deuterium, halogen, alkyl, heteroalkyl, alkenyl, alkynyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₃ is hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C, or methyl-¹¹C;
R₄, R₅ and R_{5′} are each independently hydrogen, deuterium, halogen, alkyl, alkenyl, alkynyl, cycloalkyl, heterocycloalkyl, alkylaminoalkyl, cycloalkylaminoalkyl, amino, hydroxy or haloalkyl, preferably hydrogen, deuterium, halogen, alkyl, cycloalkyl, alkylaminoalkyl, cycloalkylaminoalkyl, amino or haloalkyl;
R₆ is hydrogen, deuterium, halogen, alkyl, alkenyl, alkynyl, cycloalkyl, cyano, cyanoalkyl, alkoxyalkyl, alkylaminoalkyl, acyl, substituted acyl, sulfonyl, amino, alkylamino, hydroxy, alkoxy, haloalkyl or haloalkoxy, preferably hydrogen, deuterium, halogen, alkyl, cycloalkyl, cyano, amino, haloalkyl or haloalkoxy;
m, n and q are each independently 0, 1 or 2, and p is 1 or 2.

5. The compound according to claim 1, which is a compound of formula I-B, 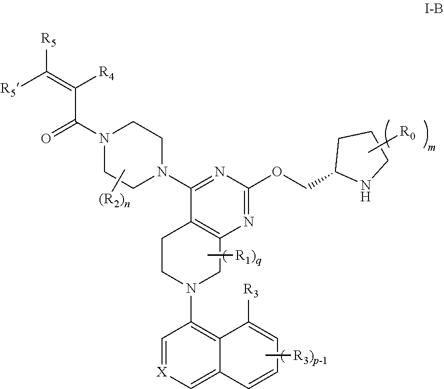
wherein,
X is —CR₆═ or —N═;
each R₀ is independently hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C, preferably alkyl, cycloalkyl, methyl-d₃, ethyl-d₅, ethyl-2,2,2-d₃, isopropyl-d₇, methyl-¹⁴C, methyl-¹³C or methyl-¹¹C, and more preferably alkyl, methyl-d₃, ethyl-d₅, ethyl-2,2,2-d₃, isopropyl-d₇, methyl-¹⁴C, methyl-¹³C or methyl-¹¹C;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl;
on the aromatic ring containing X, R₃, together with the tetrahydropyridopyrimidine ring, in the 1,8-disubstituted form is halogen, alkyl, methyl-d₃, ethyl-d₅, ethyl-2,2,2-d₃, isopropyl-d₇, methyl-¹⁴C, methyl-¹³C or methyl-¹¹C; the remaining R₃ is hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₃ is hydrogen, halogen, alkyl, methyl-d₃, ethyl-d₅, ethyl-2,2,2-d₃, isopropyl-d₇, methyl-¹⁴C, methyl-¹³C or methyl-¹¹C; more preferably, the remaining R₃ is hydrogen, halogen, alkyl, methyl-d₃, methyl-¹⁴C, methyl-¹³C or methyl-¹¹C;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
m, n and q are each independently 0, 1 or 2, and p is 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

6. The compound according to claim 1, which is a compound of formula I-C, 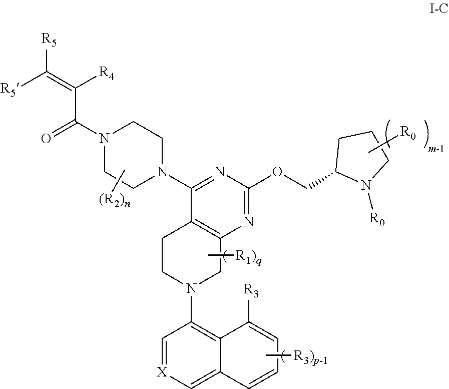
wherein,
X is —CR₆═ or —N═;
on the pyrrolidine ring, R₀ linked to the nitrogen atom is alkyl, methyl-d₃, ethyl-d₅, ethyl-2,2,2-d₃, isopropyl-d₇, methyl-¹⁴C, methyl-¹³C or methyl-¹¹C; the remaining R₀ is hydrogen, deuterium, halogen, alkyl, cycloalkyl, cyano, amino, hydroxy, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₀ is hydrogen, alkyl, cycloalkyl, methyl-d₃, ethyl-d₅, ethyl-2,2,2-d₃, isopropyl-d₇, methyl-¹⁴C, methyl-¹³C or methyl-¹¹C; more preferably, the remaining R₀ is hydrogen, alkyl, methyl-d₃, ethyl-d₅, ethyl-2,2,2-d₃, isopropyl-d₇, methyl-¹⁴C, methyl-¹³C, or methyl-¹¹C;
each R₁ is independently hydrogen, deuterium, halogen, alkyl, cycloalkyl, cyano, amino, haloalkyl or haloalkoxy, preferably hydrogen, deuterium, halogen, alkyl or cycloalkyl, and more preferably hydrogen;
each R₂ is independently hydrogen, deuterium, halogen, alkyl, cycloalkyl, cyano, cyanoalkyl, alkoxyalkyl, alkylaminoalkyl, amino, alkylamino, hydroxy, alkoxy, haloalkyl or haloalkoxy, preferably alkyl, cycloalkyl, cyano or cyanoalkyl, and more preferably alkyl or cyanoalkyl;
on the aromatic ring containing X, R₃, together with the tetrahydropyridopyrimidine ring, in the 1,8-disubstituted form is halogen, alkyl, methyl-d₃, ethyl-d₅, ethyl-2,2,2-d₃, isopropyl-d₇, methyl-¹⁴C, methyl-¹³C or methyl-¹¹C; the remaining R₃ is hydrogen, deuterium, halogen, alkyl, cycloalkyl, cyano, amino, haloalkyl, methyl-d₃, ethyl-d₅, ethyl-2,2,2-d₃, isopropyl-d₇, methyl-¹⁴C, methyl-¹³C or methyl-¹¹C; preferably, the remaining R₃ is hydrogen, halogen, alkyl, methyl-d₃, ethyl-d₅, ethyl-2,2,2-d₃, isopropyl-d₇, methyl-¹⁴C, methyl-¹³C or methyl-¹¹C; more preferably, the remaining R₃ is hydrogen, halogen, alkyl, methyl-d₃, methyl-¹⁴C, methyl-¹³C or methyl-¹¹C;
R₄, R₅ and R_{5′} are each independently hydrogen, deuterium, halogen, alkyl, cycloalkyl, alkylaminoalkyl, cycloalkylaminoalkyl, amino or haloalkyl, preferably hydrogen, deuterium, halogen, alkylaminoalkyl, cycloalkylaminoalkyl or haloalkyl, and more preferably hydrogen, halogen or cycloalkylaminoalkyl;
R₆ is hydrogen, deuterium, halogen, alkyl, cycloalkyl, cyano, amino, haloalkyl or haloalkoxy, preferably hydrogen, deuterium, halogen, cyano or amino, and more preferably hydrogen;
m and p are each independently 1 or 2, and n and q are each independently 0, 1 or 2;
and
if present, at least one R₀ or R₃ is methyl-d₃, ethyl-d₅, ethyl-2,2,2-d₃, isopropyl-d₇, methyl-¹⁴C, methyl-¹³C, or methyl-¹¹C.

7. The compound according to claim 3, 5 or 6, wherein,
X is —CH═ or —N═;
each R₀ is independently alkyl, methyl-d₃, ethyl-d₅, ethyl-2,2,2-d₃, isopropyl-d₇, methyl-¹⁴C, methyl-¹³C, or methyl-¹¹C, wherein the alkyl is C₁-C₆ alkyl, preferably methyl, ethyl, or isopropyl, and more preferably methyl;
R₁ is hydrogen;
each R₂ is independently alkyl or cyanoalkyl, wherein the alkyl is C₁-C₆ alkyl, preferably methyl, ethyl or isopropyl, and more preferably methyl; the cyanoalkyl is —(C₁-C₆ alkylene)-CN, preferably cyanomethyl (—CH₂CN), 1-cyanoethyl (—CH(CN)CH₃) or 2-cyanoethyl (—CH₂CH₂CN), and more preferably cyanomethyl;
each R₃ is independently halogen, alkyl, methyl-d₃, methyl-¹⁴C, methyl-¹³C, or methyl-¹¹C, wherein the halogen is fluorine, chlorine, bromine, or iodine, preferably fluorine, chlorine, or bromine, and more preferably chlorine; the alkyl is C₁-C₆ alkyl, preferably methyl, ethyl or isopropyl, and more preferably methyl;
R₄, R₅ and R_{5′} are each independently hydrogen, halogen or cycloalkylaminoalkyl, wherein the halogen is fluorine, chlorine, bromine or iodine, preferably fluorine, chlorine or bromine, and more preferably fluorine; the cycloalkylaminoalkyl is —(C₁-C₄ alkylene)-NH—(C₃-C₆ cycloalkyl), preferably cyclopropylaminomethyl (c-PrNHCH₂—), cyclobutylaminomethyl (c-BuNHCH₂—), cyclopentylaminomethyl (c-PenNHCH₂—) or cyclohexylaminomethyl (c-HexNHCH₂—), and more preferably cyclopropylaminomethyl;
m, n, p and q are each independently 1 or 2, preferably 1;
and
at least one R₀ or R₃ is methyl-d₃, ethyl-d₅, ethyl-2,2,2-d₃, isopropyl-d₇, methyl-¹⁴C, methyl-¹³C, or methyl-¹¹C.

8. The following compounds or a pharmaceutically acceptable salt, a solvate, a hydrate, a stereoisomer, a tautomer, an isotopically labeled compound or a prodrug thereof or a mixture thereof in any proportion:

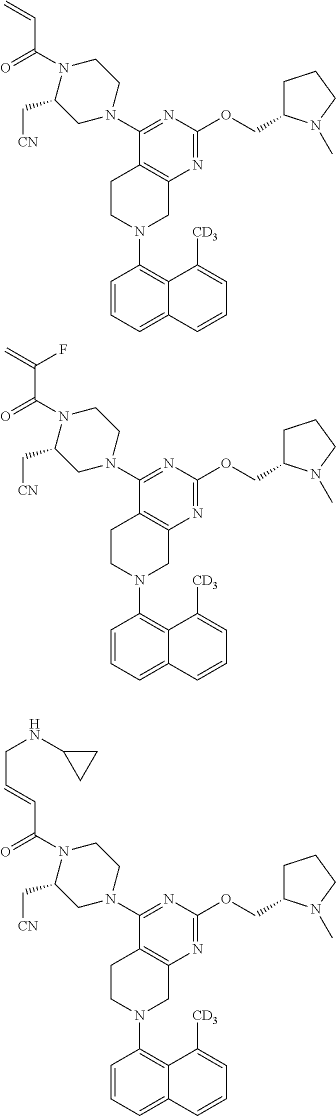 [SMILES: [2H]C([2H])([2H])c1cccc2cccc(N3CCc4c(nc(OC[C@@H]5CCCN5C)nc4N4CCN(C(=O)C=C)[C@@H](CC#N)C4)C3)c12 ; [2H]C([2H])([2H])c1cccc2cccc(N3CCc4c(nc(OC[C@@H]5CCCN5C)nc4N4CCN(C(=O)C(=C)F)[C@@H](CC#N)C4)C3)c12 ; [2H]C([2H])([2H])c1cccc2cccc(N3CCc4c(nc(OC[C@@H]5CCCN5C)nc4N4CCN(C(=O)/C=C/CNC5CC5)[C@@H](CC#N)C4)C3)c12]

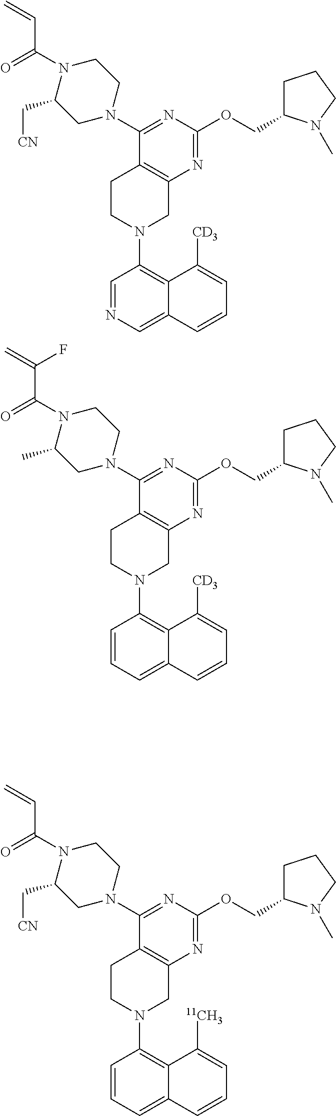 [SMILES: [2H]C([2H])([2H])c1cccc2cncc(N3CCc4c(nc(OC[C@@H]5CCCN5C)nc4N4CCN(C(=O)C=C)[C@@H](CC#N)C4)C3)c12 ; [2H]C([2H])([2H])c1cccc2cccc(N3CCc4c(nc(OC[C@@H]5CCCN5C)nc4N4CCN(C(=O)C(=C)F)[C@@H](C)C4)C3)c12 ; C=CC(=O)N1CCN(c2nc(OC[C@@H]3CCCN3C)nc3c2CCN(c2cccc4cccc([11CH3])c24)C3)C[C@@H]1CC#N]

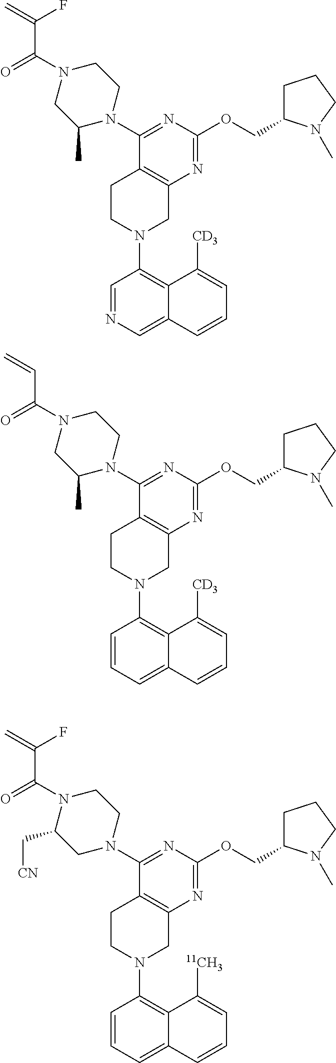 [SMILES: [2H]C([2H])([2H])c1cccc2cncc(N3CCc4c(nc(OC[C@@H]5CCCN5C)nc4N4CCN(C(=O)C(=C)F)C[C@@H]4C)C3)c12 ; [2H]C([2H])([2H])c1cccc2cccc(N3CCc4c(nc(OC[C@@H]5CCCN5C)nc4N4CCN(C(=O)C=C)C[C@@H]4C)C3)c12 ; C=C(F)C(=O)N1CCN(c2nc(OC[C@@H]3CCCN3C)nc3c2CCN(c2cccc4cccc([11CH3])c24)C3)C[C@@H]1CC#N]

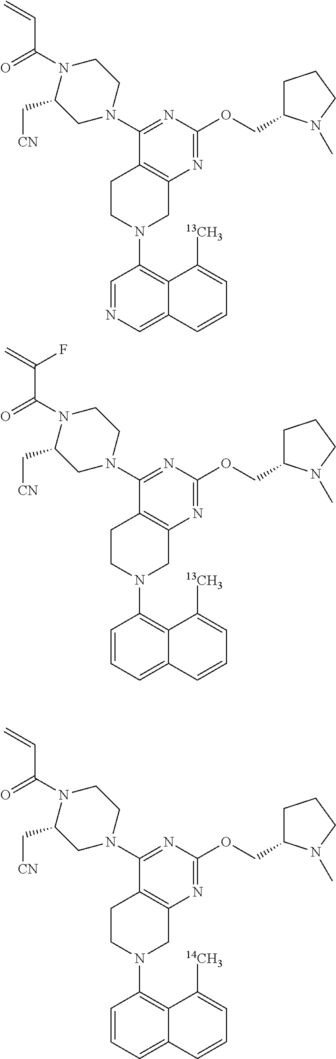 [SMILES: C=CC(=O)N1CCN(c2nc(OC[C@@H]3CCCN3C)nc3c2CCN(c2cncc4cccc([13CH3])c24)C3)C[C@@H]1CC#N ; C=C(F)C(=O)N1CCN(c2nc(OC[C@@H]3CCCN3C)nc3c2CCN(c2cccc4cccc([13CH3])c24)C3)C[C@@H]1CC#N ; C=CC(=O)N1CCN(c2nc(OC[C@@H]3CCCN3C)nc3c2CCN(c2cccc4cccc([14CH3])c24)C3)C[C@@H]1CC#N]

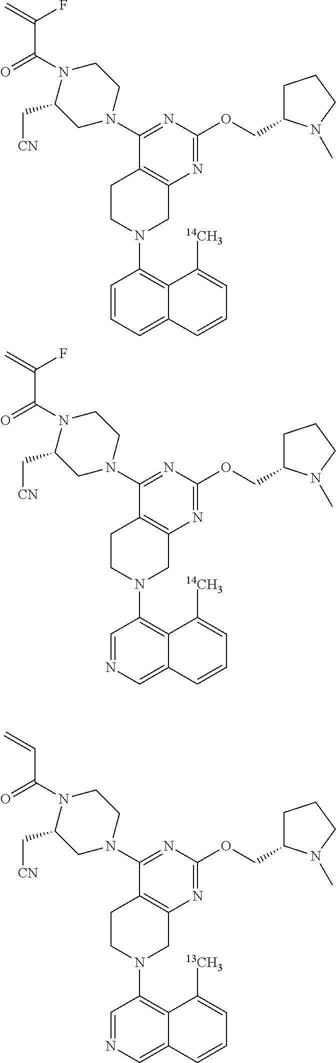 [SMILES: C=C(F)C(=O)N1CCN(c2nc(OC[C@@H]3CCCN3C)nc3c2CCN(c2cccc4cccc([14CH3])c24)C3)C[C@@H]1CC#N ; C=C(F)C(=O)N1CCN(c2nc(OC[C@@H]3CCCN3C)nc3c2CCN(c2cncc4cccc([14CH3])c24)C3)C[C@@H]1CC#N ; C=CC(=O)N1CCN(c2nc(OC[C@@H]3CCCN3C)nc3c2CCN(c2cncc4cccc([13CH3])c24)C3)C[C@@H]1CC#N]

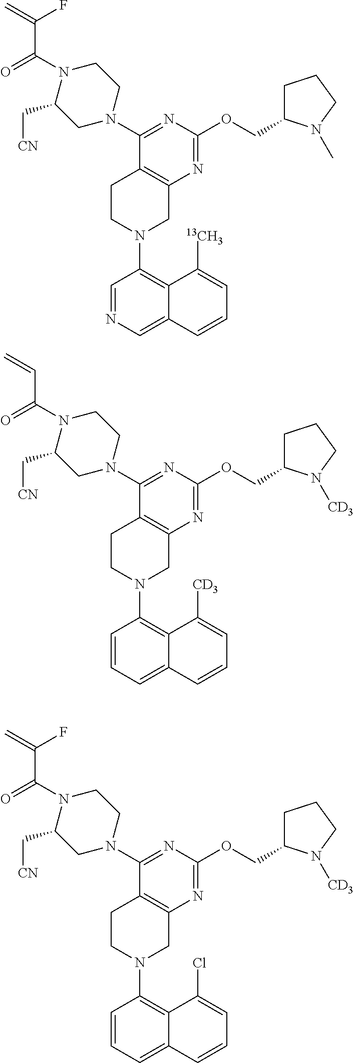 [SMILES: C=C(F)C(=O)N1CCN(c2nc(OC[C@@H]3CCCN3C)nc3c2CCN(c2cncc4cccc([13CH3])c24)C3)C[C@@H]1CC#N ; [2H]C([2H])([2H])c1cccc2cccc(N3CCc4c(nc(OC[C@@H]5CCCN5C([2H])([2H])[2H])nc4N4CCN(C(=O)C=C)[C@@H](CC#N)C4)C3)c12 ; [2H]C([2H])([2H])N1CCC[C@H]1COc1nc2c(c(N3CCN(C(=O)C(=C)F)[C@@H](CC#N)C3)n1)CCN(c1cccc3cccc(Cl)c13)C2]

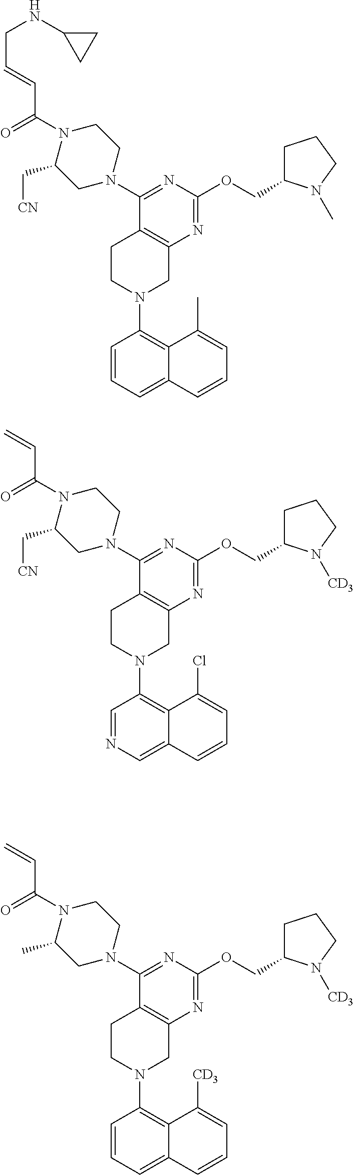 [SMILES: Cc1cccc2cccc(N3CCc4c(nc(OC[C@@H]5CCCN5C)nc4N4CCN(C(=O)/C=C/CNC5CC5)[C@@H](CC#N)C4)C3)c12 ; [2H]C([2H])([2H])N1CCC[C@H]1COc1nc2c(c(N3CCN(C(=O)C=C)[C@@H](CC#N)C3)n1)CCN(c1cncc3cccc(Cl)c13)C2 ; [2H]C([2H])([2H])c1cccc2cccc(N3CCc4c(nc(OC[C@@H]5CCCN5C([2H])([2H])[2H])nc4N4CCN(C(=O)C=C)[C@@H](C)C4)C3)c12]

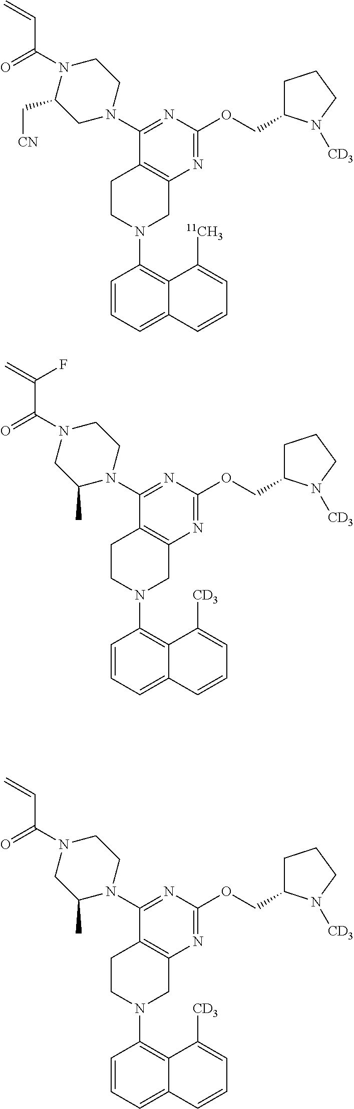 [SMILES: [2H]C([2H])([2H])N1CCC[C@H]1COc1nc2c(c(N3CCN(C(=O)C=C)[C@@H](CC#N)C3)n1)CCN(c1cccc3cccc([11CH3])c13)C2 ; [2H]C([2H])([2H])c1cccc2cccc(N3CCc4c(nc(OC[C@@H]5CCCN5C([2H])([2H])[2H])nc4N4CCN(C(=O)C(=C)F)C[C@@H]4C)C3)c12 ; [2H]C([2H])([2H])c1cccc2cccc(N3CCc4c(nc(OC[C@@H]5CCCN5C([2H])([2H])[2H])nc4N4CCN(C(=O)C=C)C[C@@H]4C)C3)c12]

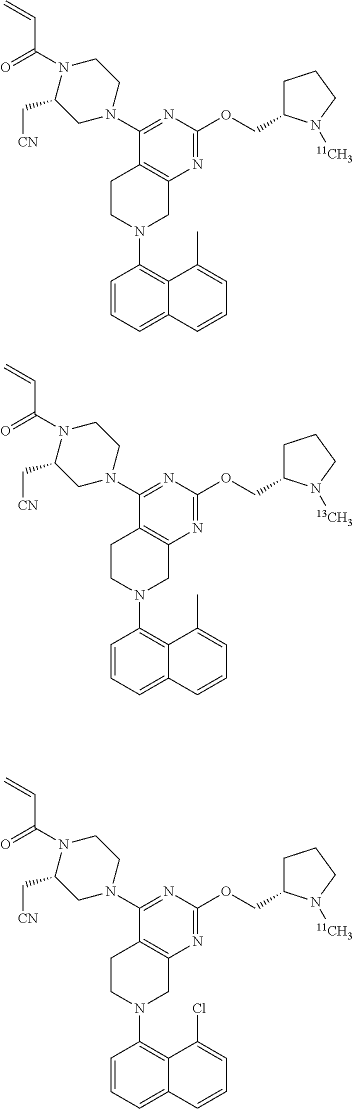 [SMILES: C=CC(=O)N1CCN(c2nc(OC[C@@H]3CCCN3[11CH3])nc3c2CCN(c2cccc4cccc(C)c24)C3)C[C@@H]1CC#N ; C=CC(=O)N1CCN(c2nc(OC[C@@H]3CCCN3[13CH3])nc3c2CCN(c2cccc4cccc(C)c24)C3)C[C@@H]1CC#N ; C=CC(=O)N1CCN(c2nc(OC[C@@H]3CCCN3[11CH3])nc3c2CCN(c2cccc4cccc(Cl)c24)C3)C[C@@H]1CC#N]

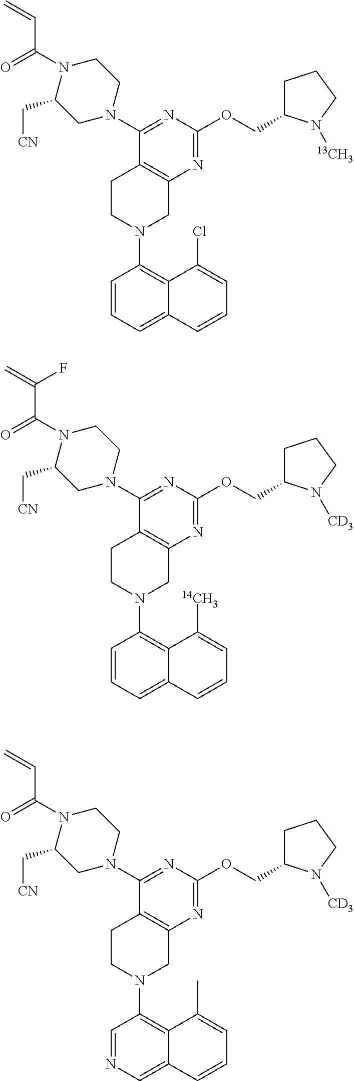 [SMILES: C=CC(=O)N1CCN(c2nc(OC[C@@H]3CCCN3[13CH3])nc3c2CCN(c2cccc4cccc(Cl)c24)C3)C[C@@H]1CC#N ; [2H]C([2H])([2H])N1CCC[C@H]1COc1nc2c(c(N3CCN(C(=O)C(=C)F)[C@@H](CC#N)C3)n1)CCN(c1cccc3cccc([14CH3])c13)C2 ; [2H]C([2H])([2H])N1CCC[C@H]1COc1nc2c(c(N3CCN(C(=O)C=C)[C@@H](CC#N)C3)n1)CCN(c1cncc3cccc(C)c13)C2]

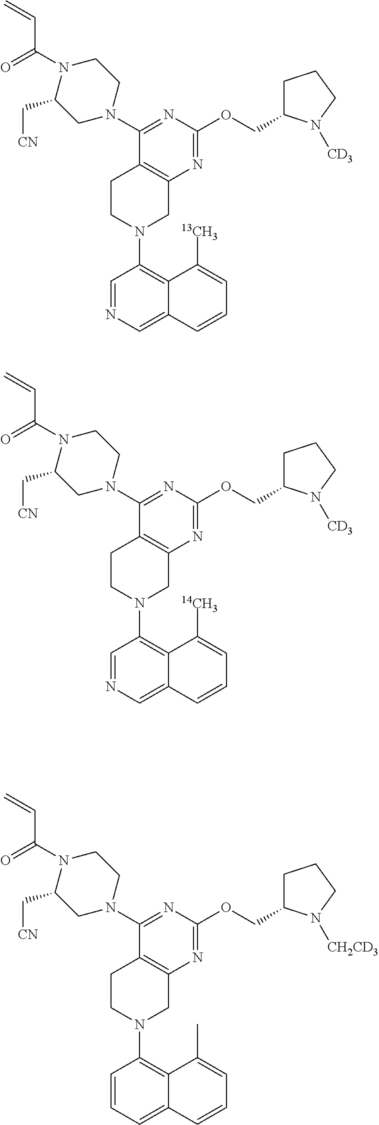

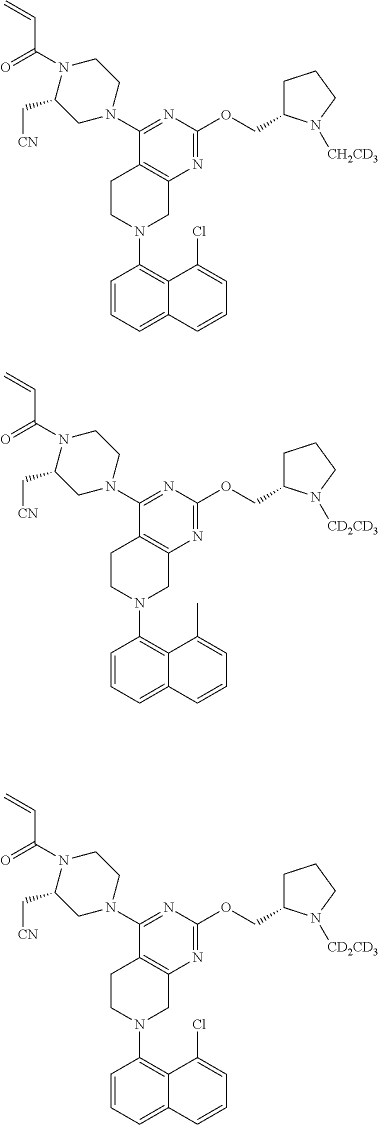

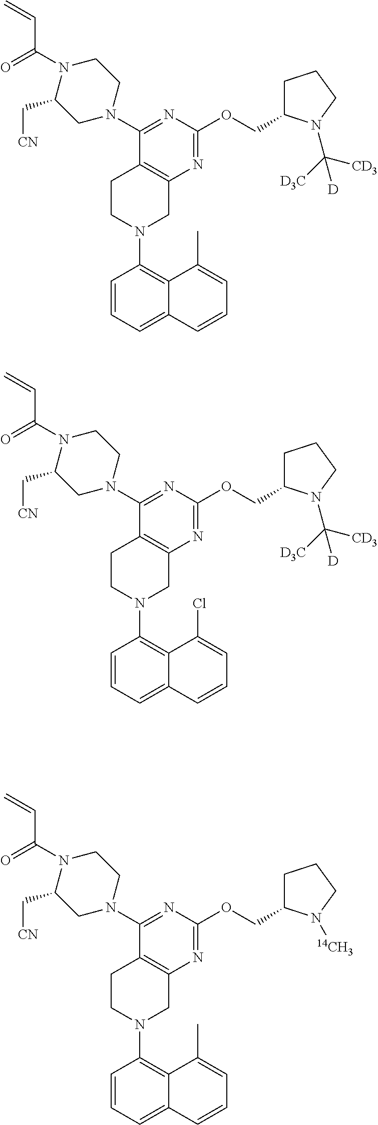 [SMILES: [2H]C([2H])([2H])C([2H])(N1CCC[C@H]1COc1nc2c(c(N3CCN(C(=O)C=C)[C@@H](CC#N)C3)n1)CCN(c1cccc3cccc(C)c13)C2)C([2H])([2H])[2H] ; [2H]C([2H])([2H])C([2H])(N1CCC[C@H]1COc1nc2c(c(N3CCN(C(=O)C=C)[C@@H](CC#N)C3)n1)CCN(c1cccc3cccc(Cl)c13)C2)C([2H])([2H])[2H] ; C=CC(=O)N1CCN(c2nc(OC[C@@H]3CCCN3[14CH3])nc3c2CCN(c2cccc4cccc(C)c24)C3)C[C@@H]1CC#N]

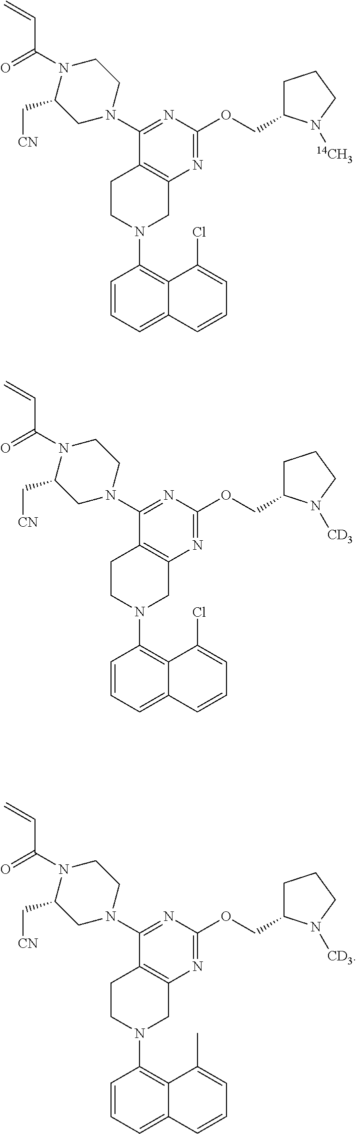 [SMILES: C=CC(=O)N1CCN(c2nc(OC[C@@H]3CCCN3[14CH3])nc3c2CCN(c2cccc4cccc(Cl)c24)C3)C[C@@H]1CC#N ; [2H]C([2H])([2H])N1CCC[C@H]1COc1nc2c(c(N3CCN(C(=O)C=C)[C@@H](CC#N)C3)n1)CCN(c1cccc3cccc(Cl)c13)C2 ; [2H]C([2H])([2H])N1CCC[C@H]1COc1nc2c(c(N3CCN(C(=O)C=C)[C@@H](CC#N)C3)n1)CCN(c1cccc3cccc(C)c13)C2]

9. A method for preparing the compound of formula I according to claim 1, comprising:
1) reacting compound I-1 with compound I-2 to obtain compound I-3; 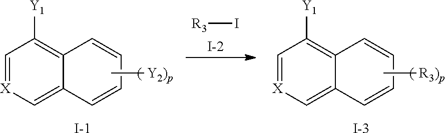
2) reacting compound I-3 with compound I-4 to obtain compound I-5; 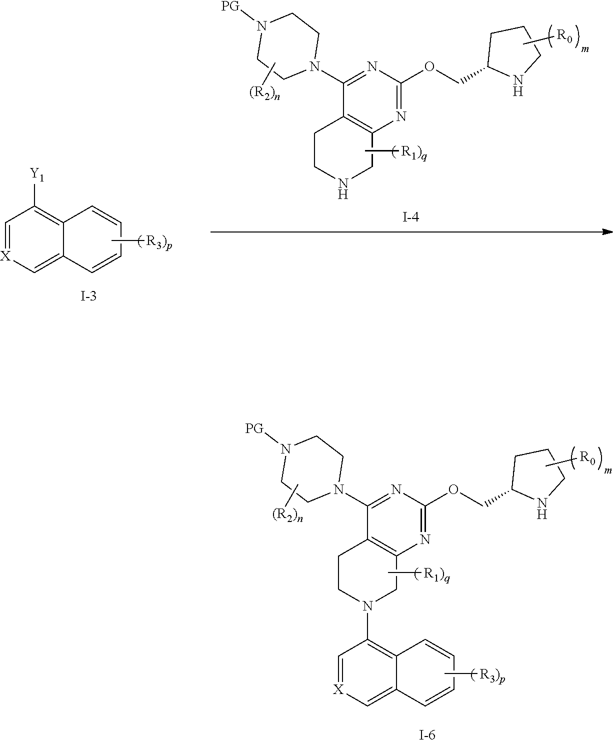
3) subjecting compound I-5 to deprotection reaction to obtain compound I-6; and 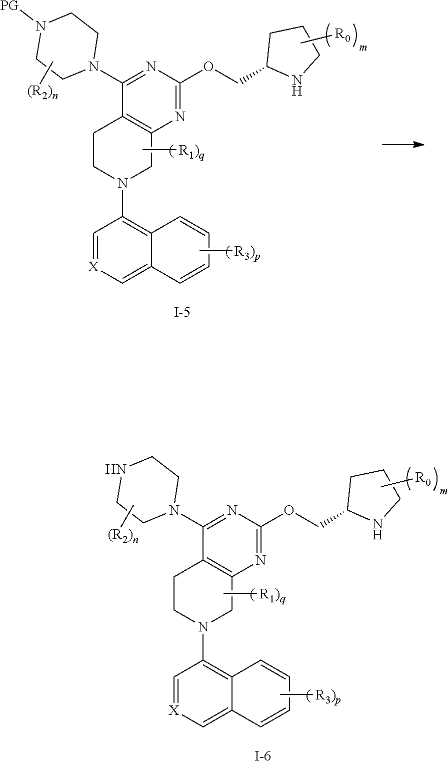
4) reacting compound I-6 with compound I-7 to obtain the compound of formula I; 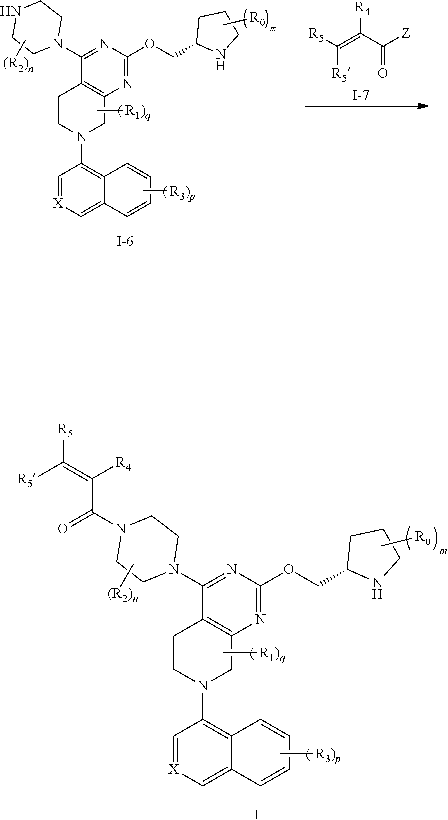
wherein Y₁ and Y₂ are each independently chlorine, bromine, iodine, methanesulfonyloxy, trifluoromethanesulfonyloxy, p-toluenesulfonyloxy, a borate ester, a zinc halide group, a magnesium halide group, or a tin halide group; z is hydroxy, bromine or chlorine; PG represents a protecting group; x, R₀, R₁, R₂, R₃, R₄, R₅, R_{5′}, m, n, p and q are as defined in claim 1.

10. A pharmaceutical composition comprising the compound or the pharmaceutically acceptable salt, the solvate, the hydrate, the stereoisomer, the tautomer, the isotopically labeled compound or the prodrug thereof or the mixture thereof in any proportion according to any one of claims 1 to 9, and a pharmaceutically acceptable carrier.

11. The compound or the pharmaceutically acceptable salt, the solvate, the hydrate, the stereoisomer, the tautomer, the isotopically labeled compound or the prodrug thereof or the mixture thereof in any proportion according to any one of claims 1 to 8, or the pharmaceutical composition according to claim 10, for use as a KRAS G12C protein inhibitor.

12. Use of the compound or the pharmaceutically acceptable salt, the solvate, the hydrate, the stereoisomer, the tautomer, the isotopically labeled compound or the prodrug thereof or the mixture thereof in any proportion according to any one of claims 1 to 8, or the pharmaceutical composition according to claim 10, in preparing a medicament for preventing and/or treating a disease at least partially mediated by KRAS G12C protein.

13. A method for preventing and/or treating a disease at least partially mediated by KRAS G12C protein comprising: administering to an individual in need thereof a therapeutically effective amount of the compound or the pharmaceutically acceptable salt, the solvate, the hydrate, the stereoisomer, the tautomer, the isotopically labeled compound or the prodrug thereof or the mixture thereof in any proportion according to any one of claims 1 to 8, or the pharmaceutical composition according to claim 10.

14. A pharmaceutical combination form comprising the compound or the pharmaceutically acceptable salt, the solvate, the hydrate, the stereoisomer, the tautomer, the isotopically labeled compound or the prodrug thereof or the mixture thereof in any proportion according to any one of claims 1 to 8, or the pharmaceutical composition according to claim 10, and at least one additional therapeutic agent for cancer.

15. A method for preventing and/or treating cancer comprising: administering to an individual in need thereof a therapeutically effective amount of the compound or the pharmaceutically acceptable salt, the solvate, the hydrate, the stereoisomer, the tautomer, the isotopically labeled compound or the prodrug thereof or the mixture thereof in any proportion according to any one of claims 1 to 8, or the pharmaceutical composition according to claim 10, or the pharmaceutical combination form according to claim 14.
